SEQUENCE LISTING (18 sequences)

SEQ ID NO: 1 - DNA, 2814 bp, synthetic construct
  source 1..2814: mol_type = other DNA
atggccaccccctctatgatgccacaatgggcttatatgcatattgccggtcaagacgcgtctgaatacctgtcacccggacttgtgcaattcgcaagagccacggatacctattttagtatgggcaacaagtttcggaaccctacagtagctcccacgcacgacgttactacagatagatcccaacggctgatgctgcggttcgttcctgtggaccgcgaagataacacttatagttataaagtgagatacacccttgctgtaggagacaatcgagtgctggacatggcttcaacctttttcgatatacggggagtattggatcgcggcccttctttcaaaccatattccggcacagcctataatagcctcgcacccaaaggtgcccctaatacaagtcagtggattgtaaccgcaggagaggaacgcgccgtgactacaacaacaaatacatttggaattgcatctatgaaaggggataatataaccaaagagggcctggaaatcggtaaagacattactgccgataacaagcccatttacgcggataagacttatcagccagaacctcaggtgggcgaagaaagctggacagacacagatggaactaacgaaaaattcggaggtagagctttgaaacccgccactaaaatgaaaccatgctacggtagcttcgctcgacctaccaacattaagggaggccaagctaaaaatcggaaagtgaaacctacagaaggcgatgtggagaccgaggaacccgacatagatatggagtttttcgatggtcgggaagccgccgatgccttttctccagagatcgtgctttacacggaaaacgttaatctggaaactccagatagccacgttgtgtacaagccaggaactagcgacgacaactcccacgccaacctcggccagcaggcaatgcccaacagacctaactacattggatttagagataacttcgtggggcttatgtactacaactccaccggcaacatgggtgtccttgcaggacaagctagtcagcttaatgcagtggttgatctccaggatcgcaataccgaattgtcttatcaactgcttttggatagtcttggggaccggacaaggtatttctctatgtggaatcaggctgtggatagttacgaccccgatgtccggatcattgaaaatcatgggattgaagacgagctgcctaattattgctttccccttgacggcatcggcccagctaagacataccagggcattaaaagcaaagacaacggatgggagaaggatgataatgtatcaaaatctaatgagatcgcaatcggcaacaaccaggcgatggaaattaacattcaggcaaatctgtggagatcctttttgtatagcaatgtcgcgctgtatttgccagacgtatacaagtacactccgaccaacataaccctgcccgctaatactaacacttatgaatacatgaacggacgagtggtaagccccagcttggtggacagttacatcaacatcggcgcccgctggagccttgatcctatggataacgtcaatcccttcaatcaccatagaaatgcaggcctgcggtatcggtctatgcttctcggcaatgggagatatgtcccctttcacattcaagtgcctcagaaattcttcgcagtgaaaaacctgcttctcttgccggggtcctatacctatgaatggaatttccggaaggatgtcaacatggttctgcaatctagcttgggaaatgatcttagaacggacggcgctaccatctctttcaccagtattaacctttacgctactttctttccaatggcccacaacactgcatccaccctggaggccatgctcaggaatgataccaatgaccagagctttaatgattacctctctgcagccaatatgttgtatccgatcccggcgaacgccaccaacattcccatctctataccgtctcgcaattgggccgcttttagagggtggagctttacacggctgaagactaaggagaccccctcccttggctcaggatttgacccttactttgtgtactctgggagtatcccctatttggacggcactttttatctgaaccacacattcaagaaagtttctatcatgttcgactcttcagtttcttggccaggaaacgatagattgcttagccccaacgaatttgaaattaaacgcacagtcgacggagaaggatataatgtcgctcagtgcaacatgaccaaagattggtttctggtgcaaatgctcgctaactataacataggctatcagggcttctatattcctgaaggctataaagataggatgtattcattttttaggaacttccaaccaatgtctaggcaagtcgtggacgaggtaaactacacagactataaggccgtgactctcccatatcagcataacaattcaggctttgtgggatacttggcaccaactatgcgccagggtgagccctatcccgccaactacccttaccccttgattggtacaactgcagtgaagtcagtgacccaaaaaaagtttctttgcgatcgcactatgtggagaatccctttctcctccaatttcatgagtatgggagctctcactgatctcggtcagaacatgttgtatgccaatagcgcacatgcgctcgatatgacatttgaagttgatccgatggacgagcctaccctgctctatttgctgtttgaggtttttgacgtcgtgagagtgcaccaacctcatcggggcgtgattgaagctgtatacttgcgcacacccttctcagccgggaacgctaccacttaa

SEQ ID NO: 2 - protein, 937 aa, synthetic construct
MATPSMMPQWAYMHIAGQDASEYLSPGLVQFARATDTYFSMGNKFRNPTVAPTHDVTTDRSQRLMLRFVPVDREDNTYSYKVRYTLAVGDNRVLDMASTFFDIRGVLDRGPSFKPYSGTAYNSLAPKGAPNTSQWIVTAGEERAVTTTTNTFGIASMKGDNITKEGLEIGKDITADNKPIYADKTYQPEPQVGEESWTDTDGTNEKFGGRALKPATKMKPCYGSFARPTNIKGGQAKNRKVKPTEGDVETEEPDIDMEFFDGREAADAFSPEIVLYTENVNLETPDSHVVYKPGTSDDNSHANLGQQAMPNRPNYIGFRDNFVGLMYYNSTGNMGVLAGQASQLNAVVDLQDRNTELSYQLLLDSLGDRTRYFSMWNQAVDSYDPDVRIIENHGIEDELPNYCFPLDGIGPAKTYQGIKSKDNGWEKDDNVSKSNEIAIGNNQAMEINIQANLWRSFLYSNVALYLPDVYKYTPTNITLPANTNTYEYMNGRVVSPSLVDSYINIGARWSLDPMDNVNPFNHHRNAGLRYRSMLLGNGRYVPFHIQVPQKFFAVKNLLLLPGSYTYEWNFRKDVNMVLQSSLGNDLRTDGATISFTSINLYATFFPMAHNTASTLEAMLRNDTNDQSFNDYLSAANMLYPIPANATNIPISIPSRNWAAFRGWSFTRLKTKETPSLGSGFDPYFVYSGSIPYLDGTFYLNHTFKKVSIMFDSSVSWPGNDRLLSPNEFEIKRTVDGEGYNVAQCNMTKDWFLVQMLANYNIGYQGFYIPEGYKDRMYSFFRNFQPMSRQVVDEVNYTDYKAVTLPYQHNNSGFVGYLAPTMRQGEPYPANYPYPLIGTTAVKSVTQKKFLCDRTMWRIPFSSNFMSMGALTDLGQNMLYANSAHALDMTFEVDPMDEPTLLYLLFEVFDVVRVHQPHRGVIEAVYLRTPFSAGNATT

SEQ ID NO: 3 - DNA, 1635 bp, synthetic construct
  source 1..1635: mol_type = other DNA
atgagaagacgcgccgtgcttggcggagcaatggtttatccagaaggacctcccccatcctacgagtcagtaatgcagcagcaagccgccatgatccaaccacctcttgaagctccatttgtacctccgcgctacttggcaccgactgagggcagaaactcaatccgctactcagaacttagcccgctctatgataccacaaaactgtatctcgtagataacaaaagtgctgacatagcttcactcaactaccaaaatgaccattccaattttctcacaacagtggtccaaaacaacgactttactccaacagaagcaagtacacagacaattaattttgacgaaagatcccgctgggggggccagctgaaaactataatgcatacaaatatgcccaacgtaaacgagtacatgttctcaaacaagttcaaggcaagggtgatggtctctcgcaaggcacccgagggcgtaatagtcaacgatacgtatgaccacaaagaagacatcctgaaatacgagtggtttgagttcacactgcccgagggaaattttagtgcaacaatgactattgacttgatgaataatgccattatcgacaactaccttgaaatcggaagacagaatggggtgctcgaatcagacatcggagtcaaattcgacacacggaacttcagactgggctgggaccccgaaacaaaactgatcatgccaggtgtgtacacatatgaagcgtttcatcctgacatagtgttgctccccggatgcggcgtcgacttcacagaatccagattgagcaacctgcttggtatcagaaaacgacatccgtttcaagaaggtttcaaaataatgtatgaggatttggaaggcggtaatatcccagcacttttggacgtaacagcatatgaagagagcaaaaaagacactaccactgagacaacaacactggcagtcgccgaggagacatcagaggatgataacatcacacgcggtgatacctacattacagagaagcaaaagagagaggcagctgccgccgaagtgaaaaaagagctcaagatccaaccattggaaaaggattctaaatctcgaagttacaacgttcttgaagacaaaatcaatacagcctatcgctcctggtatttgtcttataactatgggaacccggaaaaaggtataagaagctggaccctgctgacgacatctgatgtaacatgtggagctgaacaggtttactggagtttgccagatatgatgcaagaccctgtgacattccgaagcacccgacaggtaaacaattaccccgttgtgggcgccgaactcatgcccgtgttctccaagagtttttacaacgaacaggctgtttattcccagcaattgaggcaggccacgtccctgacacacgtcttcaatagatttcccgaaaatcagatcctcatccggcccccagcccctacaataaccacagtaagcgaaaacgtgccagccttgacagaccacggcactctccccctgcgatcttcaatacgcggcgtacagcgagtgacagttacagacgctaggcgaaggacttgcccttatgtttataaggccctcggcatagtggcccctagagtcctgtcctctcggacattctaa

SEQ ID NO: 4 - protein, 544 aa, synthetic construct
MRRRAVLGGAMVYPEGPPPSYESVMQQQAAMIQPPLEAPFVPPRYLAPTEGRNSIRYSELSPLYDTTKLYLVDNKSADIASLNYQNDHSNFLTTVVQNNDFTPTEASTQTINFDERSRWGGQLKTIMHTNMPNVNEYMFSNKFKARVMVSRKAPEGVIVNDTYDHKEDILKYEWFEFTLPEGNFSATMTIDLMNNAIIDNYLEIGRQNGVLESDIGVKFDTRNFRLGWDPETKLIMPGVYTYEAFHPDIVLLPGCGVDFTESRLSNLLGIRKRHPFQEGFKIMYEDLEGGNIPALLDVTAYEESKKDTTTETTTLAVAEETSEDDNITRGDTYITEKQKREAAAAEVKKELKIQPLEKDSKSRSYNVLEDKINTAYRSWYLSYNYGNPEKGIRSWTLLTTSDVTCGAEQVYWSLPDMMQDPVTFRSTRQVNNYPVVGAELMPVFSKSFYNEQAVYSQQLRQATSLTHVFNRFPENQILIRPPAPTITTVSENVPALTDHGTLPLRSSIRGVQRVTVTDARRRTCPYVYKALGIVAPRVLSSRTF

SEQ ID NO: 5 - DNA, 978 bp, synthetic construct
  source 1..978: mol_type = other DNA
atgaccaagcgggtaagactttctgacagtttcaatcctgtctacccctacgaggatgagtctaccagtcagcatcccttcataaaccccggattcatctcccccaatggctttacacaatctcccgatggcgtactgactctcaagtgcttgaccccccttactaccacaggaggtagccttcaactgaaagtcggcggaggcttgactatagacgacacagacggatttctcaaagaaaatattagcgctgcaaccccattggttaaaaccggacatagtattggactttccctgggacctggactgggaacaaacgaaaataaactctgcgcaaagctgggtgagggcttgacatttaattccaacaatatttgcatagatgacaatattaacacactttggaccggagtgaacccaacaactgctaattgtcaaataatggcctcttcagaaagtaacgattgcaagttgattctcacactcgtcaaaacaggcgcccttgtgacagcctttgtttatgtaattggggtctcaaacgattttaacatgctcacaactcacaaaaacatcaatttcaccgccgaactgttcttcgatagcacaggcaacctgttgacctccctcagctcccttaaaactcccttgaatcacaagtctggacagaacatggctactggcgctttgaccaacgcaaaaggttttatgccctcaacaaccgcctatcccttcaatgtgaatagtagagaaaaagaaaactatatttatggcacctgttattacactgcatccgatcataccgccttccctatcgatatttctgtcatgctgaatcagagagccctcaacaatgaaacatcttactgcatacgcgtcacctggtcatggaatacaggcgtggctcccgaagttcagacatccgcaaccacactcgttacatcaccattcacattctattatatcagggaggacgattaa

SEQ ID NO: 6 - protein, 325 aa, synthetic construct
MTKRVRLSDSFNPVYPYEDESTSQHPFINPGFISPNGFTQSPDGVLTLKCLTPLTTTGGSLQLKVGGGLTIDDTDGFLKENISAATPLVKTGHSIGLSLGPGLGTNENKLCAKLGEGLTFNSNNICIDDNINTLWTGVNPTTANCQIMASSESNDCKLILTLVKTGALVTAFVYVIGVSNDFNMLTTHKNINFTAELFFDSTGNLLTSLSSLKTPLNHKSGQNMATGALTNAKGFMPSTTAYPFNVNSREKENYIYGTCYYTASDHTAFPIDISVMLNQRALNNETSYCIRVTWSWNTGVAPEVQTSATTLVTSPFTFYYIREDD

SEQ ID NO: 7 - DNA, 2490 bp, synthetic construct
  source 1..2490: mol_type = other DNA
atggaaacccagcccagcctgccaaccagcctccaggccccttctcacctcgcccctagcagcgacgaggaagagagcctgaccacccctccacctagccctgccaccaccaccagcaccctggacgacgaagaggaagaggtggacgctgctcaggaaatgcaggcccaggacaacgtgaaggccgaagagatcgaggccgacgtggaacaggaccccggctacgtgacccctgccgagcacgaggaagaactgaagcggttcatcgacagagaggacgacgaccggcccgagcaccaggccgatggcgatcaccaggaagccggcatcggcgaccaggtggcagattacctgacaggcctgggcggcgaggacgtgctgctgaagcacctggcccggcagagcaacatcgtgaaggacgctctgctggacctgaccgaggtgcccatcagcgtggaagaactgtcccgggcctacgagctgaacctgttcagccctcacatccctcccaagcggcagcccaacggcacatgcgaggccaaccccagactgaacttctaccccgccttcaccgtgcccgaggtgctggccacctaccacatctttttcaagaaccagaaaatccccgtgtcctgccgggccaaccggaccagagccgatgccctgctgaatctgggccctggagccagactgcccgacattgccagcctggaagaggtgcccaagatcttcgagggcctgggcagcgacgaaaccagagccgccaatgctctgcagcagggcgagaacggcatggacgagcaccacagcgccctggtggaactggaaggcgacaacgcccggctggccgtgctgaagcggagcatcgtggtgacacacttcgcctaccccgccgtgaacctgcctcccaaagtgatgagcgccgtgatggaccatctgctgatcaagagagccggccctctgagcgagaaccagaacatgcaggaccccgacgcctccgacgagggcaagcctgtggtgtccgacgagcagctgagccggtggctgggcaccaacagccccagggacctggaagaacggcggaagctgatgatggccgtggtgctggtgacagtggaactggaatgcctgcggcggttcttcaccgaccccgagacactgcggaagctggaagagaacctgcactacaccttccggcacggcttcgtgcggcaggcttgcaagatcagcaacgtggaactgaccaacctggtgtcctacatgggcatcctgcacgagaaccggctgggccagagcgtgctgcacaccaccctgaagggcgaggccagacgggactacatccgggactgcgtgtacctgtacctgtgccacacctggcagaccggcatgggcgtgtggcagcagtgtctggaagaacagaacctgaaagagctggacaagctgctgcagcggagcctgaaggccctgtggaccggcttcgacgagagaaccgtggccagcgacctggccgacatcatcttccccgagcggctgagagtgaccctgcggaacggcctgcccgacttcatgagccagagcatgctgaacaacttccggtccttcatcctggaaagaagcggcattctgcccgccacctgttgcgccctgcccagcgatttcgtgcccctgacctacagagagtgccctcctcccctgtggtcccactgctacctgttccggctggccaactacctgagctaccacagcgacgtgatcgaggacgtgtccggcgacggcctgctggaatgccactgccggtgcaatctgtgcaccccacacagaagcctggcctgcaaccctcagctgctgagcgagacacagatcatcggcacattcgagctgcaaggccccagctctgagggcgagggaagctctcctggccagagcctgaagctgacccctggcctgtggaccagcgcctacctgcggaagttccaccccgaggactaccacccctacgagatccggttctacgaggaccagtcccaccctcccaaggtggaactgagcgcctgcgtcatcacccagggcgccattctggctcagctgcaggccatccagaagtcccggcaggaatttctgctgaagaaaggcagcggagtgtacctggaccctcagaccggcgaggaactgaacacccggttccctcaggacgtgcccagccccagaaagcaggaagccgagggcgctgccgacgcccctagaggatatggcggcagactgggccagtccggcagaggcggagatggaaggctgggacagcctggcaggggcggaggacagccaggcggcagacagttcggcggaggcagaaggggaggacgcggaggcggcagaagcaacagacggcagaccgtggtgatcggaggcggcgacaaacaggtgccccggcagcagcacggctaccacctgagatctggctctggcggacctgccgccgctcagcagtga

SEQ ID NO: 8 - protein, 829 aa, synthetic construct
METQPSLPTSLQAPSHLAPSSDEEESLTTPPPSPATTTSTLDDEEEEVDAAQEMQAQDNVKAEEIEADVEQDPGYVTPAEHEEELKRFIDREDDDRPEHQADGDHQEAGIGDQVADYLTGLGGEDVLLKHLARQSNIVKDALLDLTEVPISVEELSRAYELNLFSPHIPPKRQPNGTCEANPRLNFYPAFTVPEVLATYHIFFKNQKIPVSCRANRTRADALLNLGPGARLPDIASLEEVPKIFEGLGSDETRAANALQQGENGMDEHHSALVELEGDNARLAVLKRSIVVTHFAYPAVNLPPKVMSAVMDHLLIKRAGPLSENQNMQDPDASDEGKPVVSDEQLSRWLGTNSPRDLEERRKLMMAVVLVTVELECLRRFFTDPETLRKLEENLHYTFRHGFVRQACKISNVELTNLVSYMGILHENRLGQSVLHTTLKGEARRDYIRDCVYLYLCHTWQTGMGVWQQCLEEQNLKELDKLLQRSLKALWTGFDERTVASDLADIIFPERLRVTLRNGLPDFMSQSMLNNFRSFILERSGILPATCCALPSDFVPLTYRECPPPLWSHCYLFRLANYLSYHSDVIEDVSGDGLLECHCRCNLCTPHRSLACNPQLLSETQIIGTFELQGPSSEGEGSSPGQSLKLTPGLWTSAYLRKFHPEDYHPYEIRFYEDQSHPPKVELSACVITQGAILAQLQAIQKSRQEFLLKKGSGVYLDPQTGEELNTRFPQDVPSPRKQEAEGAADAPRGYGGRLGQSGRGGDGRLGQPGRGGGQPGGRQFGGGRRGGRGGGRSNRRQTVVIGGGDKQVPRQQHGYHLRSGSGGPAAAQQ

SEQ ID NO: 9 - DNA, 1158 bp, synthetic construct
  source 1..1158: mol_type = other DNA
atgcatccagtgctgcgacagatgcgcccccagcaacaggccccttctcagcaacagccacaaaaggctcttcttgctcctgcaactactgcagctgcagccgtgagcggcgcgggacagcccgcctatgatctggacttggaagagggcgagggattggcgcgcctgggggctccatcgcccgagcggcacccgcgggtgcaactaaaaaaggactctcgcgaggcatacgtgccccagcagaacctgtttagggacaggagcggcgaggagccagaggagatgcgagcatctcgatttaacgcgggtcgcgagctgcgccacggtctggatcgaagacgggtgctgcaagacgaggattttgaggtcgatgaagtcacagggatcagcccagctagggcacatgtggccgcggccaacctagtctcggcctacgagcagaccgtgaaggaggagcgcaacttccaaaaatcttttaacaaccatgtgcgcaccctgattgcccgcgaggaagtgaccctgggtctgatgcatctgtgggacctgatggaggctatcgcccaaaaccccactagcaaaccactgacagctcagctgtttctggtggttcaacatagcagggacaacgaggcattcagggaggcgttgttgaacatcaccgagcctgatgggagatggctgtatgatctgatcaacatcctgcaaagtattatagtgcaggaacgtagcctgggtttggctgagaaagtggcagctatcaactactcggtcttgagcctgggcaaatactacgctcgcaagatctacaagaccccctacgtacccatagacaaggaggtgaagatagatgggttttacatgcgcatgactctaaaggtactaactctaagcgacgatctgggggtgtatcgcaatgacaggatgcatcgcgcggtgagcgccagtaggaggcgcgagctgagcgacagagaacttatgcacagcttgcaaagagctctaacgggggccgggactgatggggagaactactttgacatgggagcggacttgcaatggcaacccagtcgcagggccatggaggctgcagggtgtgagcttccttacatagaagaggtggatgaagtcgaggacgaggagggcgagtacttggaagactga

SEQ ID NO: 10 - protein, 385 aa, synthetic construct
MHPVLRQMRPQQQAPSQQQPQKALLAPATTAAAAVSGAGQPAYDLDLEEGEGLARLGAPSPERHPRVQLKKDSREAYVPQQNLFRDRSGEEPEEMRASRFNAGRELRHGLDRRRVLQDEDFEVDEVTGISPARAHVAAANLVSAYEQTVKEERNFQKSFNNHVRTLIAREEVTLGLMHLWDLMEAIAQNPTSKPLTAQLFLVVQHSRDNEAFREALLNITEPDGRWLYDLINILQSIIVQERSLGLAEKVAAINYSVLSLGKYYARKIYKTPYVPIDKEVKIDGFYMRMTLKVLTLSDDLGVYRNDRMHRAVSASRRRELSDRELMHSLQRALTGAGTDGENYFDMGADLQWQPSRRAMEAAGCELPYIEEVDEVEDEEGEYLED

SEQ ID NO: 11 - DNA, 1767 bp, synthetic construct
  source 1..1767: mol_type = other DNA
atggaacagcagcaggcccctgaccctgccatgagagccgccctgcagagccagcccagcggcatcaacagcagcgacgactggacccaggccatgcagcggatcatggccctgaccacccggaaccccgaggccttcagacagcagccccaggccaaccggctgagcgccattctggaagccgtggtgcccagccggtccaaccccacccacgagaaggtgctggccatcgtgaacgccctggtggaaaacaaggccatccggcccgatgaggctggcctggtgtacaatgccctgctggaacgggtggcccggtacaactccagcaacgtgcagaccaacctggaccggatggtgacagacgtgcgcgaggccgtgtcccagagagagagattccagcgggacgccaacctgggcagcctggtggccctgaacgccttcctgagcacccagcccgccaacgtgcccagaggccagcaggactacaccaactttctgagcgccctgcggctgatggtggccgaggtgccacagagcgaggtgtaccagagcggccctgactacttcttccagaccagccggcagggactgcagaccgtgaacctgacccaggccttcaagaacctgaagggcctgtggggagtgcacgctcccgtgggagatagagccaccgtgtccagcctgctgacccctaacagcagactcctgctgctgctggtgtccccattcaccgacagcggctccatcgaccggaacagctacctgggctacctgctgaacctgtacagagaggccatcggccagagccaggtggacgagcagacataccaggaaatcacacaggtctccagagccctgggccaggaagataccggcagcctggaagccaccctgaactttctgctgaccaacagaagccagaagatccctccccagtacgccctgaccgccgaggaagaaagaatcctgagatacgtgcagcagagcgtgggcctgttcctgatgcaagaaggcgccacccctacagccgccctggacatgaccgcccggaacatggaaccctctatgtacgccagcaaccggcccttcatcaacaaactgctggactacctgcaccgggctgccgccatgaacagcgactacttcaccaacgccatcctgaaccctcactggctgcctccacccggcttctacaccggcgagtacgacatgcccgaccccaacgacggcttcctgtgggacgacgtggacagcagcatcttcagcccacctcccggctacaccgtgtggaagaaagagggcggcgacacccggcacagcagcgtgtcactgagcagaacagccggcgctgccgctacagtgcctgaggccgccagcccattccccagcctgcccttcagcctgaacagcgtgcggagcagcgagctgggccggatcaccagacccagactgatgggcgaggacgagtacctgaacgacagcctgctgcggcccgagagagagaagaacttccccaacaacggcatcgagagcctggtggacaagatgagccggtggaaaacctacgcccaggaccaccgggacgagcccaagatcctgggcgccaccagccggcccaccagaagaaggcagcggcacgacagacagatgggcctggtctgggatgatgaggactccgccgacgacagctccgtgctggatctgggcggcagaggcggcggaaaccctttcgctcacctgaggcccagattcggccggatgctgtga

SEQ ID NO: 12 - protein, 588 aa, synthetic construct
MEQQQAPDPAMRAALQSQPSGINSSDDWTQAMQRIMALTTRNPEAFRQQPQANRLSAILEAVVPSRSNPTHEKVLAIVNALVENKAIRPDEAGLVYNALLERVARYNSSNVQTNLDRMVTDVREAVSQRERFQRDANLGSLVALNAFLSTQPANVPRGQQDYTNFLSALRLMVAEVPQSEVYQSGPDYFFQTSRQGLQTVNLTQAFKNLKGLWGVHAPVGDRATVSSLLTPNSRLLLLLVSPFTDSGSIDRNSYLGYLLNLYREAIGQSQVDEQTYQEITQVSRALGQEDTGSLEATLNFLLTNRSQKIPPQYALTAEEERILRYVQQSVGLFLMQEGATPTAALDMTARNMEPSMYASNRPFINKLLDYLHRAAAMNSDYFTNAILNPHWLPPPGFYTGEYDMPDPNDGFLWDDVDSSIFSPPPGYTVWKKEGGDTRHSSVSLSRTAGAAATVPEAASPFPSLPFSLNSVRSSELGRITRPRLMGEDEYLNDSLLRPEREKNFPNNGIESLVDKMSRWKTYAQDHRDEPKILGATSRPTRRRQRHDRQMGLVWDDEDSADDSSVLDLGGRGGGNPFAHLRPRFGRML

SEQ ID NO: 13 - DNA, 753 bp, synthetic construct
  source 1..753: mol_type = other DNA
atggaagatattaacttcagctccctggcccctcggcacggcaccagaccctacatgggcacttggagcgacatcggcaccagccagctgaatggcggagccttcaactggtccagcatttggagcggcctgaagaacttcggctccaccgtgaaaacctacggcaacaaggcctggaacagctccaccggccaggccctgcggaacaagctgaaagagcagaacttccagcagaaagtggtggacggaatcgccagcggcatcaacggcgtggtggacctggccaaccaggccgtgcagaagcagatcaactcccggctggaccctcctcccgccacccctggcgagatggaagtggaagaggaactgcctcccctggaaaagcggggcgacaagaggcccagacccgaactggaacagaccctggtgacacgggccgacgagcctcccagctacgaggaagccgtgaagctgggcatgcctaccaccagacccgtggcccacatggccaccggcgtgatgaagcccagccagagccacagacccgccaccctggatctgcctccacctcctgcctctgccgctctggtgcctaagcccgtggccaccagaaagcccaccgccgtgcagcctgtggccgtggctagacctagacctggcggcacccctaggccaaaggccaactggcagagcaccctgaacagcatcgtgggcctgggcgtgcagagcgtgaagcggcggagatgctactga

SEQ ID NO: 14 - protein, 250 aa, synthetic construct
MEDINFSSLAPRHGTRPYMGTWSDIGTSQLNGGAFNWSSIWSGLKNFGSTVKTYGNKAWNSSTGQALRNKLKEQNFQQKVVDGIASGINGVVDLANQAVQKQINSRLDPPPATPGEMEVEEELPPLEKRGDKRPRPELEQTLVTRADEPPSYEEAVKLGMPTTRPVAHMATGVMKPSQSHRPATLDLPPPPASAALVPKPVATRKPTAVQPVAVARPRPGGTPRPKANWQSTLNSIVGLGVQSVKRRRCY

SEQ ID NO: 15 - DNA, 684 bp, synthetic construct
  source 1..684: mol_type = other DNA
atgagcaaagagatccccaccccttacatgtggtcctaccagccccagatgggcctggccgctggcgcctctcaggactacagcacccggatgaactggctgagcgccggacccagcatgatcagcagagtgaacgacatccgggcctaccggaaccagctgctgctggaacagagcgccctgaccaccacccctcggcagcacctgaaccccagaaactggcctgccgccctggtgtaccaggaaacccctgcccctaccaccgtgctgctgcccagagatgcccaggccgaggtgcacatgaccaacgctggcgtgcagctggctggcggctctgccctgtgcagacacagaccccagcagagcatcaagcggctggtgatccggggcagaggcatccagctgaacgacgagagcgtgtccagcagcctgggactgaggcccgacggcgtgttccagatcgccggctgcggcagaagcagcttcacccctagacaggccgtgctgaccctggaaagcagcagcagccagcccagaagcggcggcatcggcaccctgcagttcgtggaagagttcaccccaagcgtgtacttcaaccccttcagcggcagccctggccagtaccccgacgagttcatccccaacttcgacgccatcagcgagagcgtggacggctacgactga

SEQ ID NO: 16 - protein, 227 aa, synthetic construct
MSKEIPTPYMWSYQPQMGLAAGASQDYSTRMNWLSAGPSMISRVNDIRAYRNQLLLEQSALTTTPRQHLNPRNWPAALVYQETPAPTTVLLPRDAQAEVHMTNAGVQLAGGSALCRHRPQQSIKRLVIRGRGIQLNDESVSSSLGLRPDGVFQIAGCGRSSFTPRQAVLTLESSSSQPRSGGIGTLQFVEEFTPSVYFNPFSGSPGQYPDEFIPNFDAISESVDGYD

SEQ ID NO: 17 - DNA, 417 bp, synthetic construct
  source 1..417: mol_type = other DNA
atgagcggctctgccagctttgagggcggcgtgttcagcccctacctgaccggcagactgcctccttgggctggcgtgcggcagaacgtgatgggcagcaccgtggacggcagacccgtgcagcctgccaacagcagcaccctgacctacgctaccctgagcagcagccctctggatgccgctgccgccgctgctgccacagccgccgctaataccatcctgggcatgggctactacggctctatcgtggccaacagttcctccagcaacaaccccagcaccctggccgaggacaagctgctggtgctgctggcccagctggaagccctgacccagagactgggcgagctgagcaaacaggtggcccagctgagagagcagaccgagtctgccgtggccaccgccaagagcaagtga

SEQ ID NO: 18 - protein, 138 aa, synthetic construct
MSGSASFEGGVFSPYLTGRLPPWAGVRQNVMGSTVDGRPVQPANSSTLTYATLSSSPLDAAAAAAATAAANTILGMGYYGSIVANSSSSNNPSTLAEDKLLVLLAQLEALTQRLGELSKQVAQLREQTESAVATAKSK

(19) United States
(12) Patent Application Publication
(10) Pub. No.: US 2025/0197885 A1
(43) Pub. Date: Jun. 19, 2025

(54) RECOMBINANT VIRUS-LIKE PARTICLE CAPSID VACCINES AGAINST ADENOVIRUSES AND COMPOSITIONS, METHODS, AND USE THEREOF

(71) Applicants: TECHNOVAX, INC., Elmsford, NY (US); The Government Of The United States, As Represented By The Secretary Of The Army, Fort Detrick, MD (US)

(72) Inventors: Jose M. GALARZA, Elmsford, NY (US); Ke WEN, Elmsford, NY (US); Robert KUSCHNER, Arlington, VA (US)

(21) Appl. No.: 18/844,674

(22) PCT Filed: Mar. 10, 2023
(86) PCT No.: PCT/US2023/064176

§ 371 Date: Sep. 6, 2024

Related U.S. Application Data
(60) Provisional application No. 63/318,742, filed on Mar. 10, 2022.

(51) Int. Cl. C12N 15/86; A61K 39/00; C07K 14/005

(52) U.S. Cl. CPC C12N 15/86; C07K 14/005; A61K 39/00; C12N 2710/10322; C12N 2710/10323; C12N 2710/10334

(57) ABSTRACT

The present application relates to adenovirus virus-like particles (AdVLPs) and related compositions, plasmids, and methods. The AdVLP can include a recombinant capsid that comprises major capsid adenovirus (AdV) proteins such as a hexon protein, a penton protein, and a fiber protein, and minor capsid/cement AdV proteins, such as a Illa protein, a VI protein, a VIII protein, and a IX protein. The minor capsid/cement AdV proteins structurally support the major capsid AdV proteins. The recombinant capsid of the AdVLP can further include a chaperone AdV protein, and an accessory scaffold AdV protein.

### CROSS REFERENCE TO RELATED APPLICATIONS

This application is a National Stage of International Application No. PCT/US2023/064176, filed Mar. 10, 2023, which claims priority from U.S. Provisional Application No. 63/318,742, filed Mar. 10, 2022. The International Application was published on Sep. 14, 2023 as International Publication No. WO 2023/173114 A2. The entire disclosures of each of the above identified applications are incorporated herein by reference in their entireties.

### STATEMENT OF GOVERNMENT SUPPORT

This invention was made with government support under contract number W81XWH-19-C-01 awarded by the U.S. Army Medical Research and Development Command/Military Infectious Diseases Research Program/Walter Reed Army Institute of Research. The Government has certain rights in the invention.

### SEQUENCE LISTING

The instant application contains a Sequence Listing which has been submitted electronically in XML format and is hereby incorporated by reference in its entirety. Said XML copy, created on May 17, 2023, is named 10050_011788-WOO_SL.xml and is 33.5 bytes in size.

### TECHNICAL FIELD

The present application relates to virus-like particles (VLPs), compositions comprising virus-like particles (VLPs), and methods of producing or delivering such VLPs. More specifically, the present application relates to VLPs of viruses of the Adenovirus family, also identified as adenovirus virus-like particles (AdVLPs).

### BACKGROUND

Adenoviruses (AdVs) are commonly known as versatile vectors, used for gene therapy, oncolytic virotherapy, and vaccine delivery applications^{1,2}. However, AdVs can also cause infection in humans, ranging in severity from mild to severe, and in very rare cases can even be fatal³. AdVs can infect people of all ages, though the majority of cases occur in younger populations in close contact settings, such as daycares, schools, college dormitories, and military barracks⁴⁻⁸. To date, more than 100 types of Human AdVs have been identified, which are classified into 7 species, termed A through G⁹. Tissue tropism of any one specific AdV type is largely linked to its species classification, as most types within a given species have shared tropisms³. AdVs can infect several different tissues, resulting in an array of clinical manifestations including conjunctivitis, myocarditis, gastroenteritis, hepatitis, and, most commonly, respiratory tract illnesses³.

The adenovirus family is comprised of a large group of icosahedral non-enveloped double stranded linear DNA containing viruses that are able to infect a wide range of mammalian, avian and reptilian species. These viruses are classified within the adenoviridae family, which includes the following five genera: Mastadenovirus, Aviadenovirus, Atadenoviurs, Siadenovirus and Ichtadenovirus. Each of these genera includes adenoviruses that infect different species and within each species there are distinct types. For example, the genus Mastadenovirus infects only mammals and includes viruses affecting humans, bovine, ovine, canine species as well as rodents (mice).

All human adenoviruses are within the genus Mastadenovirus and are further classified into seven groups or species, A through G based on genome sequence, agglutination properties, immune cross-reactivity and genome organization. More than 70 types (a term proposed to superseded serotype) have been recognized amongst these groups; e.g., human adenovirus -A (HAdV-A) includes type: 12, 18, 31; HAdV-B includes type: 3, 7, 11, 14, 16, 21, 34, 35, 50, 55; HAdV-C include type: 1, 2, 5, 6; HAdV-D include type: 8-10, 13, 15, 17, 19, 20, 22-30, 32, 33, 36-39, 42-49, 51, 53, 54; HAdV-E includes type: 4; HAdV-F includes type: 40, 41, and HAdV-G includes type: 52. Other genera such as the Aviadenovirus genus include viruses infecting avian species and causing conditions of different levels of significance. In humans, adenovirus primarily cause respiratory infections; however, depending on the infecting serotype, they may also cause various other conditions such as gastroenteritis, conjunctivitis, cystitis, etc.

Adenoviruses are often recognized as the cause of acute respiratory disease (ARD) in military trainees and children. The clinical manifestation of adenovirus respiratory infections may range from common cold symptoms, pharyngitis, bronchitis and pneumonitis to severe illness and death.

New recombinant adenoviruses may arise from the simultaneous infection of humans with two or more types, an event which commonly occurs in military training centers or nursing homes. These events may result in the formation of new recombinant viruses possessing new biological and immunological characteristics that may overcome pre-existing immunity and rapidly disseminate within the human population. Prophylactic interventions for the control of adenovirus infections are limited. For example, human adenovirus (HAdV) species E type 4 (AdV-4) and species B type 7 (AdV-7) are the serotypes responsible for most outbreaks of acute respiratory disease (ARD) among military recruits of whom almost 80% are infected and 20% require hospitalization according to studies evaluating these trainees.

Of particular interest are AdV-4 and AdV-7, which are the most commonly circulating types in the United States¹⁰. Both AdV-4 and AdV-7 are known to cause acute respiratory disease (ARD), and in rare instances can even cause pneumonia^{10,11}. Infections with AdV-7 are typically associated with more severe outcomes than AdV-4¹²⁻¹⁵. These infections can be debilitating for several days, and in some cases require hospitalization^{16,17}. Treatment options are limited to supportive care, as there are no approved antivirals for treating infections with AdVs^{12,18}. Historically, AdVs have been shown to infect up to 80% of military recruits in the United States, 20% of whom required hospitalization¹⁹.

The high incidence of ARD as a result of AdV infection in the U.S. military prompted the development of live-virus oral vaccines against AdV-4 and AdV-7, administered simultaneously. These vaccines were approved by the FDA in the 1970s for use in military personnel and proved largely successful^{3,20,21}, though they have not been made available to the general public. Protection against infection is provided within one week of immunization, and has been shown to last for at least six years²². Adverse effects associated with vaccination are minimal^{20,23,24}. Following the introduction of these vaccines, incidence of AdV infection in military recruits decreased dramatically²⁴. In tandem, rates of hospitalizations resulting from AdV-induced ARD were reduced by more than 90%²¹.

Although these vaccines against AdV-4 and AdV-7 are generally well-tolerated and effective, the use of live AdV-4 and AdV-7 as immunogens has major downsides. Vaccination with live AdV-4 and AdV-7 by oral administration causes an asymptomatic infection of the gastrointestinal tract. Shedding of vaccine-associated AdVs can be detected in stool samples, persisting for up to 28 days after vaccination^{23,25}. If proper hygiene is not practiced, shed virus can be transmitted to people in close contact with vaccine recipients. Additionally, AdVs have the potential to undergo recombination when multiple types infect the same cell, which can generate novel subtypes^{26,27}. Despite the risks associated with the live virus vaccines, continued vaccination is necessary. Between 1999 and 2004, vaccination of military recruits was paused due to supply issues²⁴. During this period, incidence of AdV-4 and AdV-7 infections and consequent ARD promptly returned to pre-vaccine levels²⁸. Thus, alternative vaccine platforms that can provide protection against AdV infection without the risk of vaccine-associated viral shedding need to be explored.

Furthermore, the emergence of new adenovirus serotypes, e.g., HAdV3, HAdV14, HAdV21 and HAdV55 among others, which are potentially able to cause epidemics and deaths, underscores the importance of these pathogens in public and military health.

Therefore, there is a need for the creation, development and production of new vaccines to protect against illnesses caused by adenoviruses in the military and in the general public.

### SUMMARY

In a first aspect, an adenovirus virus-like particle (AdVLP) is provided. The AdVLP comprises a recombinant capsid comprising: a) major capsid adenovirus (AdV) proteins including a hexon protein, a penton protein, and a fiber protein; b) minor capsid/cement AdV proteins, including a IIIa protein, a VI protein, a VIII protein, and a IX protein, wherein the minor capsid/cement AdV proteins structurally support the major capsid AdV proteins; c) a chaperone AdV protein L4-100k; and d) an accessory scaffold AdV protein L1-52/55k.

In another aspect, the AdVLP is an adenovirus type 4 virus-like particle (AdVLP4), adenovirus type 7 virus-like particle (AdVLP7), adenovirus type 14 virus-like particle (AdVLP14), or adenovirus type 55 virus-like particle (AdVLP55).

In another aspect, the major capsid AdV hexon protein comprises SEQ ID NO: 1 or SEQ ID NO: 2, the major capsid AdV penton protein comprises SEQ ID NO: 3 or SEQ ID NO: 4, and the major capsid AdV fiber protein comprises SEQ ID NO: 5 or SEQ ID NO: 6.

In another aspect, the minor capsid/cement Adv protein IIIA protein comprises SEQ ID NO: 11 or SEQ ID NO: 12, the minor capsid/cement Adv protein VI protein comprises protein comprises SEQ ID NO: 13 or SEQ ID NO: 14, the minor capsid/cement Adv protein VIII protein comprises protein comprises SEQ ID NO: 15 or SEQ ID NO: 16, and the minor capsid/cement Adv protein IX protein comprises protein comprises SEQ ID NO: 17 or SEQ ID NO: 18.

In another aspect the chaperone AdV protein L4-100k comprises SEQ ID NO: 7 or SEQ ID NO: 8.

In another aspect, the accessory scaffold AdV L1-52/55k protein comprises SEQ ID NO: 9 or SEQ ID NO: 10.

In another aspect, the AdVLP further comprises an adjuvant. In a further aspect, the adjuvant is aluminum hydroxide or a squalene-based oil-in-water nano-emulsion.

In another aspect, the AdVLP comprises hexon proteins from two different serotypes such that the AdVLP is chimeric.

In another aspect, the hexon protein comprises one or more antigens of a different infectious agent.

In a further aspect, the different infectious agent is SARS-CoV-2.

In a second aspect, an expression plasmid comprising genes encoding adenovirus proteins is provided. The expression plasmid is suitable for the assembly of adenovirus virus-like particles (AdVLPs). The expression plasmid comprises codon-optimized genes encoding: a) major capsid adenovirus (AdV) proteins including a hexon protein, a penton protein, and a fiber protein; b) minor capsid/cement AdV proteins, including a IIIa protein, a VI protein, a VIII protein, and a IX protein; c) a chaperone AdV protein L4-100k; and d) an accessory scaffold AdV protein L1-52/55k.

In another aspect of the expression plasmid, the AdVLP is an adenovirus type 4 virus-like particle (AdVLP4), adenovirus type 7 virus-like particle (AdVLP7), adenovirus type 14 virus-like particle (AdVLP14), or adenovirus type 55 virus-like particle (AdVLP55).

In another aspect of the expression plasmid, the major capsid AdV hexon protein comprises SEQ ID NO: 1 or SEQ ID NO: 2, the major capsid AdV penton protein comprises SEQ ID NO: 3 or SEQ ID NO: 4, and the major capsid AdV fiber protein comprises SEQ ID NO: 5 or SEQ ID NO: 6. The minor capsid/cement Adv protein IIIA protein comprises SEQ ID NO: 11 or SEQ ID NO: 12, the minor capsid/cement Adv protein VI protein comprises protein comprises SEQ ID NO: 13 or SEQ ID NO: 14, the minor capsid/cement Adv protein VIII protein comprises protein comprises SEQ ID NO: 15 or SEQ ID NO: 16, and the minor capsid/cement Adv protein IX protein comprises protein comprises SEQ ID NO: 17 or SEQ ID NO: 18. The chaperone AdV protein L4-100k comprises SEQ ID NO: 7 or SEQ ID NO: 8, the accessory scaffold AdV protein L1-52/55k comprises SEQ ID NO: 9 or SEQ ID NO: 10.

In a third aspect, an immunogenic composition is provided that comprises at least one AdVLP of the present application.

In another aspect of the immunogenic composition, the at least one AdVLP is adenovirus type 4 virus-like particle (AdVLP4), adenovirus type 7 virus-like particle (AdVLP7), adeonovirus type 14 virus-like particle (AdVLP14), or adenovirus type 55 virus-like particle (AdVLP55).

In another aspect of the immunogenic composition, the recombinant capsid of the at least one AdVLP comprises: a) the major capsid AdV hexon protein comprises SEQ ID NO: 1 or SEQ ID NO: 2, the major capsid AdV penton protein comprises SEQ ID NO: 3 or SEQ ID NO: 4, and the major capsid AdV fiber protein comprises SEQ ID NO: 5 or SEQ ID NO: 6; b) the minor capsid/cement Adv protein IIIA protein comprises SEQ ID NO: 11 or SEQ ID NO: 12, the minor capsid/cement Adv protein VI protein comprises protein comprises SEQ ID NO: 13 or SEQ ID NO: 14, the minor capsid/cement Adv protein VIII protein comprises protein comprises SEQ ID NO: 15 or SEQ ID NO: 16, and the minor capsid/cement Adv protein IX protein comprises protein comprises SEQ ID NO: 17 or SEQ ID NO: 18; c) the chaperone AdV protein L4-100k comprises SEQ ID NO: 7 or SEQ ID NO: 8; and d) the accessory scaffold AdV protein L1-52/55k comprises SEQ ID NO: 9 or SEQ ID NO: 10.

In another aspect of the immunogenic composition, the composition is a trivalent composition comprising AdVLP4, AdVLP7, and AdVLP14.

In another aspect of the immunogenic composition, the composition is a quadrivalent composition comprising AdVLP4, AdVLP7, AdVLP14, and AdVLP55.

In another aspect of the immunogenic composition, the composition is suitable for oral, nasal, mucosal, or parenteral administration.

In a fourth aspect, a method of generating an immune response to one or more adenoviruses in a subject is provided. In the method, an effective amount of the immunogenic composition of the present application is administered to the subject.

In another aspect of the method, the one or more adenoviruses are selected from the group consisting of HAdV-E type 4, HAdV-B type 7, HAdV-B type 14, and HAdV-B type 55.

In another aspect of the method, the immune response vaccinates the subject against the one or more adenoviruses. In a further aspect, the subject is a human.

In a fifth aspect, a method of producing an adenovirus virus-like particle (AdVLP) is provided. In the method, at least one expression plasmid is introduced into a host cell under conditions such that the host cell produces the AdVLP, wherein the at least one expression plasmid comprises codon-optimized genes encoding: a) major capsid adenovirus (AdV) proteins including a hexon protein, a penton protein, and a fiber protein; b) minor capsid/cement AdV proteins, including a IIIa protein, a VI protein, a VIII protein, and a IX protein; c) a chaperone AdV protein L4-100k; and d) an accessory scaffold AdV protein L1-52/55k.

In another aspect of the method, a) the major capsid AdV hexon protein comprises SEQ ID NO: 1 or SEQ ID NO: 2, the major capsid AdV penton protein comprises SEQ ID NO: 3 or SEQ ID NO: 4, and the major capsid AdV fiber protein comprises SEQ ID NO: 5 or SEQ ID NO: 6; b) the minor capsid/cement Adv protein IIIA protein comprises SEQ ID NO: 11 or SEQ ID NO: 12, the minor capsid/cement Adv protein VI protein comprises protein comprises SEQ ID NO: 13 or SEQ ID NO: 14, the minor capsid/cement Adv protein VIII protein comprises protein comprises SEQ ID NO: 15 or SEQ ID NO: 16, and the minor capsid/cement Adv protein IX protein comprises protein comprises SEQ ID NO: 17 or SEQ ID NO: 18; c) the chaperone AdV protein L4-100k comprises SEQ ID NO: 7 or SEQ ID NO: 8; and d) the accessory scaffold AdV protein L1-52/55k comprises SEQ ID NO: 9 or SEQ ID NO: 10.

In another aspect of the method, in the host cell is a eukaryotic cell. In a further aspect, the eukaryotic cell is a mammalian cell.

In another aspect of the method, the method further comprises the step of purifying the AdVLP. In a further aspect, the AdVLP is purified from cell lysates and culture supernatants.

### BRIEF DESCRIPTION OF THE DRAWINGS

FIGS. 1A-1B. Schematic of the plasmids A (FIG. 1A) and B (FIG. 1B) used for the production of the AdVLP vaccine. Plasmid A express the major capsid proteins together with the Hexon-100k protein (100K). Plasmid B expresses the minor capsid protein or cement proteins and the scaffold protein 52/55. In FIGS. 1A-1B, all the genes are derived from human adenovirus 55 (HAdV55).

FIG. 2. Adenovirus Virus-Like Particles (AdVLP-55) were produced in suspension cultures of HEK 293 cells and specific proteins from the different purification steps were evaluated via Western blot using an anti-AdV5 polyclonal antibody. Cells producing the AdVLP were lysed by freeze/thaw or sonication followed by clarification, a CsCl step gradient and a final isopycnic CsCl gradient. The culture supernatant (Sup) was collected, clarified, concentrated (28K) and then purified as was done for the cell lysate. Samples from some of the purification stages were analyzed by Western blot. The major capsid protein (hexon, penton and fiber) were detected, and sample 9 illustrates the three major capsid proteins. Similarly, sample 5 (low band from Sup) reveals a similar pattern.

FIGS. 3A-3B. Analysis of fractions by PAGE and Coomassie blue staining of AdVLP55 purified via chromatography (FIGS. 3A-3B). AdVLP55 particles were produced in suspension cultures of mammalian HEK 293 cells and subsequently purified from cell lysates through successive steps including depth filtration, buffer exchange/concentration, filtration and anion exchange chromatography. Column absorbed material was eluted using a salt gradient and the distinct fractions analyzed by Coomassie blue staining of SDS-PAGE gels. Fractions 17 to 28 contain the major protein components of the AdVLP capsid (hexon, penton and fiber).

FIGS. 4A-4B. Electron microscopy images of AdVLP7. Purified AdVLP7 were negatively stained and examined with a Joel electron microscope. FIG. 4A. Negatively stained with phosphotungstic, bar 500 nm; FIG. 4B. Negatively stained with uranyl acetate, bar 200 nm. AdVLP-7 resembles native adenovirus virions in size, morphology and surface appearance.

FIG. 5. Cryo-electron microscopy images of AdVLP7. Samples of purified adenovirus-like particles 7 (AdVLP7) were plunge-frozen in liquid ethane brought to liquid N2 temperature and then examined by cryo-electron microscopy. AdVLPs show structures identical to native adenovirus.

FIG. 6. Negative staining electron microscopy of Adenovirus 55 virus-like particles (AdVLP55). Particles were produced in suspension of HEK 293 cells and purified by two rounds of CsCl gradient centrifugation, one step gradient and one isopycnic gradient. Purified AdVLP55 were negative stained with phosphotungstic acid and examined by electron microscopy. Images illustrate that AdVLP55 particles appear to resemble the structure of native adenovirus in size (˜96.5 nm in diameter), morphology, and overall architecture.

FIG. 7. Antibody response elicited in mice following immunization with a monovalent AdVLP4 vaccine formulated alone and with adjuvant (Alum) or an inactivated adenovirus 4 virus (AdV4) control. The IgG response was assessed by ELISA using purified AdV4 as coating antigen. The AdVLP-4 vaccine formulated with Alum elicited slightly higher IgG ELISA titers than the AdV-4 virus control; whereas the response to the AdVLP-4 alone was slightly lower than that seen with the virus control.

FIG. 8. The antibody response elicited in mice following immunization with an AdVLP combination vaccine (AdVLP4, AdVLP7, and AdVLP14) or an inactivated adenovirus combination (AdV4, AdV7, and AdV14) virus control were assessed by total IgG ELISA using purified AdV4 as coating antigen. The AdVLP Combo vaccine elicited a high IgG immune response, which was slightly higher than the response elicited by the AdV Combo control. Both responses were significantly higher than the pre-immune sera control. The ELISA result of the AdVLP Combo plus adjuvant is not included.

FIG. 9. Neutralization assay was carried out using a recombinant reporter AdV-4 expressing luciferase and mouse serum from animals immunized with a monovalent adenovirus-like particle type 4 vaccine alone (AdVLP4) or admixed with Alum as adjuvant (AdVLP4+Alum). Serum samples from mice immunized with inactivated adenovirus 4 (AdV4) were used as control. Each serum sample was assayed in triplicate and neutralizing activity is expressed as inhibition dose 50 (ID50), which represents the reciprocal of the serum dilution that reduces luciferase activity [relative luminescent units (RLU)] by 50% as compared to the RLU of the virus infected cell control after subtraction of the RLU background of normal cells. The neutralizing activity was considered positive if the ID50 was greater than 1:8.

FIG. 10. Neutralization assay was performed using a recombinant AdV-4 expressing luciferase and mouse serum from animals immunized with a combination of adenovirus-like particle type 4+7+14 vaccine alone (AdVLP4+7+14) or admixed with Alum (AdVLP4+7+14+Alum). Serum samples from mice immunized with a combination of inactivated adenovirus 4, 7, and 14 (AdV4+7+14) were used as control. Each serum sample was assayed in triplicate and neutralizing activity is expressed as inhibition dose 50 (ID50), which represents the reciprocal of the serum dilution that reduces luciferase activity (relative luminescent units (RLU)) by 50% as compared to the RLU of virus infected cell control after subtraction of RLU background in normal cells. The neutralizing activity was considered positive if the ID50 was greater than 1:8. *There was a statically significant difference between the neutralizing activity of the AdVLP4+7+14+Alum and the adenovirus 4+7+14 vaccine control.

FIGS. 11A-11H. Monovalent and Quadrivalent Adenovirus VLPs induce high anti-adenovirus IgG titers. Serum was collected from male (▾) and female (●) mice immunized with the following: adjuvanted monovalent AdVLP4, AdVLP7, AdVLP14, and AdVLP55, quadrivalent AdVLP Quad (a combination of the 4 monovalent VLPs), or virions AdV4, AdV7, AdV14, and AdV55, quadrivalent AdV Quad (a combination of the 4 adenoviruses). Serum total adenovirus-specific titers (total IgG) were compared via ELISA 2-weeks after the final boost (FIGS. 11A, 11C, 11E, 11G). The 50% of the Maximal Binding Dilution (MBD) was determined for each of the sera against their respective adenoviruses (FIGS. 11B, 11D, 11F, 11H). All values are represented as mean±SD (n≥4 per group). Results were analyzed by one-way ANOVA, with differences considered significant at a p-value of 50.05 (*, P≤50.05; **, P≤0.01; ***, P≤0.001). Asterisks indicate comparisons with monovalent AdVLP; tildes indicate comparisons with the quadrivalent AdVLP.

FIGS. 12A-12E. Morphological analysis of AdVLP-7. FIGS. 12A-12B. Electron micrographs of purified AdVLP-7 particles negatively stained with phosphotungstic acid. Scale bars are 500 nm and 200 nm in FIGS. 12A and 12B, respectively. FIGS. 12C-12E. Distribution of particle diameter of (FIG. 12C) AdVLP-7, (FIG. 12D) wild type AdV-7 particles (WT AdV-7), and (FIG. 12E) wild type AdV-7 particles that lack genomic material (empty capsids), as measured by dynamic light scattering.

FIGS. 13A-13G. Protein composition analysis of AdVLP-7. SDS-PAGE analysis of purified AdVLP-7 (VLP), wild type AdV-7 virions (V), empty capsids (EC), and mock transfected cell lysate (−). FIG. 13A. Coomassie blue stained gel used to determine purity of the samples. Western blot analyses were generated using an (FIG. 13B) anti-AdV-5, (FIG. 13C) anti-IIIa, (FIG. 13D) anti-VIII, (FIG. 13E) anti-IX, (FIG. 13F) anti-L1-52/55k antibody, and anti-AdV-14 (FIG. 13G). The sizes of the molecular weight markers are labeled to the left of each blot. Arrows indicate the proteins of interest (hexon (H), penton (P), and fiber (F)). In FIG. 13C, both precursor (pIIIa) and processed IIIa are shown. In FIG. 13D, precursor (pVIII, arrows) and N- and C-terminal processed cleavage products (arrowheads) of VIII are shown. In FIG. 13G, the arrow indicates protein VI (VI).

FIGS. 14A-14D. Assessment of immunogenicity of AdVLP-7 by ELISA. Sera were assessed by ELISA for AdV-7-specific antibodies using plates coated with AdV-7 virions. (FIG. 14A) Total IgG, (FIG. 14B) IgG1, and (FIG. 14C) IgG2a titers were quantified for all animals (n=10/group) and presented as binding titers (dilution at which sera showed half of their maximal binding activity). Binding titers for individual animals are shown as either circles (•) for females or triangles (▾) for males. Dotted line indicates lower limit of detection (starting serum dilution). FIG. 14D. Ratio of IgG1 titers/IgG2a titers. Ratios are only shown for animals that had detectable levels of both IgG1 and IgG2a (n≥8/group). Data are mean+SD in FIGS. 14A-14C, and geometric mean+geometric SD in FIG. 14D. All results were analyzed by one-way ANOVA with Tukey's multiple comparisons test, with p<0.05 considered significant. * p<0.05, ** p<0.01, *** p<0.001, **** p<0.0001. Ampersands (&) indicate comparison with Sham, pound signs (#) indicate comparisons with WT AdV-7, tildes (˜) indicate comparisons with AdVLP-7 alone, and plus signs (+) indicate comparisons with AdVLP-7+Alum.

FIGS. 15A-15I. Assessment of antigen-specific humoral response by ELISA. Sera were assessed by ELISA for antibodies that bind specifically to major capsid proteins (FIG. 15A) hexon, (FIG. 15B) penton, and (FIG. 15C) fiber. Antigen-specific titers were quantified for all animals (n=10/group) and presented as binding titers (dilution at which sera showed half of their maximal binding activity). Binding titers for individual animals are shown as either circles (•) for females or triangles (▾) for males. Dotted line indicates lower limit of detection (starting serum dilution). Data are mean+SD in FIGS. 15A-15C. Results were analyzed by one-way ANOVA with Tukey's multiple comparisons test, with p<0.05 considered significant. * p<0.05, ** p<0.01, *** p<0.001, **** p<0.0001. Ampersands (&) indicate comparison with Sham, pound signs (#) indicate comparisons with WT AdV-7, tildes (˜) indicate comparisons with AdVLP-7 alone, and plus signs (+) indicate comparisons with AdVLP-7+Alum. FIGS. 15D-15F. Correlation analysis between binding titers against specific antigens and binding titers against mature AdV-7 virions. FIGS. 15G-15I. Correlation analysis demonstrating the relationships between binding titers against specific antigens. In FIGS. 15D-15I, data were analyzed by two-tailed Pearson's correlation analysis, alpha=0.05. R² and p-values are presented for each correlation.

FIGS. 16A-16F. Assessment of immunogenicity of AdVLP-7 by neutralization assay. Neutralizing antibody titers in sera collected from AdVLP-7-immunized mice, WT AdV-7-immunized mice, and mice injected with a sham (buffer) were measured using a microneutralization assay based on a recombinant AdV-7 that expresses GFP. FIG. 16A. Representative images of A549 cells taken 28 hours after infection with rAdV-7 that had been mixed with serum from a sample animal in each group at a 1/250 dilution. Scale bars are 200 μm. FIG. 16B. Neutralizing antibody titers, presented as the serum dilution at which half of the rAdV-7 was neutralized as compared to serum-negative controls (ID₅₀). ID₅₀ values for individual animals are presented as either circles (•) for females or triangles (▾) for males. Dotted line indicates lower limit of detection. In FIG. 16B, data are mean+SD. Results were analyzed by one-way ANOVA with Tukey's multiple comparisons test, with p<0.05 considered statistically significant. **** p<0.0001. FIGS. 16C-16F. Correlation analysis between neutralizing antibody titers and binding antibody titers against (FIG. 16C) AdV-7, (FIG. 16D) hexon, (FIG. 16E) penton, or (FIG. 16F) fiber (as determined by ELISA, see FIGS. 14A-D and FIGS. 15A-15I). In FIGS. 16C-16F, data were analyzed by two-tailed Pearson's correlation analysis, alpha=0.05. R² and p-values are presented for each correlation.

FIGS. 17A-17B. FIG. 17A: Overview illustration of the human adenovirus 5 (HAdV5) hexon hyper variable regions (HVR 1-7), which shows the size in amino acids of each HVR loop. These loops size can vary in different adenovirus types. The largest loops (e.g., HVR 1, 5 and 7 are located on top of the outer capsid, while HVR 2, 3, 4 and 6 are settled lower in the base. FIG. 17B: Schematic of the replacement of the hexon HVR 5 with alternative portions of the SARS-CoV-2 spike protein receptor binding domain (RBD) or the receptor binding motif (RBM). Similar swapping can be performed in the HVR 1, 2, 3, 4 and 7 alone or in combination.

FIG. 18. Western Blot analysis of the expression of the chimeric hexon protein (AdV-7) containing either the spike protein receptor binding domain (RBD, 193aa-red dots) or the receptor binding motif (RBM, 69aa-blue dots) of SARS-CoV-2. HEK293 cells were transfected with either a plasmids expressing the RBM/Hexon-100k or RBD/Hexon-100k. Cell lysates were prepared 72 hours post transfection, clarified and analyzed by Western blot using an anti-hexon as primary antibody. The estimated molecular weight of the hexon/RBD (red dots) and the hexon/RBM (blue dots) are 127 kDa and 112 kDa, respectively.

### DETAILED DESCRIPTION

The present application relates to recombinant adenovirus capsids or adenovirus-like particles (AdVLPs), compositions comprising AdVLPs, and methods of making and using these AdVLPs, including the creation and production of homologous and heterologous AdVLP based vaccines as well as their use for drug (small molecule) and macromolecule delivery including, but not limited to, nucleic acids and genome editing systems, cancer therapy and immunotherapy and diagnostic and therapeutic applications.

In particular, the present disclosure includes strategies and methods used for the development of novel monovalent, or multivalent vaccines that are able to protect humans or other species against infection with one or more adenovirus types or serotypes included, displayed, or produced by the vaccine. Also described herein are production methods that produce AdVLPs that display certain antigenic configurations. In one or more embodiments, these VLPs feature hybrid (chimeric), modified (mutated, truncated) or reengineered antigens relevant for the generation of broad, robust and durable immune responses including high levels of neutralizing antibodies able to protect against more than one adenovirus type or serotype. Single particle monovalent, bivalent or multivalent as well as reengineered or modified AdVLPs are assembled or combined and used to formulate vaccine compositions, which allow for the immunization and subsequent protection against one or more types, serotypes or antigenically distinct adenovirus types (e.g., Mastadenvirus genus, human adenovirus species B types 7, 11, 14, 21, 55 and species C type 4; Aviadenovirus genus, fowl adenovirus 1, 4, 9, or similar combinations of members of other genera).

AdVLP vaccines can be produced in suspension culture of eukaryotic cells from where they are obtained. After purification, concentration and formulation the vaccine can be administered by any suitable route, for example via either mucosal or parenteral routes, and induce an immune response able to protect against any or all of the adenovirus types, serotypes or distinct antigenic variants for which the vaccine was designed and formulated to protect.

In one or more embodiments, AdVLPs can be comprised of combinations of adenoviruses and other antigens (e.g., other human respiratory viruses such as influenza, coronaviruses or viruses infecting other species such avian, bovine, ovine and porcine pathogens that can be combined in vaccine formulations with species specific AdVLPs). In one or more embodiments of the present application, methods of providing immune response to additional viruses are also described.

Because the assembled structures and its derivatives can interact with specific cell surface receptors and exert distinct effects including cellular uptake, they may allow for use in various areas such as therapy, gene therapy, immunotherapy, genome editing, and cancer treatment, for example.

### Definitions

As used in this specification and the appended claims, the singular forms “a,” “an” and “the” include plural references unless the content clearly dictates otherwise. Thus, for example, reference to “a VLP” (or an “AdVLP”) can include a mixture of two or more such VLPs (or AdVLPs).

As used herein the term “adjuvant” refers to a compound that, when used in combination with a specific immunogen (e.g., an AdVLP) in a formulation, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses.

An “antigen” refers to a molecule containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a cytotoxic T lymphocyte (CTL) epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts which produce the antigens.

As use herein, the term “antigenic formulation” or “antigenic composition” refers to a preparation which, when administered to a vertebrate, e.g., a mammal, will induce an immune response.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

As used herein an “effective dose” generally refers to that amount of VLPs (e.g., AdVLPs) of the present application sufficient to induce immunity, to prevent and/or ameliorate an infection or to reduce at least one symptom of an infection and/or to enhance the efficacy of another dose of a VLP. An effective dose may refer to the amount of VLPs sufficient to delay or minimize the onset of an infection. An effective dose may also refer to the amount of VLPs that provides a therapeutic benefit in the treatment or management of an infection. Further, an effective dose is the amount with respect to VLPs of the invention alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

As used herein, the term “effective amount” refers to an amount of VLPs (e.g., AdVLPs) necessary or sufficient to realize a desired biologic effect. An effective amount of the composition would be the amount that achieves a selected result, and such an amount can be determined as a matter of routine experimentation by a person skilled in the art. For example, an effective amount for preventing, treating and/or ameliorating an infection can be the amount necessary to cause activation of the immune system, resulting in the development of an antigen specific immune response upon exposure to VLPs of the invention. The term is also synonymous with “sufficient amount.”

An “immunogenic composition” is a composition that comprises an antigenic molecule where administration of the composition to a subject results in the development in the subject of a humoral and/or a cellular immune response to the antigenic molecule of interest.

An “immunological response” or “immune response” to an antigen or composition is the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytotoxic T lymphocytes (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of suppressor T-cells and/or γΔ T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

As used herein, the term “multivalent” refers to VLPs (e.g., AdVLPs) which have multiple antigenic proteins against multiple types or strains of infectious agents or alternative conformations of the same antigen/protein (metastable), which naturally transition from one conformation to the next, but in the context of a vaccine formulation may contain stabilized (fixed) form of one conformation or both.

A “nucleic acid” molecule can include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also captures sequences that include any of the known base analogs of DNA and RNA.

By “pharmaceutically acceptable” or “pharmacologically acceptable” is meant a material which is not biologically or otherwise undesirable, i.e., the material may be administered to an individual in a formulation or composition without causing any unacceptable biological effects or interacting in a deleterious manner with any of the components of the composition in which it is contained.

As used herein the term “protective immunity”, “protective immune response” or “protective response” refers to an immune response mediated by antibodies against an infectious agent, which is exhibited by a vertebrate (e.g., a human), that prevents or ameliorates an infection or reduces at least one symptom thereof. VLPs of the present application can stimulate the production of antibodies that, for example, neutralize infectious agents, blocks infectious agents from entering cells, blocks replication of said infectious agents, and/or protect host cells from infection and destruction. The term can also refer to an immune response that is mediated by T-lymphocytes and/or other white blood cells against an infectious agent, exhibited by a vertebrate (e.g., a human), that prevents or ameliorates adenovirus infection or reduces at least one symptom thereof.

“Purified” or “Substantially purified” general refers to isolation of a substance (compound, polynucleotide, protein, polypeptide, polypeptide composition) such that the substance comprises the majority percent of the sample in which it resides. Typically, in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

“Recombinant” as used herein to describe a nucleic acid molecule means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

As used herein, the term “spike receptor binding domain” (RBD) refers to a part of a virus located on its ‘spike’ domain that allows it to dock to body receptors to gain entry into cells.

By “subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

As used herein, “treatment” refers to any of (i) the prevention of infection or reinfection, as in a traditional vaccine, (ii) the reduction or elimination of symptoms, and (iii) the substantial or complete elimination of the pathogen in question. Treatment may be effected prophylactically (prior to infection) or therapeutically (following infection).

As used herein, the term “vaccine” refers to a formulation which contains VLPs (e.g., AdVLPs) of the present application, which is in a form that is capable of being administered to a vertebrate and which induces a protective immune response sufficient to induce immunity to prevent and/or ameliorate an infection and/or to reduce at least one symptom of an infection and/or to enhance the efficacy of another dose of VLPs. Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

A “vector” is capable of transferring gene sequences to target cells (e.g., bacterial plasmid vectors, viral vectors, non-viral vectors, particulate carriers, and liposomes). Typically, “vector construct,” “expression vector,” and “gene transfer vector,” mean any nucleic acid construct capable of directing the expression of one or more sequences of interest in a host cell. Thus, the term includes cloning and expression vehicles, as well as viral vectors. The vector is usually a plasmid designed for gene expression in cells. The term is used interchangeably with the terms “nucleic acid expression vector”, “expression plasmid”, and “expression cassette”.

As used herein, the terms “virus-like particle”, “VLP”, “recombinant virus-like particle” or “recombinant VLP” (e.g., AdVLP) refer to a nonreplicating, viral shell. VLPs are generally composed of one or more viral proteins, such as, but not limited to those proteins referred to as capsid, coat, shell, surface and/or envelope proteins, or particle-forming polypeptides derived from these proteins. VLPs can also be described as “enveloped” if they contain a cell derived lipid membrane or non-enveloped if assembly with protein without a lipid membrane. The terms nanoparticles or nanospheres have also been used to described virus particles which do not defer in composition from the virus like particles delineated in this disclosure. VLPs can form spontaneously upon recombinant expression of the protein in an appropriate expression system. Methods for producing particular VLPs are known in the art and discussed more fully below. The presence of VLPs following recombinant expression of viral proteins can be detected using conventional techniques known in the art, such as by electron microscopy, biophysical and immunological characterizations, and the like. See, e.g., Baker et al., Biophys. J. (1991) 60:1445-1456; Hagensee et al., J. Virol. (1994) 68:4503-4505. For example, VLPs can be isolated by density gradient centrifugation and/or identified by characteristic density banding. Alternatively, cryoelectron microscopy can be performed on vitrified aqueous samples of the VLP preparation in question, and images recorded under appropriate exposure conditions. Additional methods of VLP purification include, but are not limited to, chromatographic techniques such as affinity, ion exchange, size exclusion, and reverse phase procedures.

As used herein, the term “about” or “approximately” for a numerical value means ±3% of the numerical value.

### Virus-Like Particles (VLPs)

VLPs²⁹ are structurally similar to native viruses, but are completely devoid of genetic material, rendering them non-infectious. This combination allows antigens to be presented to the immune system in their native conformation without the risk of vaccine-associated viral shedding or recombination. VLPs have been safely and successfully applied as vaccine platforms against human papillomavirus, hepatitis B virus, and hepatitis E virus³⁰⁻³⁴. Self-assembly of VLPs is typically driven by recombinant expression of viral structural proteins in mammalian, bacterial, yeast, or plant-based expression systems³⁵. In AdVs, the bulk of the capsid is composed of major capsid proteins (hexon, penton, and fiber), which are structurally supported by the minor capsid/cement proteins (IIIa, VI, VIII, and IX)^{36,37}. The major capsid proteins, specifically hexon, are the primary target of neutralizing antibodies³⁸⁻⁴¹, which are associated with protection against disease^{42,43}. The VLP platform of the present application is an effective alternative to the existing live virus Adenovirus vaccines for administration to both military recruits and to the general public, for example.

Furthermore, VLPs can also be used for the diagnosis of infection or for therapeutic indications. VLP vaccines can be produced via transient transfection of suspension culture of eukaryotic cells or suspension culture of stably transfected cells that constitutively produce the VLPs, which are released into the culture medium. After purification, concentration, and formulation, the vaccine can be administered by any suitable route, for example, via either mucosal or parenteral routes, and induce an immune response able to protect against any or all coronaviruses, antigenic variants, etc. VLPs comprising therapeutics, immunomodulatory functions and diagnostic application are also provided.

### Adenoviruses

Virion Structure: Adenoviruses (AdVs) are non-enveloped icosahedral particles of approximately 90 nm in diameter, which do not contain either membranes or lipids. This icosahedral capsid is composed predominantly of proteins and encases a linear double-stranded DNA (dsDNA) genome-containing core. The AdV capsid is constructed by the assembly of 14 proteins including the hexon (protein II of 109 kDa in size), the penton base (protein III of 63.3 kDa in size) and the fiber (protein IV of 38-61.1 kDa in size), which are referred to as the major capsid proteins. Twelve copies of these hexon trimers form each one the 20 facets of the icosahedron that represents the most abundant capsid protein component. Each of the 12 vertices of the icosahedron is occupied by a pentameric penton base, which forms a complex with a trimer of the protruding fiber. The fiber trimers assemble into a shaft that extends outward from the center of the pentons and terminates forming a knob structure, which serves as the adenoviruses' attachment protein to cell surface receptors during infection. The minor capsid components include protein IIIa (63.5 kDa), VIII (24.0 kDa) and IX (14.4 kDa) that function as cement proteins linking the major structural building blocks with each other and the viral core. Protein VI (27 kDa) is also a cement protein that plays additional roles in virion maturation, entry, trafficking and early gene expression. The viral core is composed of the dsDNA genome (˜36Kbp that varies based on virus type and genus) that is associated with four proteins: V (41.6 kDa), VII (19.4 kDa), μ (4 kDa) and terminal protein (TP-55 kDa), in addition to the viral protease (23 kDa). Furthermore, other viral-encoded proteins present in infected cells seem also to participate in capsid assembly and/or genome packaging such as proteins IVa2, L4 33K, L4 22K, E2 72K and L1-52/55K.

AdV morphogenesis appears to follow a sequential pathway that involves numerous consecutive steps. The capsid formation begins with the assembly of hexon and penton capsomers followed by the assembly of empty capsids and also uses the minor capsid cement proteins and involvement of non-structural proteins. Assembly of the hexon trimers depends upon a virus-encoded protein, L4 100K, that serves as a chaperone-like element guiding hexon trimerization. Subsequent to capsid assembly, packaging proteins recognize the packaging domain in the viral genome and initiate its insertion into the empty capsid, likely through a single opening located in one vertex of the structure. Completion of the process entails cleavage of some of the capsid components by the viral protease and release of scaffolding and some packaging proteins allowing for the maturation of viral particles.

Adenoviruses enter target cells via a receptor-mediated endocytosis mechanism. During the initial step the fiber protein, which protrudes from each vertex of the icosahedron and terminates as a globular head (the knob), attaches with high affinity to cell surface receptors. Adenoviruses from species A, C through G attach to the coxsackie and adenovirus receptor (CAR) whereas members of the B species use CD46 and desmoglein-2 (DSG-2) as their primary receptor. Following attachment, virus uptake is mediated by the binding of the penton base protein with the αvβ3/αvβ5 integrins, which enable cell uptake and initiation of infection. Some other cell surface molecules have also been described as co-receptors for adenovirus such as MHC class I, sialic acid, and coagulation factor X. After infection, the adenovirus completes its replication cycle inside the cells.

Human adenoviruses are known to cause respiratory infections, conjunctivitis and gastroenteritis in normal individuals. In neonates and immunosuppressed individuals, however, adenovirus can cause serious illness and fulminant fatal pneumonia, hepatitis or encephalitis. Human adenovirus infections account for a small portion of acute respiratory illnesses in the general population and for about 5% to 10% of respiratory ailments in children, however, they can cause serious respiratory epidemic outbreaks amongst military recruits and in nursing homes. A live adenovirus vaccine formulated with human adenovirus types 4 and 7, which are the most prevalent serotypes causing disease in military recruits, is currently in use in military centers. These live viruses are enclosed in an enteric-coated pill and administered via the oral route. It is likely that due to only serotype-specific immunity offered by current vaccines and in consideration of the fact that many human adenoviruses are able to cause significant respiratory illness, it is distinctly possible that many are at risk of serious illness from multiple strains of the disease. Furthermore, virus recombination events may result in new antigenic serotypes, which may further increase the rate of respiratory epidemic outbreaks.

To address the limitations of the existing live adenovirus vaccine, the present application, in accordance with one or more embodiments, provides a new generation of adenovirus vaccines that can not only protect against multiple adenovirus types, but also, in certain embodiments, can be upgraded to include emerging serotypes. Safe non-replicating vaccines such as the ones described in this application can be used to prevent outbreaks in crowded settings such as nursing home, military facilities, etc. as well as to prevent disease in the general populations.

Protection provided by the AdVLP vaccines of the present application against adenovirus infection is primarily mediated by neutralizing antibodies targeting the major capsid proteins, particularly the most abundant hexon polypeptide that is both a type-specific and species-specific antigen. In addition, neutralizing antibodies that result from immunization with the proposed vaccine target the fiber protein and contribute to virus neutralization by synergizing with antibodies directed to the penton base, the latter of which mediate binding with the secondary HAdV receptor.

The external surface of the hexon protein exhibits loops with hypervariable sequences that are significant for adenovirus type-specific immunogenicity and elicitation of protective neutralizing antibodies. The ability of these domains to tolerate a significant amount of variability likely aids in evasion of the immune response. These hexon loops, however, are suitable for modifications or grafting of antigenic domains from related or unrelated sources.

### Adenovirus Virus-Like Particles (AdVLPs) and Corresponding Plasmids, Methods, Compositions and Uses Thereof

In accordance with one or more embodiments, provided herein are adenovirus virus-like particles (AdVLPs). AdVLPs are recombinant capsids or shells that comprises capsid proteins of one or more adenoviruses. In particular, in one or more embodiments, the AdVLP is comprised of a recombinant capsid that includes major capsid adenovirus (AdV) proteins, specifically a hexon protein, a penton protein, and a fiber protein. The recombinant capsid of the AdVLP further includes minor capsid/cement AdV proteins, including a IIIa protein, a VI protein, a VIII protein, and a IX protein. The minor capsid/cement AdV proteins structurally support the major capsid AdV proteins in the recombinant capsid. The recombinant capsid further includes a chaperone AdV protein L4-100k, and an accessory scaffold AdV protein L1-52/55k. The major and minor capsid proteins, along with the chaperone and accessory scaffold proteins assemble into icosahedral (recombinant) capsids to form the AdVLPs, which are analogous to the empty capsids present in adenovirus-infected cells. In one or more embodiments, these structures appear to be primarily composed of 240 capsomeres of hexon trimers (12 per each triangular facet of the icosahedron), 12 pentameric penton capsomeres each occupying a vertex of the 12 vertices of the icosahedron and 12 trimeric fibers each projecting outward from the pentons. Exemplary amino acid and nucleotide sequences of various major and minor capsid proteins, and chaperone and accessory scaffold proteins of exemplary AdVLPs of the present application are provided following the examples section.

In one or more embodiments, the AdVLPs of the present application are formed from major and minor capsid proteins, chaperone proteins, and accessory scaffold proteins of one or more types of adenoviruses, including but not limited to: human adenovirus-A (HAdV-A) type: 12, 18, or 31; HAdV-B type: 3, 7,11,14,16, 21, 34, 35, 50, or 55; HAdV-C type: 1, 2, 5, or 6; HAdV-D type: 8-10, 13,15,17,19, 20, 22-30, 32, 33, 36-39, 42-49, 51, 53, or 54; HAdV-E type: 4; HAdV-F type: 40 or 41, and HAdV-G type: 52. In at least one embodiment, proteins from genera other than human adenovirus, such as the Aviadenovirus genus that include viruses infecting avian species, can be utilized to form the AdVLPs. In one or more embodiments, the AdVLPs of the present application are formed from major and minor capsid proteins, chaperone proteins, and accessory scaffold proteins of one or more of HAdV-E type 4 (AdVLP4), HAdV-B type 7 (AdVLP7), HAdV-B type 14 (AdVLP14), and HAdV-B type 55 (AdVLP55).

In one or more embodiments, the AdVLP can comprises hexon proteins from different serotypes (e.g., two different adenovirus serotypes) such that the AdVLP is chimeric.

In one or more embodiments, the AdVLP can further include one or more adjuvants. For example, in one or more embodiments, the adjuvant can be aluminum hydroxide (Alum) or a squalene-based oil-in-water nano-emulsion (e.g., ADDAVAX (InvivoGen, San Diego, CA, USA)).

The AdVLPs and compositions of the present application can be administered to a subject by any mode of delivery, including, for example, by parenteral injection (e.g., subcutaneously, intraperitoneally, intravenously, intramuscularly, or to the interstitial space of a tissue), or by rectal, oral (e.g., tablet, spray), vaginal, topical, transdermal (e.g., see International Publication No. WO99/27961) or transcutaneous (e.g., see International Publication Nos. WO02/074244 and WO02/064162), intranasal (e.g., see International Publication No. WO03/028760), ocular, aural, pulmonary or other mucosal administration and/or inhalation of powder compositions. Multiple doses can be administered by the same or different routes.

The AdVLPs (and AdVLP-containing compositions) can be administered prior to, concurrent with, or subsequent to delivery of other vaccines, for example. Additionally, the site of AdVLP administration may be the same or different as other vaccine compositions that are being administered.

Dosage treatment with the AdVLP composition may be a single dose schedule or a multiple dose schedule. A multiple dose schedule is one in which a primary course of vaccination may be with 1-10 separate doses, followed by other doses given at subsequent time intervals, chosen to maintain and/or reinforce the immune response, for example at 1-4 months for a second dose, and if needed, a subsequent dose(s) after several months. The dosage regimen will also, at least in part, be determined by the potency of the modality, the vaccine delivery employed, the need of the subject and be dependent on the judgment of the practitioner.

In one or more embodiments, an expression plasmid is provided comprising genes encoding adenovirus proteins, such that the expression plasmid is suitable for the assembly of the adenovirus virus-like particles (AdVLPs) of the present application. The expression plasmid can comprise codon-optimized genes that encode for major capsid adenovirus (AdV) proteins (hexon, penton, and fiber), the minor capsid/cement AdV proteins (IIIa, VI, VIII, and IX protein), a chaperone AdV protein L4-100k, and an accessory scaffold AdV protein L1-52/55k. In one or more embodiments, the codon-optimized genes encode for proteins of one or more types of adenoviruses such as HAdV-A type, B type, C type, D type, E type, F type, and G type adenoviruses as described above, or other types of adenoviruses. For instance, in at least one embodiment, the genes encode for proteins of one or more of AdVLP4, AdVLP7, AdVLP14, and AdVLP55.

In accordance with one or more embodiments, an immunogenic composition (e.g., vaccine) is provided that comprises at least one AdVLP of the present application (e.g., AdVLP4, AdVLP7, AdVLP14, AdVLP55). In one or more embodiments, the immunogenic composition can be monovalent, bivalent, trivalent, quadrivalent, polyvalent or multivalent, such that the immunogenic composition can be attached to one type of antigen (monovalent), two types of antigens (bivalent), three types of antigens (trivalent), etc., wherein the different antigens can be different types of adenoviruses, as described above. Thus, in one or more embodiments, the immunogenic composition can immunize a subject (e.g., human) against one or more types of adenoviruses.

In at least one embodiment, the immunogenic composition is a trivalent composition comprising AdVLP4, AdVLP7, and AdVLP14 (such that it immunizes a subject against HAdV-E type 4, HAdV-B type 7, and HAdV-B type 14). In at least one embodiment, the immunogenic composition is a quadrivalent composition comprising AdVLP4, AdVLP7, AdVLP14, and AdVLP55 (such that it immunizes a subject against HAdV-E type 4, HAdV-B type 7, HAdV-B type 14, and HAdV-B type 55).

In one or more embodiments, a carrier is optionally present in the compositions (e.g., immunogenic compositions, AdVLPs) described herein. Typically, a carrier is a molecule that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; McGee J P, et al., J Microencapsul. 14(2):197-210, 1997; O'Hagan D T, et al., Vaccine 11(2):149-54, 1993. Such carriers are well known to those of ordinary skill in the art.

Additionally, these carriers may function as immunostimulating agents (“adjuvants”). Exemplary adjuvants include, but are not limited to: (1) aluminum salts (alum), such as aluminum hydroxide, aluminum phosphate, aluminum sulfate, etc.; (2) oil-in-water emulsion formulations (with or without other specific immunostimulating agents such as muramyl peptides or bacterial cell wall components), such as for example (a) MF59 (International Publication No. WO 90/14837), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, Mass.), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribi™ adjuvant system (RAS), (Ribi Immunochem, Hamilton, MT) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detoxu), (d) ADDAVAX™ vaccine adjuvant, a squalene-based oil-in-water nano-emulsion (InvivoGen, San Diego, CA, USA); (3) saponin adjuvants, such as Stimulon™. (Cambridge Bioscience, Worcester, Mass.) may be used or particles generated therefrom such as ISCOMs (immunostimulating complexes); (4) Complete Freunds Adjuvant (CFA) and Incomplete Freunds Adjuvant (IFA); (5) cytokines, such as interleukins (IL-I, IL-2, etc.), macrophage colony stimulating factor (M-CSF), tumor necrosis factor (TNF), beta chemokines (MIP, 1-alpha, 1-beta Rantes, etc.); (6) detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63), LT-R72 (where arginine is substituted for the wild-type amino acid at position 72), CT-S109 (where serine is substituted for the wild-type amino acid at position 109), and PT-K9/G129 (where lysine is substituted for the wild-type amino acid at position 9 and glycine substituted at position 129) (see, e.g., International Publication Nos. WO 93/13202 and WO 92/19265); and (7) other substances that act as immunostimulating agents to enhance the effectiveness of the composition.

In at least one embodiment, a method of generating an immune response to one or more adenoviruses in a subject is provided. The method comprises administering an effective amount of the immunogenic composition to the subject. In one or more embodiments, the immunogenic composition can be administered by oral, nasal, mucosal, or parenteral administration, or other mode of administration, as described above. In one or more embodiments of the method, the one or more adenovirsues are selected from the group consisting of HAdV-E type 4, HAdV-B type 7, HAdV-B type 14, and HAdV-B type 55, and in one or more embodiments, the immune response vaccinates the subject (e.g., human) against the one or more adenoviruses.

As such, the immunogenic compositions of the present application (e.g., AdVLP-based vaccines) can trigger, upon human or other species administration, a strong and balanced immune response characterized by the induction of high levels of neutralizing antibodies against one or more adenovirus species, types, serotypes or antigenic variants.

In one or more embodiments, the immunogenic composition may induce a humoral immune response in the subject administered the immunogenic composition. In some embodiments, the induced humoral immune response may be specific for one or more adenoviruses. The humoral immune response may be induced in the subject administered the immunogenic composition by about 1.5-fold to about 100-fold, about 2-fold to about 90-fold, or about 3-fold to about 80-fold. The humoral immune response can be induced in the subject administered the immunogenic composition by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 20-fold, at least about 30-fold, at least about 40-fold, at least about 50-fold, at least about 60-fold, at least about 70-fold, at least about 80-fold, at least about 90-fold, at least about 100-fold, or more. The humoral immune response induced by the immunogenic composition may include an increased level of neutralizing antibodies associated with the subject administered the immunogenic composition as compared to a subject that is not administered the immunogenic composition. The neutralizing antibodies may be specific for one or more adenoviruses. The neutralizing antibodies can provide protection against and/or treatment of infections from one or more adenoviruses and their associated pathologies in the subject administered the immunogenic composition.

In one or more embodiments, the humoral immune response induced by the immunogenic composition may include an increased level of IgG antibodies associated with the subject administered the immunogenic composition as compared to a subject not administered the immunogenic composition.

In at least one embodiment, the humoral response may be cross-reactive against two or more strains or types of adenovirus. The level of IgG antibody associated with the subject administered the immunogenic composition may be increased by about 1.5-fold to about 100-fold, about 2-fold to about 50-fold, or about 3-fold to about 25-fold as compared to the subject not administered the immunogenic composition. The level of IgG antibody associated with the subject administered the immunogenic composition can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 20-fold, at least about 30-fold, at least about 40-fold, at least about 50-fold, at least about 60-fold, at least about 70-fold, at least about 80-fold, at least about 90-fold, at least about 100-fold, or more.

An appropriate effective amount of the immunogenic composition for administration to the subject can be determined by one of skill in the art. Such an amount will fall in a relatively broad range that can be determined through routine trials and will generally be an amount on the order of about 0.1 μg to about 10 (or more) mg, more preferably about 1 μg to about 300 μg, of VLP/antigen. In another aspect, the immune response vaccinates the subject against one or more coronaviruses. For instance, in at least one embodiment, the immune response vaccinates the subject against one or more adenoviruses.

In accordance with one or more embodiments, a method of producing an AdVLP is provided. The method of producing the AdVLP includes introducing into a host cell at least one expression plasmid under conditions such that the host cell produces the AdVLP. The at least one expression plasmid introduced into the host cell comprises codon-optimized genes encoding: a) major capsid adenovirus (AdV) proteins (hexon, penton, fiber); b) minor capsid/cement AdV proteins (IIIa, VI, VIII, and IX); c) a chaperone AdV protein L4-100k; and d) an accessory scaffold AdV protein L1-52/55k. In one or more embodiments, the host cell is a eukaryotic cell, for example, a mammalian cell.

In at least one embodiment, the method of producing the AdVLP can further include a step of purifying the AdVLP. The AdVLP can be purified from cell lysates and culture supernatants, as exemplified in the example section below. It should be understood, however, that in other embodiments, the AdVLP can be purified using other approaches as is known and understood in the art.

The strategies for adenovirus vaccine development of the present application are based on the recombinant expression of adenovirus-like particles (AdVLPs) composed of alternative combinations of the structural elements involved in the morphogenesis of complete icosahedral virion particles. AdVLPs lack a viral genome and therefore are unable to replicate or cause infection thereby serving as suitable candidates for vaccine development. Lack of infectivity of AdVLP precludes the need for chemical inactivation, better preserving the immunological attributes of a vaccine based on this approach. Furthermore, stimulation of immunity by AdVLP is independent of viral replication avoiding viral interference caused by the replication dominance of one virus over the others in the composition. This may lead to an imbalance of the immune response when multiple replicating viral strains are combined in a live vaccine.

### Application and Uses of the Adenovirus Virus-Like Particles (AdVLP) as Delivery Vehicles, Vectors, Carriers

In accordance with one or more embodiments, the AdVLPs of the present application can provide utility as vaccines, delivery vehicles, therapeutics, drug enhancers, or diagnostic tools. For instance, in one or more embodiments, the AdVLPs of the present application can be used for a diverse range of applications because as biocompatible biological nanoparticles (e.g., AdVLP size ˜90 nm), they may serve as carriers of peptides, nucleic acids, glycans, and small molecules, etc. Furthermore, they can be modified, via genetic or chemical methods, to display homologous or heterologous proteins, antigens or peptide sequences and through bioconjugation, be able to display diverse molecular entities such as glycans, proteins, lipids, drugs, etc.

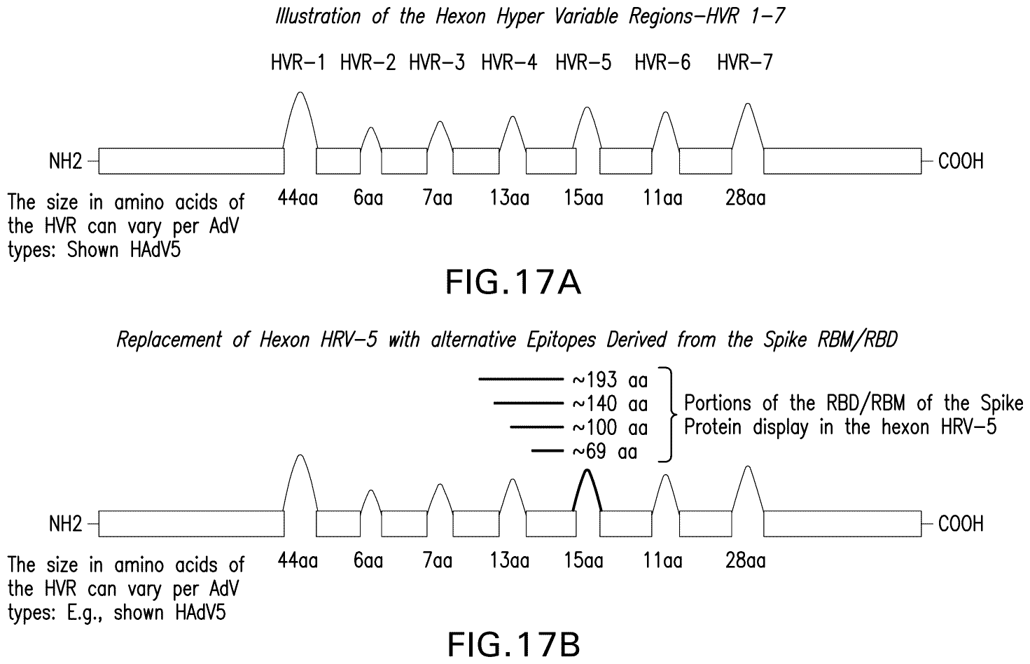

These various AdVLP designs can allow for their use in numerous fields such as for vaccines, imaging, targeted and non-targeted therapeutics, gene therapies, cancer vaccines and therapies, immunomodulation agents, nanodevices, among others. For instance, the hypervariable region (HVR) of the hexon protein, the Arg-Gly-Asp (RGD) domain loop of the penton base protein, the carboxyl terminal and HI loop of the fiber knob and the carboxyl terminal of the protein IX in addition to other possible sites may be suitable for genetic modifications or chemical conjugation of different molecular entities. For example, FIG. 17A provides an overview illustration of an exemplary human adenovirus 5 (HAdV5) hexon hyper variable regions (HVR 1-7), which shows the size in amino acids of each HVR loop (part A). These loops size can vary in different adenovirus types. The largest loops (e.g., HVR 1, 5 and 7 are located on top of the outer capsid, while HVR 2, 3, 4 and 6 are settled lower in the base. FIG. 17B shows a schematic of the replacement of the hexon HVR 5 with alternative portions of the SARS-CoV-2 spike protein receptor binding domain (RBD) or the receptor binding motif (RBM). Similar swapping can be performed in the HVR 1, 2, 3, 4 and 7 alone or in combination. These modifications allow for the display of heterologous antigenic determinants useful for prophylactic or therapeutic vaccines against infectious agents or cancer, for example. Furthermore, HVR sequence alternations may introduce changes suitable for the subsequent specific attachments of various molecular entities via conjugation, click-chemistry or analogous methods.

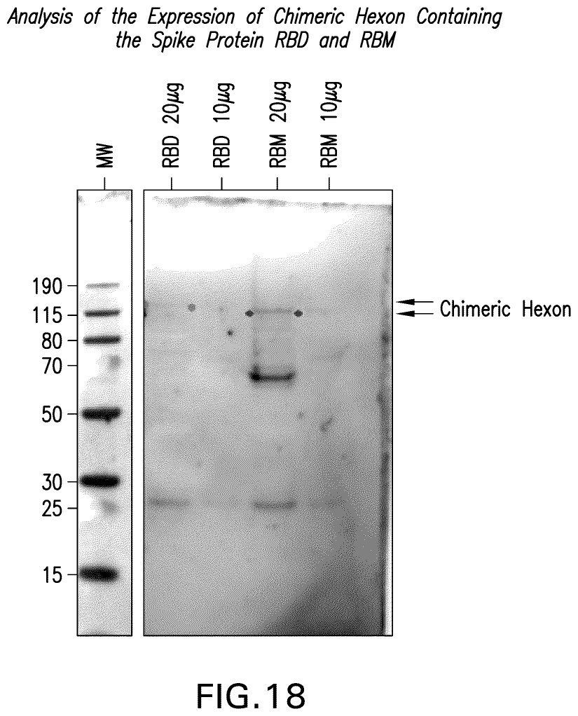

In one or more embodiments, the AdVLPs of the present application can feature hybrid (chimeric) antigens. For instance, in at least one embodiment, the hexon protein of the AdVLP can comprise one or more antigens of a different infectious agent (an infectious agent other than an adenovirus). For example, FIG. 18 displays a Western Blot analysis of the expression of the chimeric hexon protein (AdV-7) containing either the spike protein receptor binding domain (RBD, 193aa-red dots) or the receptor binding motif (RBM, 69aa-blue dots) of SARS-CoV-2. These chimeric constructs can be used as a vaccine for the prevention of SARS-CoV-2 infection. In this example, HEK293 cells were transfected with either plasmids expressing the RBM/Hexon-100k or RBD/Hexon-100k. Cell lysates were prepared 72 hours post transfection, clarified and analyzed by Western blot using an anti-hexon as primary antibody. The estimated molecular weight of the hexon/RBD (red dots) and the hexon/RBM (blue dots) are 127 kDa and 112 kDa, respectively.

In addition to the functionalization of the exterior or interior surfaces of the particles, encapsulation or incorporation of new material within the interior cavity of these particles can further expand their range of use. Different techniques can be deployed for the incorporation, packaging or entrapment of small molecules, proteins, nucleic acids, or other molecular entities. Furthermore, understanding of the adenovirus assembly pathways may guide the discovery of new antiviral drugs and further advance nanotechnological applications. Thus, the assembly of native or modified AdVLPs broaden their utility beyond the field of adenovirus vaccine use as described above.

Exemplary embodiments and applications of the AdVLPs of the present application are described in further detail in the Examples section below.

### EXAMPLES

### 1. Adenovirus Virus-Like Particle (AdVLP) Assembly, Production and Characterization

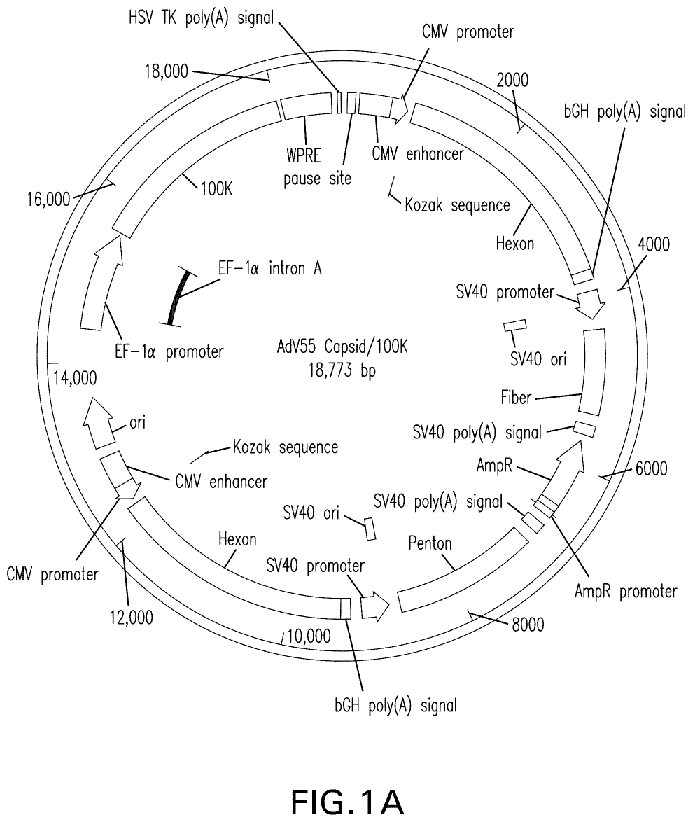

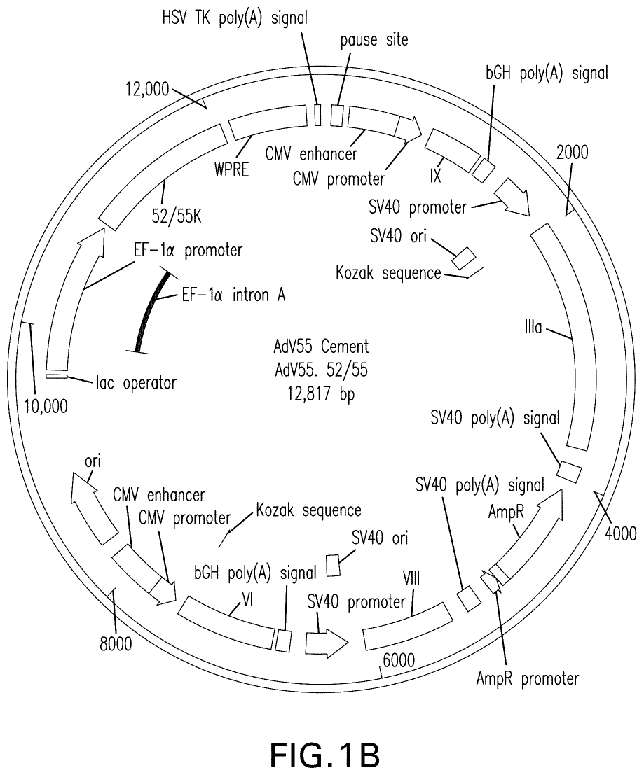

An exemplary methodology for the self-assembly of recombinant adenovirus capsids or adenovirus virus-like particles (AdVLPs) is described herein. These are structures which exist as empty capsids that lack the viral genome and found in adenovirus infected cells. To assemble and produce these structures, multi-gene expression plasmids are utilized which, following their transfection into mammalian cell suspension cultures, direct the production of the proteins needed for the self-assembly of recombinant adenovirus capsids or adenovirus-like particles (AdVLPs). In one instance, two quintuple expression plasmids are utilized, one carrying the adenovirus major capsid proteins, hexon (two copies), penton and fiber together with the chaperone 100K protein and a second plasmid carrying the genes encoding minor adenovirus capsid proteins or the cement proteins, e.g., IX, IIIa, VI and VIII, together with a gene encoding for the scaffold protein 52/55, in order to construct the unique AdVLP. An example of expression plasmids utilized for the production of the AdVLP55 (adenovirus type 55) are shown in FIGS. 1A and 1B. Similar constructs were also created for the production of AdVLP4, AdVLP7 and AdVLP14 (adenovirus type 4, 7, and 14).

Alternative methods for the production of AdVLPs include, but are not limited to, the use of differing numbers of plasmids, engineered cells for the stable production of some or all the needed proteins, modified cells that retain and amplify the expression plasmids for the prolong production of AdVLPs and other developed technology. Examples of additional or alternative methods use for the production and/or stabilization of the AdVLP particles may include other adenovirus genes such as IVa2, L4 22K, L4 33K, E2 72K and the viral protease (AVP-23 kDa) as well as the internal proteins, V (41.6 kDa), VII (19.9 kDa), and mu (4 kDa).

These AdVLPs can be purified from cell lysates and culture supernatants utilizing either cesium chloride (CsCl) gradient ultra-centrifugation methods, (e.g., discontinuous and isopycnic density gradients) or alternatively via tangential flow filtration and ionic-exchange chromatography.

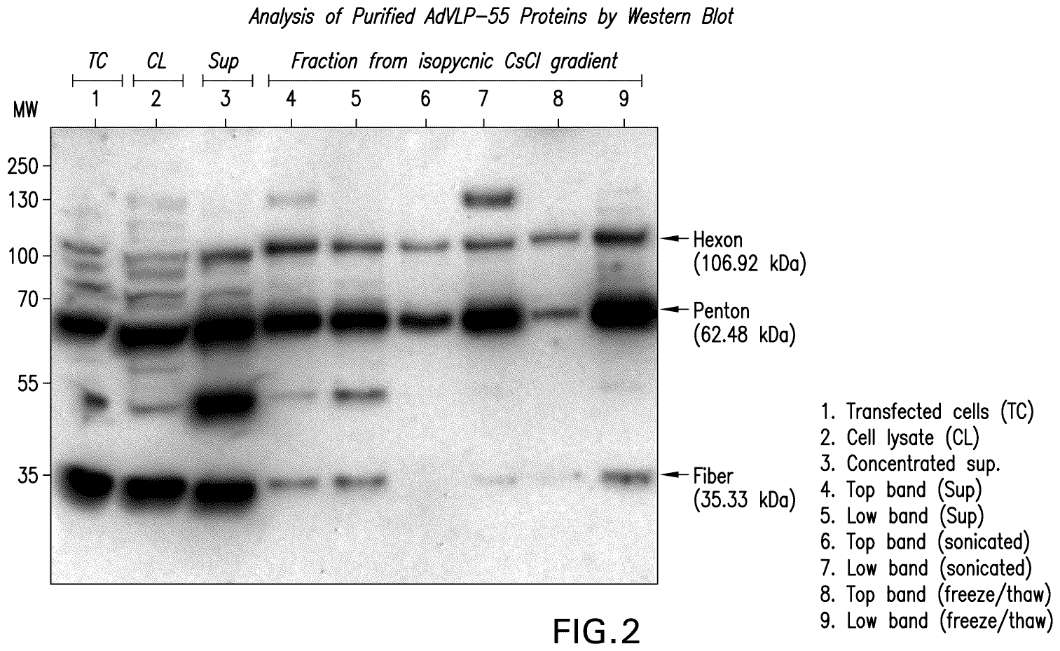

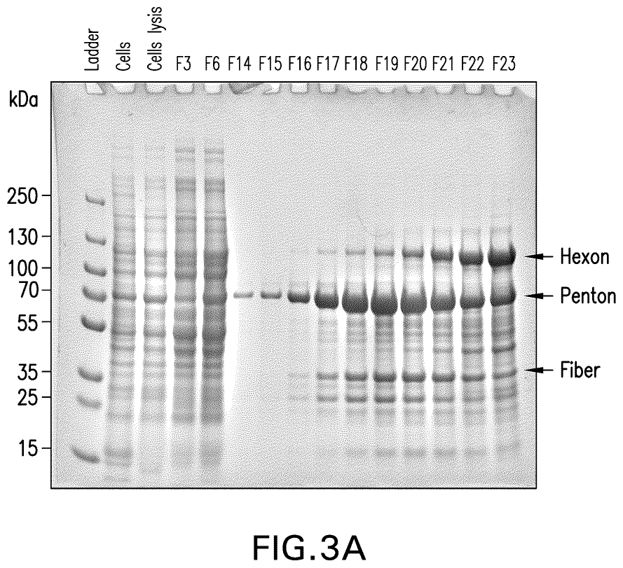

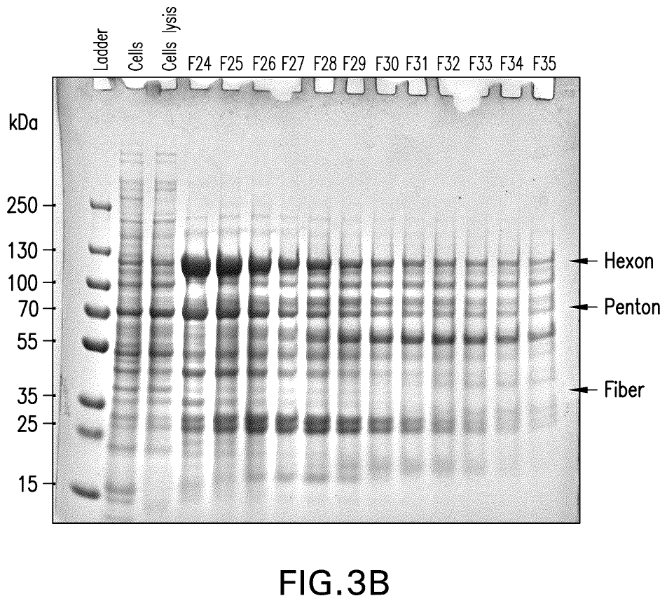

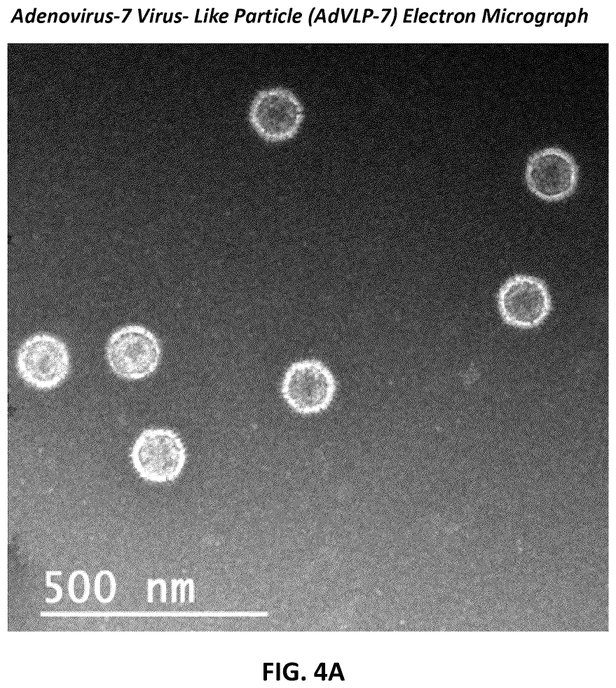

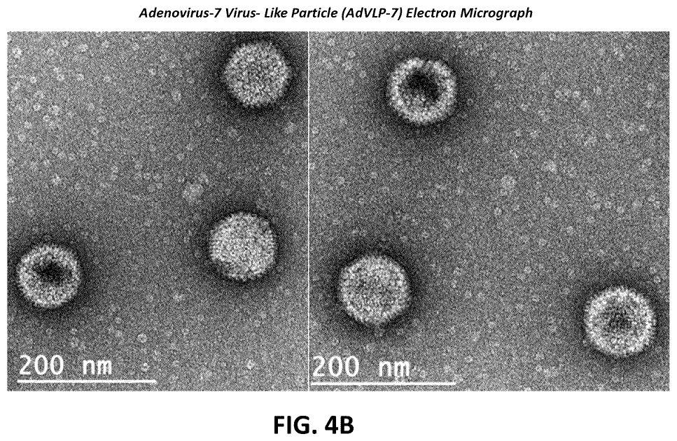

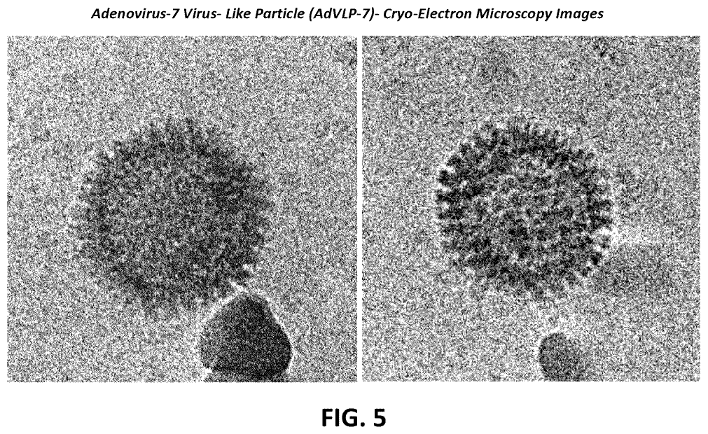

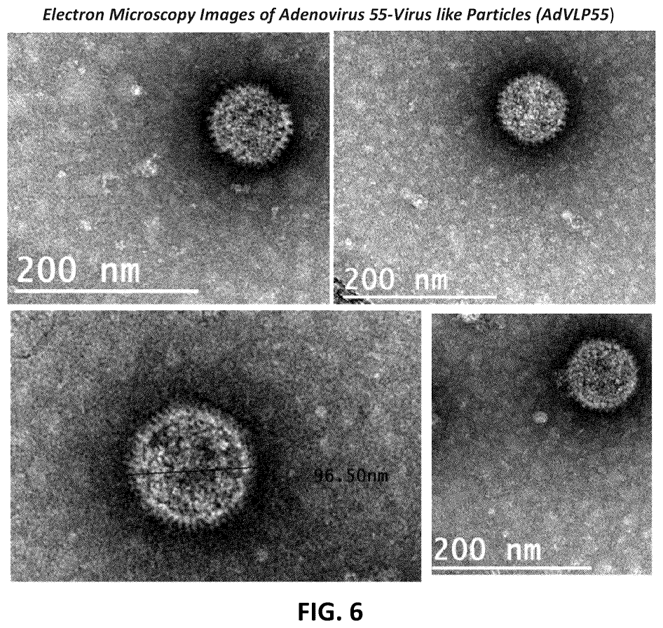

The protein content of the AdVLPs are analyzed via Western blot and Coomassie blue staining techniques. Examples of the results of a Western blot from a CsCl purified AdVLP55 and a Coomassie blue staining of an AdVLP55 purified via chromatography are shown in FIG. 2 and FIGS. 3A-3B, respectively. Further structural analysis of the AdVLPs are performed by examining samples via negative staining electron microscopy (EM) and cryo-electron microscopy. Examples of these analyses are shown in FIGS. 4A-4B, 5, and 6. These evaluations demonstrate that the assembled recombinant AdVLPs resemble the native adenovirus virion in size, morphology and overall architecture, confirming that the set of proteins utilized assemble into particles that resemble the structure of native adenovirus virions, FIGS. 4A-4B, 5, and 6.

Moreover, the utilization of a fewer number of structural proteins such as the pair of penton base and fiber alone may result in the assembly of dodecahedron particles, which represent much smaller and less complex structures than the complete empty capsids or mature capsids containing the viral genome. The recombinant capsids or shells described here named adenovirus-like particles (AdVLP) are composed of the major capsid proteins hexon, penton base and fiber which together with the minor capsid proteins or cement protein, IIIa, VI, VIII and IX and other accessory elements assemble into icosahedral capsids or AdVLPs that are analogous to the empty capsids present in adenovirus infected cells. These structures appear to be primarily composed of 240 capsomeres of hexon trimers (12 per each triangular facet of the icosahedron), 12 pentameric penton capsomeres each occupying a vertex of the 12 vertices of the icosahedron and 12 trimeric fibers each projecting outward from the pentons. During adenovirus infection, these structures seem to be preformed and then utilized for threading the genomic DNA into place within the capsid. In the present application, recombinant empty capsids (or AdVLPs as described), have been produced and their immunogenicity have been evaluated in animal models. Results from these studies showed that these structures elicited robust immune responses that can protect against adenovirus infection.

The strategy and methods described in this submission are applicable for the assembly and production of AdVLPs for any member of the Adenoviridae family as well as for viral families exhibiting capsid structures with essentially identical design to that seen with adenovirus such as bacteriophages PRD1 (family Tectiviridae), PM2 (family Corticoviridae), PBCV-1 (family Phycodnaviridae) as well as members of the Iridoviridae family.

### 2. Adenovirus Virus-Like Particles (AdVLP) as Monovalent or Polyvalent Vaccine Compositions

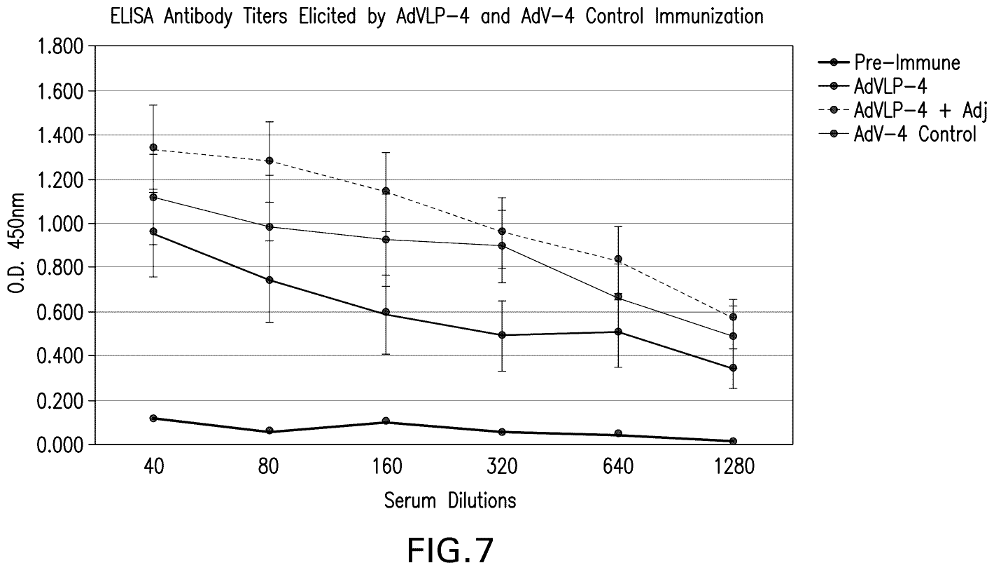

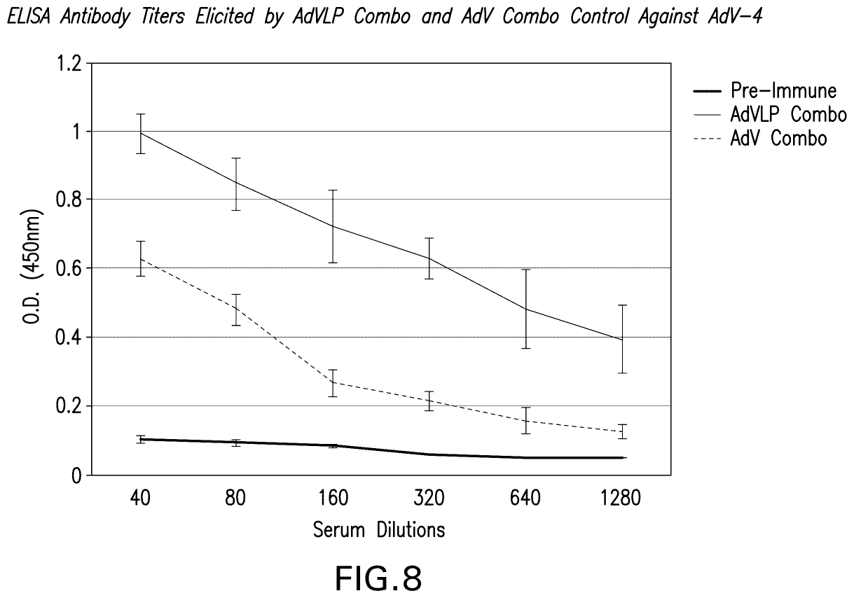

The immune response elicited in small animal models was tested following the administration of monovalent AdVLP as vaccine compositions (e.g., containing AdVLP4, AdVLP7, AdVLP14 or AdVLP55), as well as polyvalent formulations combining different monovalent AdVLPs, (e.g., a trivalent composition of AdVLP4, AdVLP7 and AdVLP14) or a tetravalent composition including of AdVLP4, AdVLP7, AdVLP14 and AdVLP55. These studies demonstrate that the monovalent vaccine compositions stimulated the production of high titers of specific serum total IgG antibodies as measured by ELISA. Either, AdVLP vaccine alone or when formulated with an adjuvant, stimulated production of significant levels of specific IgG as compared to the pre-immune control. A representative example of an ELISA using adenovirus 4 (AdV4) as antigen to evaluate the immune response elicited by the monovalent AdVLP4 is shown in FIG. 7. This shows that the AdVLP4 vaccine elicits a strong IgG specific response against AdV4, which is slightly higher than that elicited by an adenovirus 4 (AdV-4) immunized control when formulated with Alum and moderately lower than when formulated alone. Similarly, the trivalent formulation (Combo) elicited high IgG titers against AdV4, which are also slightly above those stimulated by the AdV virus combo control, FIG. 8. This study indicated that the AdVLP vaccines result in a robust serum IgG response, as represented by the comparison with those raised against AdV4. Analogous responses were elicited by the other monovalent compositions or components of the polyvalent formulations.

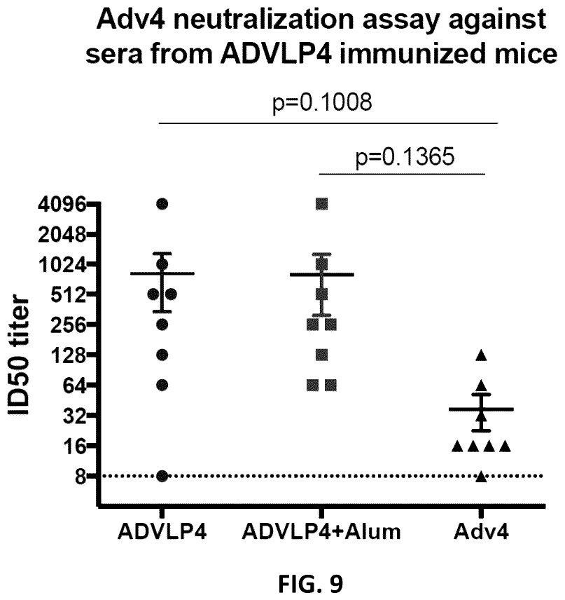

Furthermore, the specific neutralizing activity of the serum samples obtained from animals immunized with the monovalent and polyvalent AdVLP vaccine compositions were tested utilizing recombinant reporter adenovirus (rrAdV) which, following cell infection, express the reporter proteins luciferase and green fluorescent proteins (Luc/GFP). Detection of the levels of expression of these reporter genes is used to assess the power of the serum to prevent/neutralize viral infection. Interestingly, these vaccine compositions elicited high titers of neutralizing antibodies as compared to the response elicited by homologous inactivated adenovirus vaccine controls. An example of the neutralizing activity elicited against rrAdV4/Luc virus detected in serum samples from mice immunized with the monovalent AdVLP4 alone or admixed with an adjuvant as well as the inactivated adenovirus 4 (AdV4) control is shown in FIG. 9.

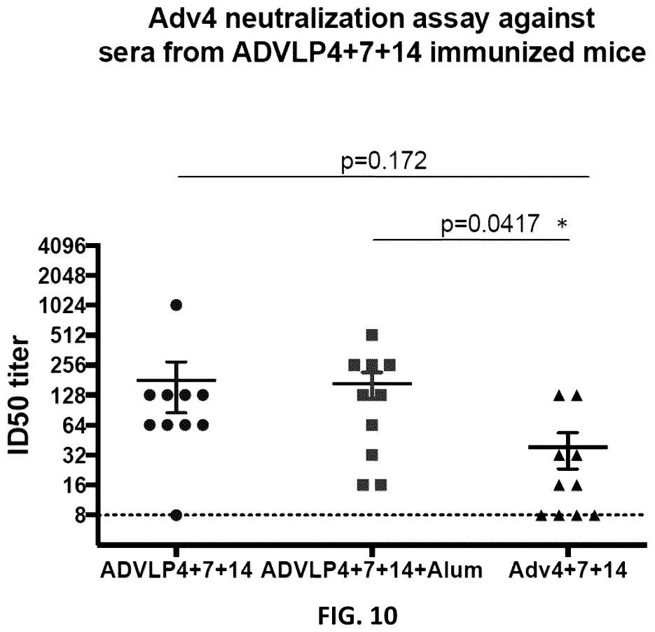

Both monovalent AdVLP4 vaccine formulations elicited strong neutralization activity with an average titer of about −750 ID50, which were greater than that elicited by the inactivated AdV4 virus immunized control. This difference, however, was not statistically significant. These results demonstrate that the AdVLP vaccine formulated either adjuvant or with buffer alone is able to elicit a potent neutralizing antibody response, FIG. 9. Furthermore, the neutralization activity against adenovirus 4 (rAdV4/Luc) detected in the serum samples from mice immunized with the combination AdVLP 4,7,14 vaccine alone or admixed with adjuvant (Alum) was also strong and higher than the one elicited by the inactivated adenovirus 4, 7, and 14 combination used as control, FIG. 10. In this case, the polyvalent combination of AdVLP4,7,14 vaccine admixed with alum induced neutralizing titers that were statistically significantly higher than the one elicited by the inactivated adenovirus 4,7,14 combination vaccine control. Thus, the trivalent AdVLP4,7,14 combination elicited a robust neutralizing antibody response that was superior to the response induced by the inactivated adenovirus 4, 7 and 14 combo vaccine control. Thus, both the monovalent and trivalent vaccine compositions elicited high titers of neutralizing response able to block infection of the homologous AdV4 virus.

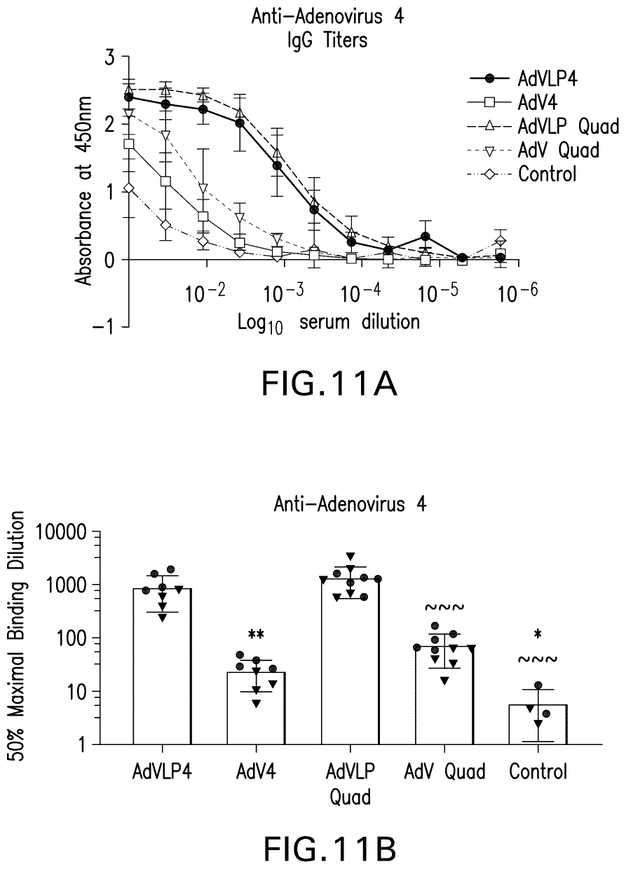

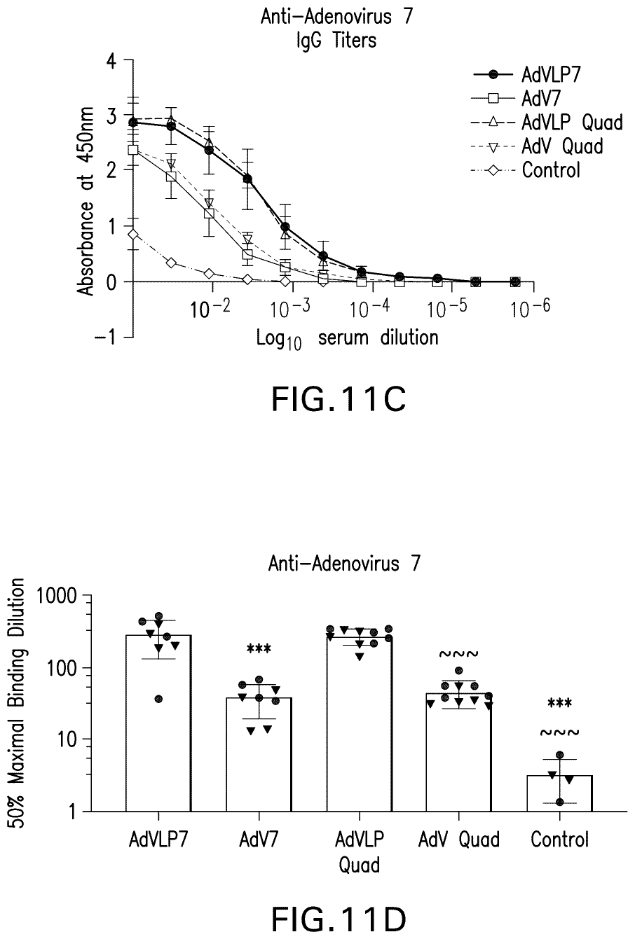

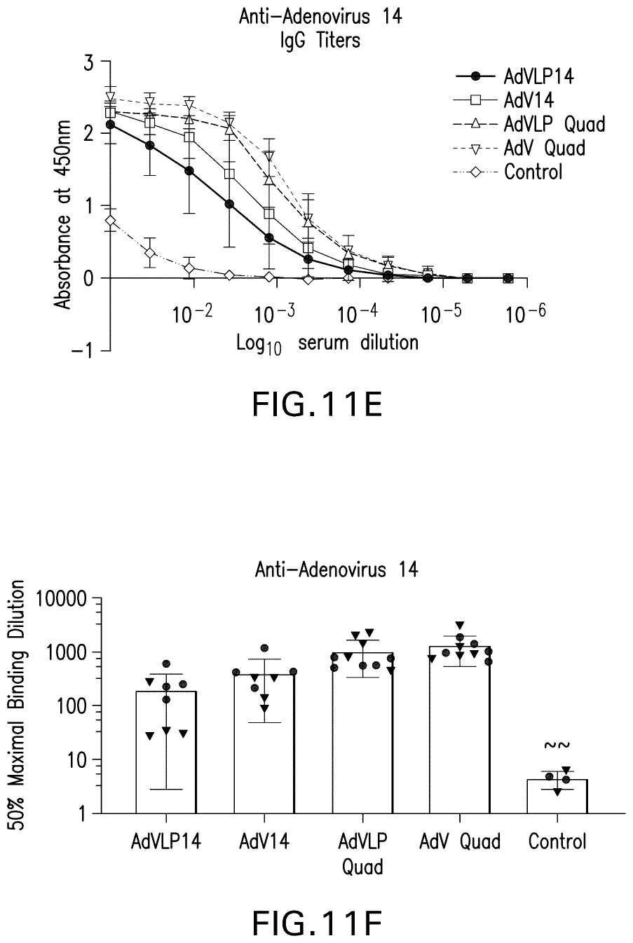

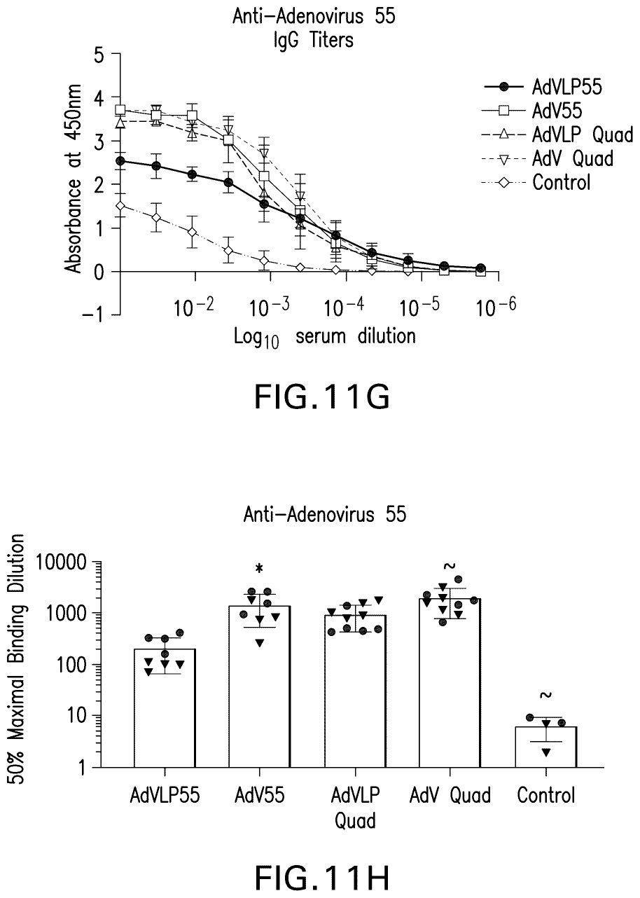

The other monovalent compositions AdVLP7, AdVLP14 and AdVLP55 as well as a quadrivalent formulation that included AdVLP4, 7, 14 and 55 were also able to stimulate strong and specific serum IgG responses as well as robust neutralizing antibody responses as compared to naïve controls or live adenovirus vaccinated controls. An example of the IgG responses elicited by monovalent vaccines and a quadrivalent formulation is shown in FIGS. 11A-11H.

In summary, the adenovirus-like particles (AdVLPs) assembled and produced using the methods described above stimulate robust and specific immune responses when administered as monovalent or polyvalent vaccine compositions. These vaccines can be delivered as a liquid formulation via the parenteral route; however, alternative formulation, (e.g., powder, gels, pills, etc.), and ways of delivery (e.g., epidermal, oral, intranasal, etc.) can also be utilized.

### 3. AdVLP7 Examples

In the following examples, in accordance with one or more embodiments, a set of proteins are defined that are used for the self-assembly of AdV-7 VLPs (AdVLP-7) in a mammalian expression system, and it is shown that these AdVLPs are comparable to wild type AdV-7 virus particles. These examples demonstrate that recombinant AdV capsid assembly can be driven by the expression of plasmid-encoded structural proteins. Immunogenicity studies were performed in mice, revealing that AdVLP-7 induces a potent humoral response against AdV-7.

### Materials and Methods

### Cells and Culture Conditions

HEK-293 cells (Gibco, Waltham, MA, USA) were grown in suspension culture in EX-CELL® CD HEK293 Viral Vector medium (Sigma Aldrich, St. Louis, MO, USA, 14385C) supplemented with 5 mM L-glutamine. A549 cells (ATCC, Manassas, VA, USA, CCL-185) were grown as adherent monolayers in Ham's F-12K (Kaighn's) medium (Gibco, 21127022) supplemented with 10% heat-inactivated FBS and 1× penicillin/streptomycin (100 U penicillin and 100 μg streptomycin per mL). All cultures were incubated in a humidified incubator at 37° C. with 5% CO₂. HEK-293 cells were incubated while shaking at 125 rpm.

### Viruses

Wild type AdV-7 was obtained from ATCC (strain Gomen, ATCC, VR-7). A replication-competent reporter AdV-7 (rAdV-7) with a deleted E3 region replaced with GFP, generated as previously described⁷⁰, was used for neutralization assays. Prior to use, wild type AdV-7 was passaged in HEK-293 cells, and rAdV-7 was passaged in A549 cells.

### Plasmids

The genes encoding the major capsid proteins (hexon, penton, and fiber), minor capsid/cement proteins (VIII, VI, IX, and IIIa), and accessory proteins L4-100k and L1-52/55k were codon optimized, chemically synthesized, and individually cloned into cloning vectors by Blue Heron Biotech (Bothell, WA, USA). Genes were subcloned from cloning vectors into the expression plasmid pcDNA3.4 by restriction enzyme digestion and ligation. Genes were consolidated into four plasmids, as follows: i. pcDNA3.4-hexon-IRES-100k (pHexon-100k), ii. pcDNA3.4-penton-IRES-fiber (pPenton-Fiber), iii. pcDNA3.4-CMV-VIII-IRES-VI-CMV-IX-IRES-IIIa (pVIII-VI-IX-IIIa), and iv. pcDNA3.4-52/55k (p52/55k). Constructs were verified by restriction enzyme digestion and sequencing.

### Production of AdVLPs

AdVLPs were produced by transfecting HEK-293 cells with the four plasmids listed above (i. pHexon-100k, ii. pPenton-Fiber, iii. pVIII-VI-IX-IIIa, and iv. p52/55k, in a 2:1:1:1 ratio). Cells were seeded at 1.0×10⁶ cells/mL one day prior to transfection. Transfection was conducted using PEI Max® (Polysciences, Warrington, PA, USA, 24765). PEI Max® was mixed with DNA in a 4:1 ratio (PEI:total DNA) in a volume of EX-CELL® CD HEK293 Viral Vector medium equal to 5% of the total culture volume. Mixtures were incubated for 15 minutes at room temperature to allow PEI/DNA complexation, and subsequently added to cells dropwise. Valproic acid was added to cells 24 hours after transfection at a final concentration of 3.75 mM. At 72 hours post transfection, transfected cultures containing intracellular AdVLPs were supplemented with 50 mM NaCl, 1 mM MgCl₂, and 1× Halt™ protease and phosphatase inhibitors (Thermo Scientific, Waltham, MA, USA, 78440).

### Purification of AdVs and AdVLPs

Cells containing AdVs or AdVLPs were subjected to three freeze-thaw cycles to lyse cells. Cell lysates were centrifuged at 10,000×g for 20 minutes at 4° C., and pellets of cellular debris were discarded. For AdVLPs, supernatants were concentrated 10× by tangential flow filtration using a Pellicon® XL50 with Biomax® 300 kDa membrane (Millipore Sigma, Burlington, MA, USA, PXB300C50). For both AdVs and AdVLPs, supernatants were loaded onto a two-step cesium chloride (CsCl) gradient (1.41 g/mL and 1.26 g/mL) in ultracentrifuge tubes and separated by ultracentrifugation using an SW28 rotor for two hours at 10° C. AdV samples are separated into two distinct bands, the lower (heavier) of which contains mature, infectious particles with packaged genomic DNA (referred to as WT AdV-7), while the higher (lighter) band consist of empty capsids which do not contain DNA (referred to as empty capsids). AdVLP samples contain only a single band. Each tube was punctured using an 18-gauge needle and separate fractions were collected for each band of interest. Fractions were diluted with 1.3 g/mL CsCl to a total volume of 11.5 mL. Samples were ultracentrifuged using an SW40ti rotor for 16 hours at 10° C. Bands of interest were again collected by puncturing the sidewall of the tube and fractions were stored in CsCl with 2 mM MgCl₂ at 4° C. until just prior to use. Immediately before use, samples were buffer exchanged using Amicon® Ultra centrifugal filter units with 100 kDa NMWCO (Millipore Sigma, UFC9100) into VLP suspension buffer (PBS with 187 mM NaCl, 2 mM MgCl₂, 6 μM Tween-80, and 0.1 mM EDTA).

### Protein Composition Analysis by Coomassie Staining, Western Blot, and Densitometry

Protein composition of purified AdVLP-7 and AdV-7 preparations was assessed using SDS-PAGE. Purified samples were mixed with lithium dodecyl sulfate sample buffer with β-mercaptoethanol and incubated at 85° C. for 5 minutes. Samples were separated by SDS-PAGE using 4-12% Bis-Tris gels (Invitrogen, Waltham, MA, USA). For visualization of total protein, gels were stained with Coomassie brilliant blue R-250 for 24 hours and de-stained with destaining solution (40% methanol, 10% glacial acetic acid) for 1 hour (destaining solution replaced with fresh solution every 15 minutes). For western blots, separated proteins were transferred to nitrocellulose membranes. Membranes were then blocked with 5% non-fat dry milk in TBS-T (20 mM Tris, 150 mM NaCl, 0.1% Tween-20). Membranes were incubated overnight with one of the following antibodies, as indicated: i. goat anti-adenovirus 5 antibody (1:1,000 dilution, Novus Biologicals, Centennial, CO, USA, NB600-1386), ii. anti-IIIa, iii. anti-AdV-14, iv. anti-VII, v. anti-IX, or vi. anti-L1-52/55k (ii.-vi. used at 1:200 dilution, generated in house via immunization of rabbits with the respective His-tag purified proteins). Membranes were washed 3× with TBS-T for 10 minutes. HRP-conjugated rabbit anti-goat IgG (1:6,000 dilution, Abcam, Cambridge, UK, ab97100) or goat anti-rabbit IgG (1:6,000 dilution, Invitrogen, 65-6120) was added to membranes and incubated for 1 hour. Membranes were again washed 3× with TBS-T for 10 minutes and subsequently developed using the SuperSignal West Pico PLUS chemiluminescent substrate (Thermo Scientific, 34580). Coomassie-stained gels and developed western blots were imaged using an Azure Biosystems C600 Imaging System. Protein quantification was performed via densitometry analysis of western blots using AzureSpot software (Azure Biosystems, Dublin, CA, USA). Serial dilutions of purified hexon protein were used as standards.

### Particle Size Analysis by Dynamic Light Scattering

Particle size of purified samples was determined by dynamic light scattering using a Litesizer 500 (Anton Paar, Graz, Austria). Purified samples were diluted 20× in PBS and loaded into a cuvette. Measurements were performed using default settings for protein samples in PBS. The hydrodynamic diameter of each sample was measured in series of at least 5 replicates.

### Negative Staining Electron Microscopy

CF200-CU carbon film 200 mesh copper grids (Electron Microscopy Sciences, Hartfield, PA, USA) were held with forceps and washed with 10 μL of 0.01% BSA solution. After a 5 second incubation, grids were dried using filter paper to draw liquid off from the edge. Samples of AdVLP-7 (5 μL) were immediately loaded onto grids and allowed to incubate at room temperature for 5 minutes. After incubation, grids were again dried with filter paper. Grids were then immediately stained with 2% phosphotungstic acid and incubated for 1 minute at room temperature. After incubation, grids were dried a final time with filter paper, and further air-dried overnight. Grids were examined using a JOEL 2100 transmission electron microscope at 200 kV and imaged with a 2048×2048-pixel CCD (Gatan Inc, Pleasanton, CA, USA).

### Immunization of Mice

Immunogenicity of AdVLP-7 vaccines was tested in 6-8 weeks old BALB/c mice purchased from Charles River Laboratories (Wilmington, MA, USA). AdVLP-7 vaccines were given to groups of mice, adjuvanted with either aluminum hydroxide (Alum) or AddaVax (InvivoGen, San Diego, CA, USA), with a third group receiving AdVLP-7 formulated without adjuvant. For control, a fourth group was administered wild type AdV-7 without adjuvant. Finally, a fifth group received only a sham injection with VLP suspension buffer. Mice were immunized with primary and booster doses of vaccines or sham, administered via intramuscular injection into a hind leg on days 0 and 14, respectively. Each dose of AdVLP-7 or AdV-7 contained 4 μg of hexon protein, determined by densitometry analysis of western blots. Serum samples were collected three weeks after the booster immunization (day 35). All sera were heat-inactivated by heating at 56° C. for 30 minutes and subsequently stored at −80° C.

### Assessment of Humoral Immune Response by ELISA

Antibody titers were determined for all serum samples via ELISA. Total IgG titers against each of the major capsid proteins were measured individually, in addition to titers against total virion. IgG subclass analysis was also performed against purified total virion. Purified major capsid proteins (hexon, penton, or fiber) or purified AdV-7 particles were diluted to a concentration of 0.5 μg/mL in coating buffer (0.1 M sodium bicarbonate, pH 9.6) and 100 μL was added to each well of a 96-well plate. Plates were incubated overnight at 4° C. Following overnight incubation, plates were blocked with 1% BSA in PBS for 1 hour at room temperature and subsequently washed with 0.05% Tween 20 in TBS. Serum samples (n=10 per group) were added to plates, serially diluted 3-fold in blocking buffer, and incubated for 2 hours at room temperature. Plates were then washed and incubated with one of the following HRP-conjugated secondary antibodies: for quantification of total IgG, goat anti-mouse IgG (1:10,000 dilution, IgG heavy and light chain, Invitrogen, 31430); for IgG subclass analysis, either goat anti-mouse IgG1 (1:4,000 dilution, IgG1 heavy chain, Southern Biotech, 1071-05) or goat anti-mouse IgG2a (1:4,000 dilution, IgG2a heavy chain, Southern Biotech, 1081-05). Secondary antibodies were diluted in blocking buffer and added to plates for 1 hour at room temperature. Plates were washed a final time and developed with TMB substrate (Thermo Scientific, 34029) for 15 minutes at room temperature. The reaction was stopped by adding 2 M sulfuric acid. The absorbance at 450 nm was determined for each well. For each serum sample, absorbance was plotted against the dilution factor. Binding titers, defined as the serum dilution at which absorbance readings were at 50% of their maximum value, were calculated using a sigmoidal 4-paramter logistic regression. Serum samples that showed binding titers that fell below the lower limit of detection were assigned a value equal to half of the starting serum dilution to enable calculation and statistical analyses.

### Microneutralization Assay

Neutralizing antibody titers in sera were assessed using a recombinant reporter AdV-based microneutralization assay. Heat-inactivated sera (n=10 per group) were 2.5-fold serially diluted in 96-well plates in Ham's F-12K (Kaighn's) medium with 5% FBS and 1× penicillin/streptomycin. Serum dilutions were mixed with an equal volume of GFP-expressing rAdV-7 containing ˜500 FFU, for a total mixture volume of 100 μL. Each serum sample was run in triplicate, with a starting dilution of 1/40 (indicative of the serum dilution after mixing with rAdV-7). Serum/virus mixtures were incubated for 1 hour at 37° C., at which point 2.25×10⁴ A549 cells in 100 μL F-12K were added to each well and mixed. The following controls were included: i. cells exposed only to the rAdV-7 (no serum); and ii. cells unexposed to either serum or rAdV-7 (background fluorescence control). Plates were incubated at 37° C. with 5% CO₂ for 28 hours. Following incubation, plates were imaged using a Celigo Image Cytometer (Nexcelom Bioscience, Lawrence, MA, USA). The number of green fluorescent cells in each well was measured and plotted against the serum dilution factor. Plots were fitted with a non-linear regression, which was used to calculate the serum dilution at which 50% of the rAdV-7 was neutralized relative to control wells that contained cells infected with rAdV-7 unexposed to serum (ID₅₀). Neutralizing antibody titers that fell below the lower limit of detection were assigned a value of 20, equal to half the starting serum dilution factor, to allow for statistical analysis.

### Statistical Analyses

For comparison of results of ELISA and microneutralization assays between groups, data were analyzed by one-way analysis of variance (ANOVA) with Tukey's multiple comparisons test. Correlation analyses were performed using Pearson's correlation analysis. For all analyses, alpha=0.05. All statistical analyses were performed using GraphPad Prism 9 software (GraphPad Software, San Diego, CA, USA).

### Results:

### Purification and Characterization of AdVLPs Produced in a Mammalian Expression System

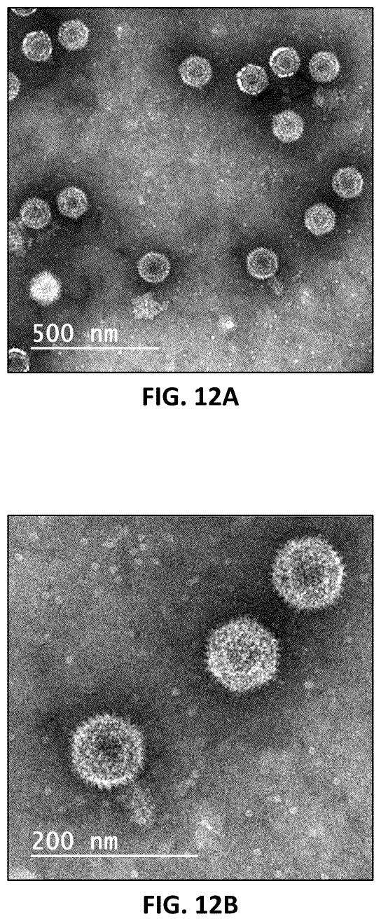

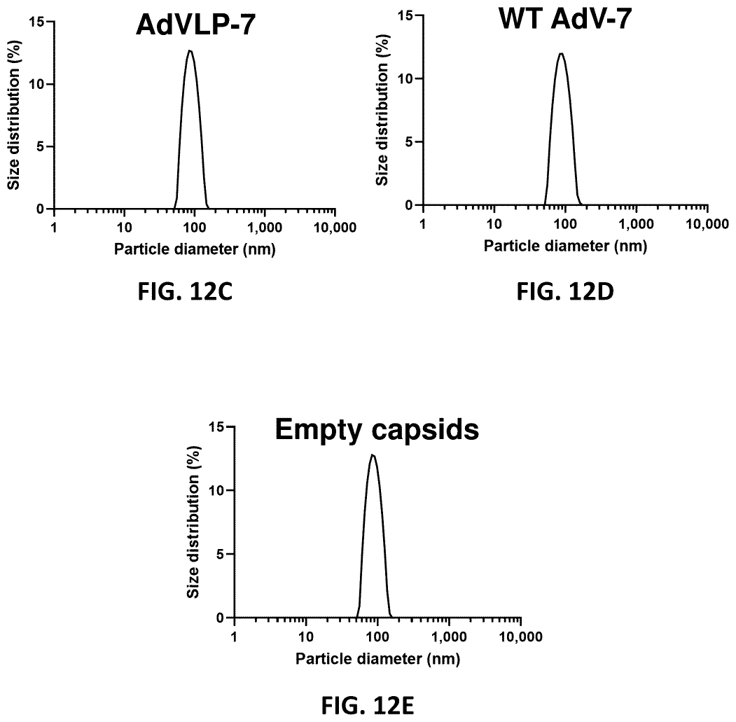

The minimum set of proteins needed for formation of stable AdVLP-7 particles was identified, which includes the major capsid proteins hexon, penton, and fiber, minor capsid/cement proteins IIIa, VI, VIII, and IX, the chaperone protein L4-100k, and the accessory scaffold protein L1-52/55k. To produce AdVLP-7, four expression plasmids encoding the required viral genes were introduced into HEK-293 cells by transient transfection. Particles were harvested by repeated freeze/thaw cycles of transfected cells, and purified by cesium chloride gradient ultracentrifugation. Purified particles were examined by negative staining electron microscopy, which revealed that the structure of AdVLP-7 mimics the typical icosahedral morphology of wild type AdVs (FIGS. 12A-12B). AdVLP-7 particles were also confirmed by dynamic light scattering (DLS) to be 80-90 nm in diameter (FIG. 12C). DLS profiles for AdVLP-7 were nearly identical to those observed for wild type AdV-7 capsids, both with (WT AdV-7, FIG. 12D) and without packaged genomic DNA (Empty capsids, FIG. 12E). Despite a lack of genetic material, purified AdVLP-7 particles maintained the same DLS profile as in FIG. 12C over 40+ weeks of storage at 4° C., indicative of long-term stability (data not shown).

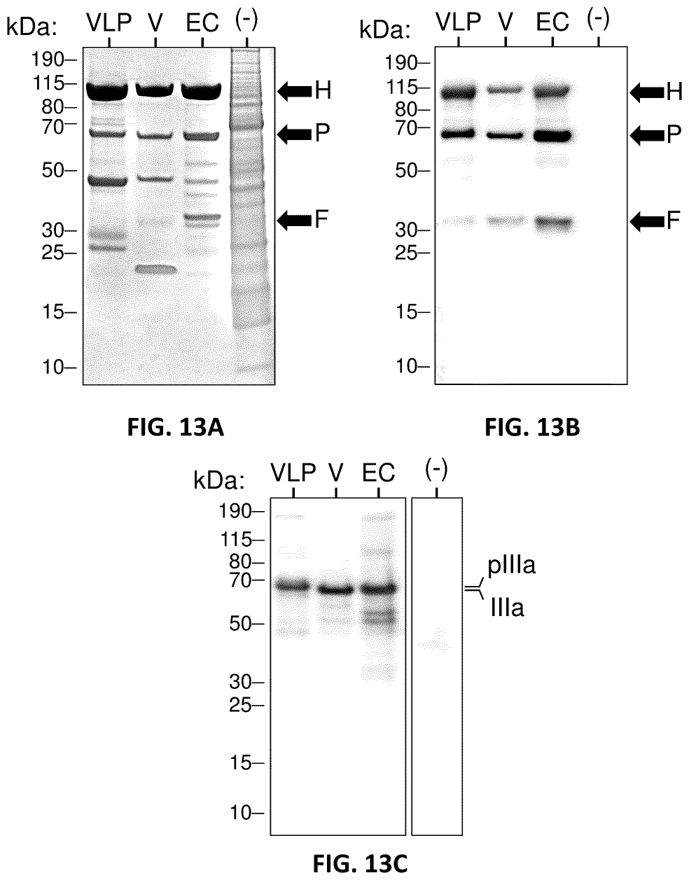

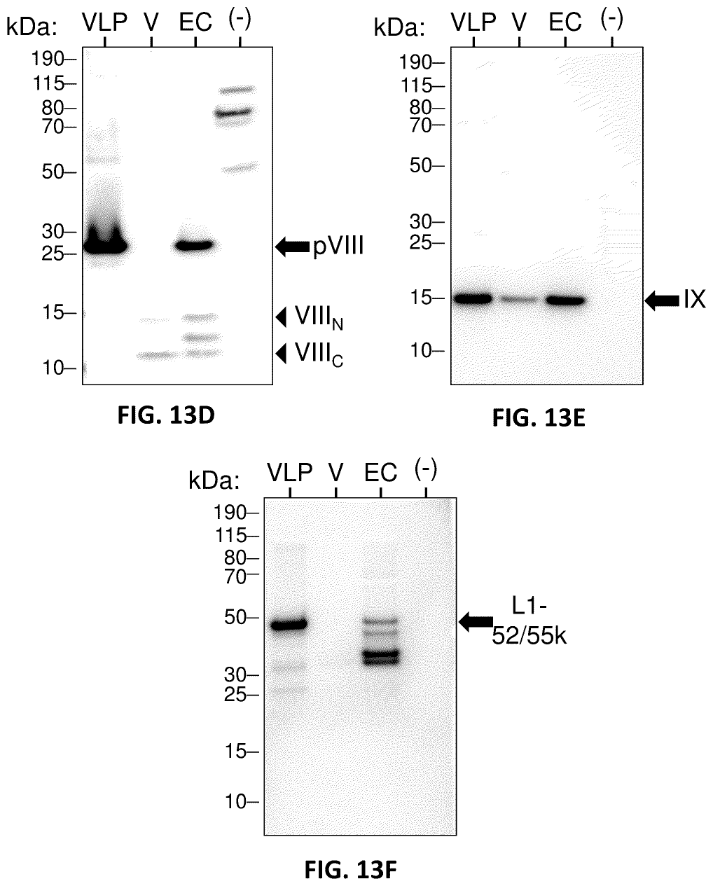

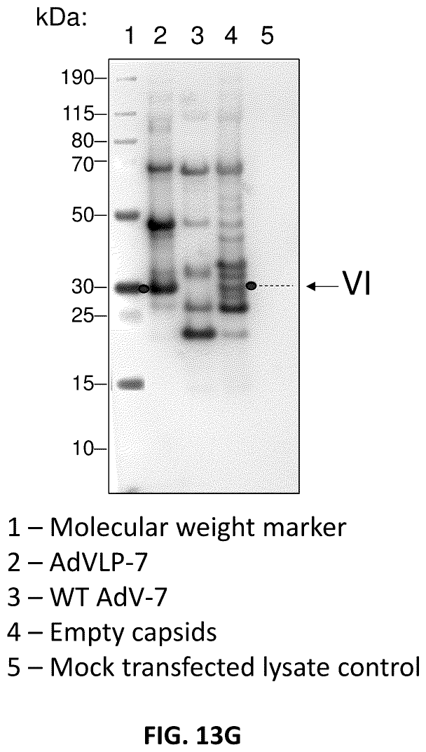

Protein compositional analysis of AdVLP-7 was performed by SDS-PAGE with subsequent Coomassie blue staining (FIG. 13A) or western blotting (FIG. 13B). Each of the major capsid proteins (hexon (105.2 kDa), penton (61.9 kDa), and fiber (35.3 kDa)) were detected by Coomassie blue staining in purified AdVLP-7 (FIG. 13A). These proteins were also detected in control samples of WT AdV-7 and empty capsids of AdV-7 (FIG. 2a). Gels stained with Coomassie blue demonstrate the high degree of purity of AdVLP-7 preparations. Western blots probed with an anti-AdV polyclonal antibody also showed the appropriate bands for each of the major capsid proteins in AdVLP-7, WT AdV-7, and empty capsids (FIG. 13B). Cell lysate of mock transfected HEK-293 cells did not indicate any non-specific binding of the antibody (FIG. 13B). In addition to the major capsid proteins, probing was conducted using antibodies against minor capsid proteins IIla (65.7 kDa, FIG. 13C), VIII (24.9 kDa, FIG. 13D), and IX (14.1 kDa, FIG. 13E), and accessory scaffold protein L1-52/55k (44.3 kDa, FIG. 13F), each of which were detected in the AdVLP-7 sample. Internally located minor capsid/accessory proteins (IIIa, VIII, and L1-52/55k) appear on blots as unprocessed precursor proteins in the AdVLP-7 sample. However, in the WT AdV-7 samples, IIIa and VIII appear as proteins that have been processed by the adenovirus protease (AVP), while L1-52/55k is expectedly absent from the mature capsids, as it is removed during the final maturation processes⁴⁴. The immature, morphologically incomplete empty capsids of AdV-7 showed signs of the intermediate stages of processing by the AVP; IIIa appears to be completely cleaved (FIG. 13C), while protein VIII and L1-52/55k are detected in both processed and unprocessed forms (FIGS. 13D and 13F). Protein IX, which is located on the external faces of the capsid, is not processed by the AVP, and appears as a 14.1 kDa band in AdVLP-7, WT AdV-7, and empty capsid samples (FIG. 13E). None of the antibodies against minor capsid proteins or L1-52/55k showed non-specific binding in the size range of interest in mock transfected lysates.

### AdVLP-7 Elicits High Titers of Antibodies Against AdV-7 in BALB/c Mice

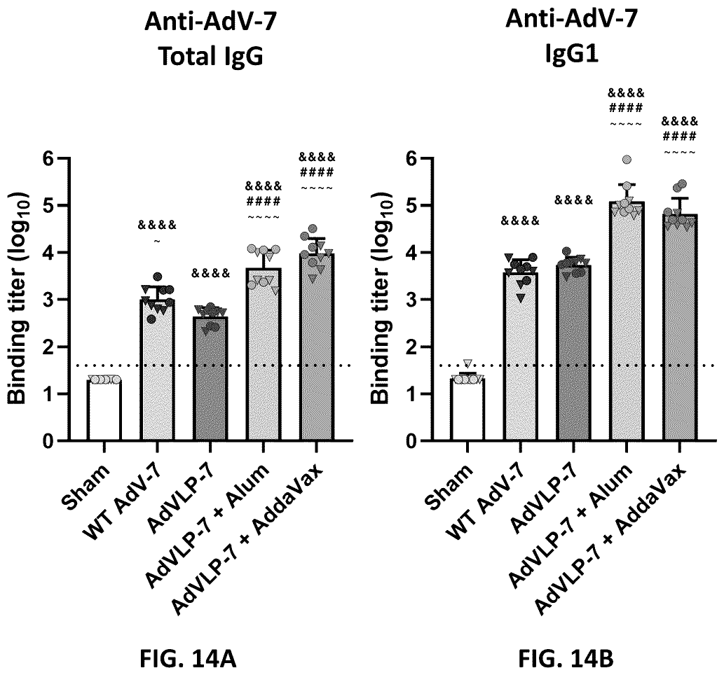

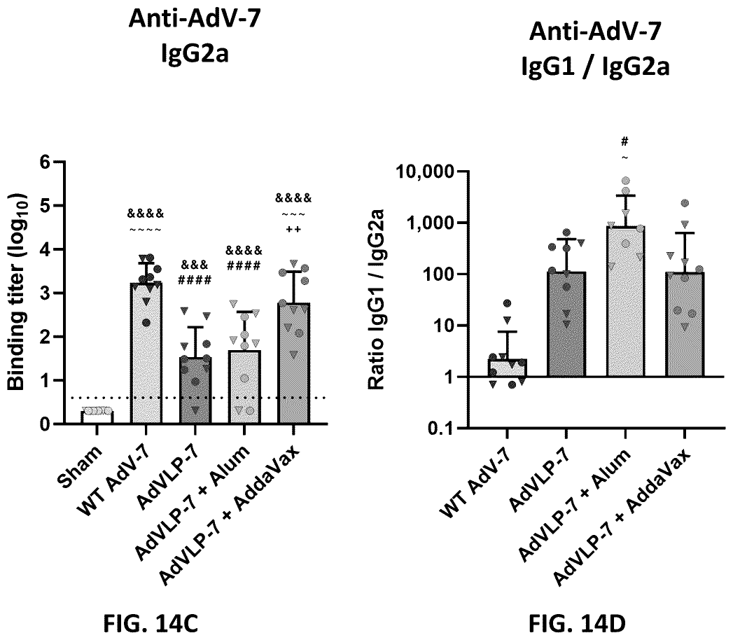

To determine the immunogenicity of these recombinant capsids, BALB/c mice were immunized with purified AdVLP-7, administered alone or in combination with an adjuvant (aluminum hydroxide or AddaVax™, a squalene-based oil-in-water nano-emulsion). To examine how AdVLP-7 compares to the wild type virus, a separate group of mice were immunized with WT AdV-7. As a control, an additional group of mice received only a sham injection of buffer. Mice were given the initial dose and then boosted two weeks later. Three weeks after the final immunization, serum samples were collected and AdV-7-specific antibody titers were measured via ELISA. All vaccinated mice generated higher titers of IgG antibodies against AdV-7 as compared to sham-injected mice, which did not show any binding activity against AdV-7 (FIG. 14A). Immunization with AdVLP-7 alone resulted in anti-AdV-7 IgG titers similar to those seen in mice immunized with WT AdV-7. Furthermore, mice that received AdVLP-7 adjuvanted with either alum or AddaVax had significantly higher anti-AdV-7 IgG titers when compared with those immunized with AdVLP-7 alone or WT AdV-7 (FIG. 14A). Within each group, significant differences in anti-AdV-7 IgG titers between males and females were not observed.

To better understand the effects of vaccination with AdVLP-7 on the cellular immune response, the titers of both IgG1 and IgG2a were determined, as these are markers of Th2- and Th1-skewed responses, respectively. AdVLP-7-immunized mice generated high titers of both IgG1 and IgG2a against AdV-7, regardless of adjuvant formulation (FIGS. 14B-14C). Specifically, AdVLP-7 adjuvanted with alum demonstrated a high IgG1/IgG2a ratio, indicative of a strong Th2 response, whereas mice vaccinated with either AdVLP-7 alone or AdVLP-7 adjuvanted with AddaVax demonstrated a more balanced response (FIG. 14D). In contrast, mice vaccinated with WT AdV-7 generated a higher IgG2a response than AdVLP-7-immunized mice, highlighting the strong Th1 response associated with intracellular infections (FIG. 14D).

### Antigen-Specific Humoral Response

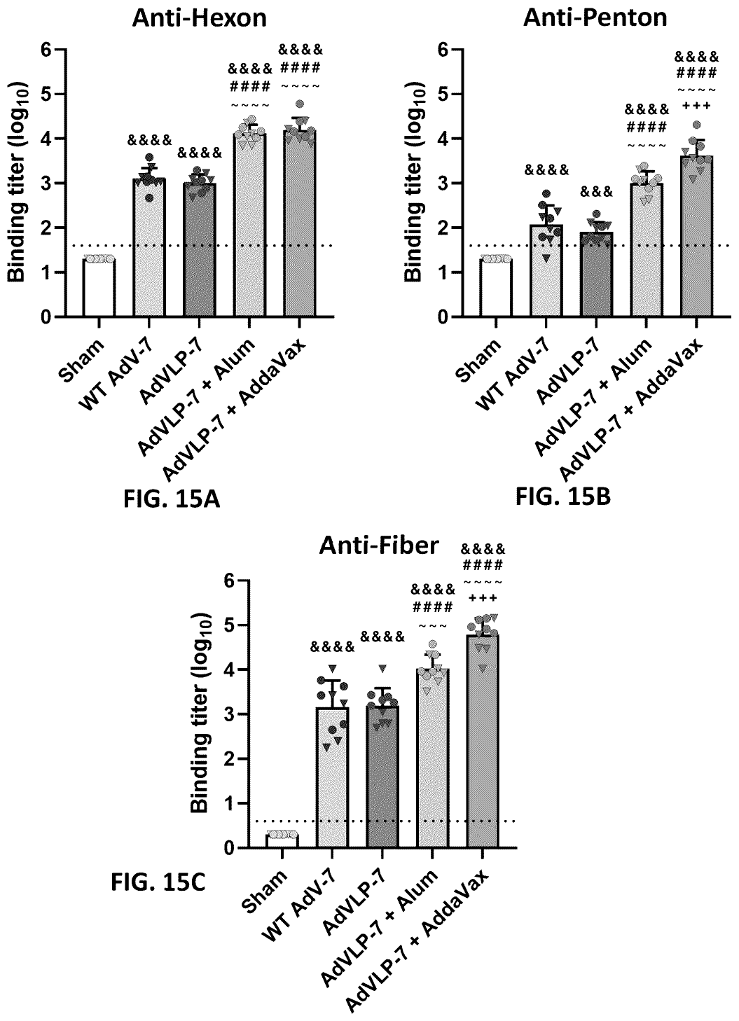

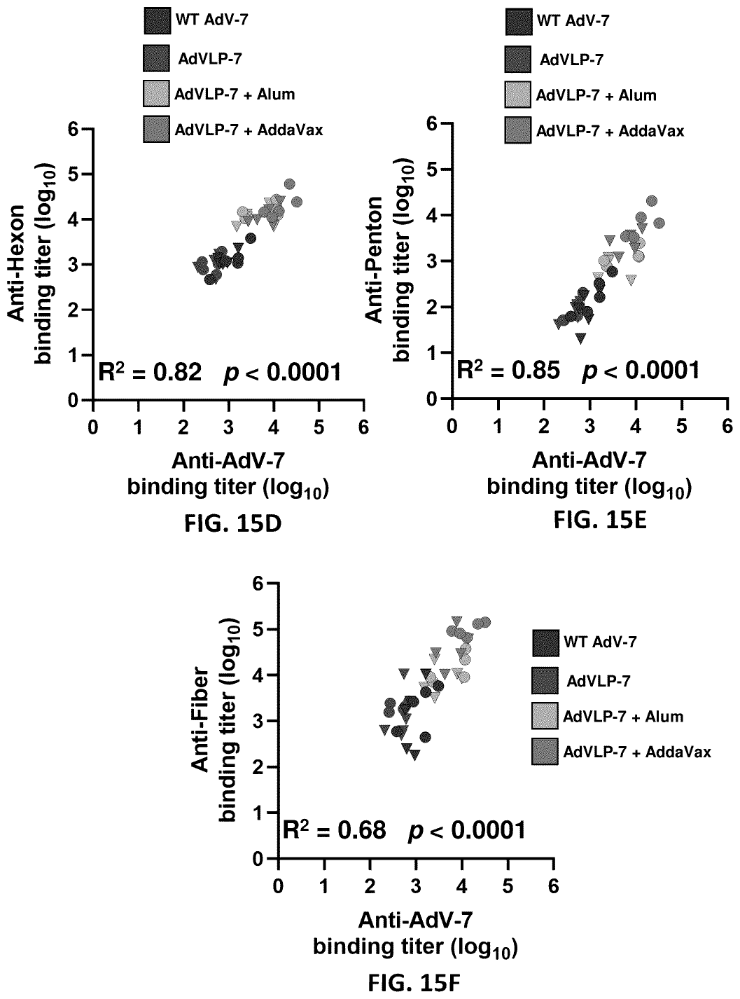

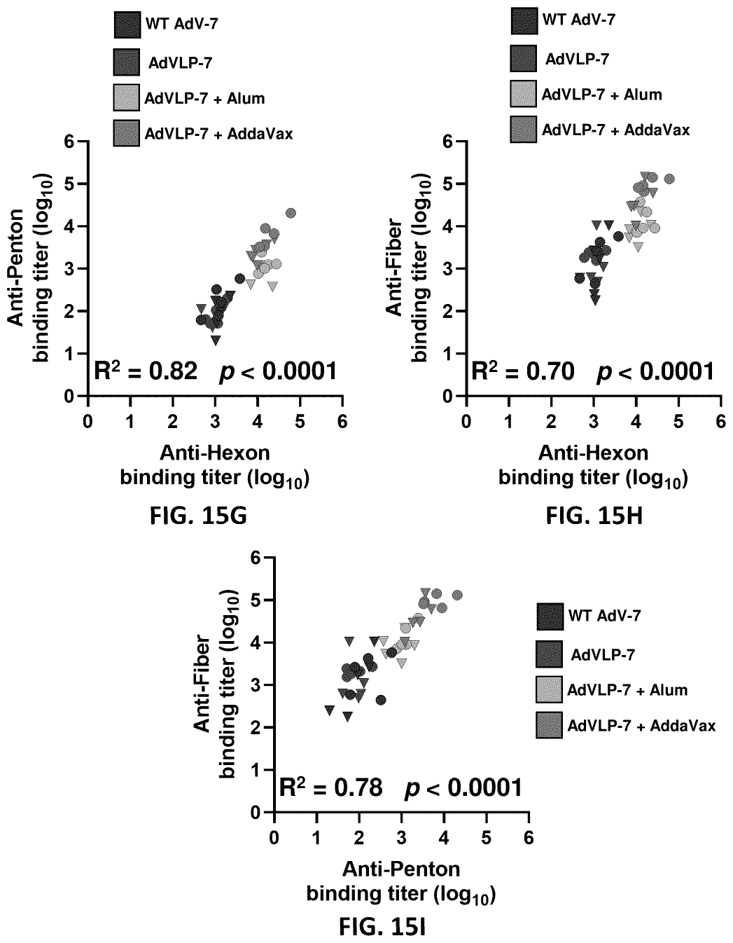

While the cellular immune response is critical to the resolution of AdV infection^{45,46}, the hallmark of protection against infection is neutralizing antibodies (NAbs). Major capsid proteins hexon, penton, and fiber are the predominant targets of NAbs generated during AdV infection³⁸⁻⁴¹. The IgG titers against each major capsid protein were determined for all animals via ELISA. Immunization with AdVLP-7, alone or adjuvanted, resulted in significant IgG titers against all major capsid proteins (FIGS. 15A-15C). Mice vaccinated with WT AdV-7 generated IgG titers against hexon, penton, and fiber at the same level as mice immunized with AdVLP-7 alone (FIGS. 15A-15C), highlighting the ability of the AdVLP-7 to mimic the native viral capsid. When compared with the WT AdV-7 and AdVLP-7 alone groups, immunization with AdVLP-7 adjuvanted with either alum or AddaVax elicited significantly higher titers of IgG against each of the major capsid proteins (FIGS. 15A-15C). While the alum and AddaVax formulations elicited similar IgG titers against hexon (FIG. 15A), AdVLP-7 adjuvanted with AddaVax induced significantly higher IgG titers against penton and fiber as compared to AdVLP-7 adjuvanted with alum (FIGS. 15B-15C). Importantly, it was found that binding titers against each of the major capsid proteins correlated with binding titers against total AdV-7 (FIGS. 15D-15F). Similarly, titers against each individual major capsid protein correlated with those against all other major capsid proteins (FIGS. 15G-15I).

### AdVLP-7-Induced Antibodies Potently Neutralize AdV-7

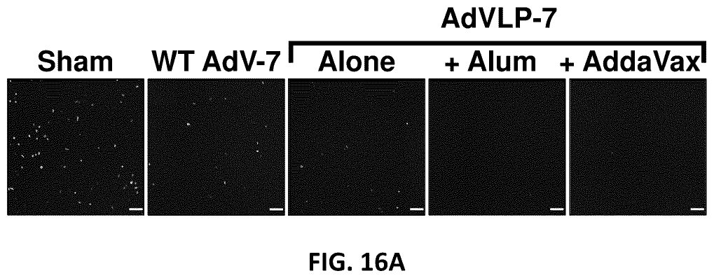

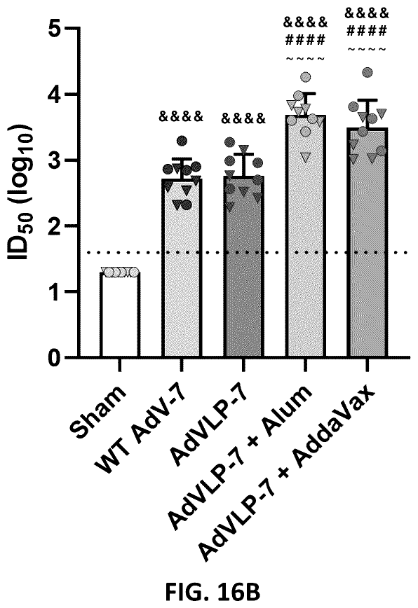

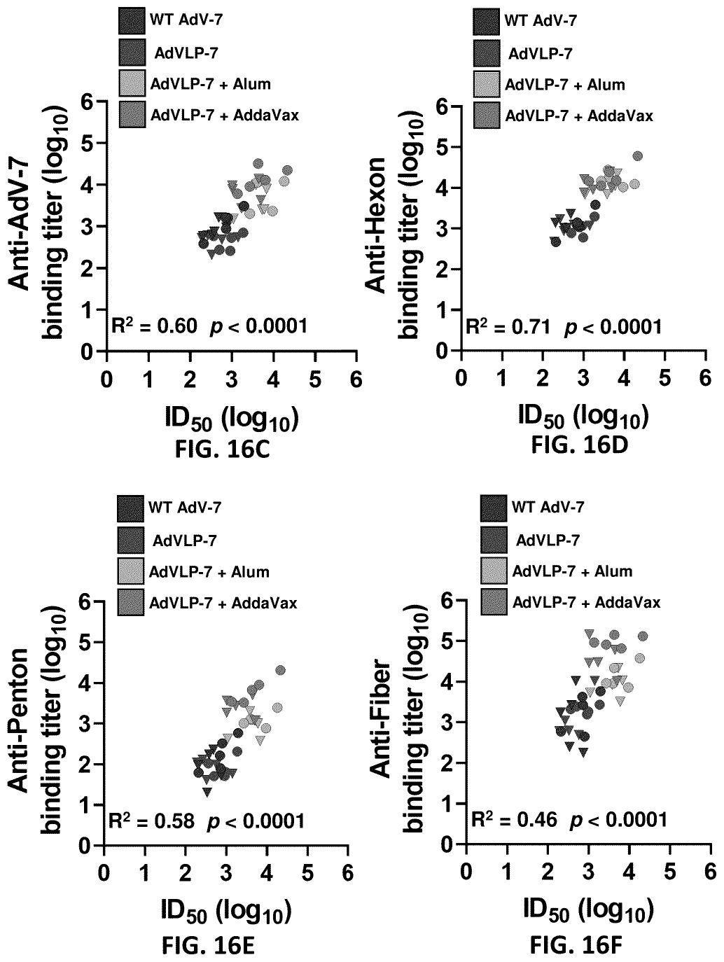

Given the high binding titers observed against the major capsid proteins, the functionality of the antibody response against AdV-7 in immunized mice was also assessed. Neutralizing activity was determined using a microneutralization assay based on a reporter AdV-7 that expresses GFP (rAdV-7). Incubation of rAdV-7 with serum from vaccinated mice led to a reduction in the number of infected cells as compared to the sham-injected group, which did not show neutralizing activity (representative images, FIG. 16A). NAb titers of the vaccinated groups were quantified, presented here as the serum dilution at which 50% of the signal from rAdV-7 was neutralized relative to control wells (ID₅₀, FIG. 16B). Interestingly, similar levels of NAbs were observed between the groups that received WT AdV-7 and AdVLP-7 alone (FIG. 16B). When formulated with alum or AddaVax, the AdVLP-7 vaccine elicited significantly higher titers of NAbs as compared to the groups immunized with WT AdV-7 or AdVLP-7 alone. While there is a clear beneficial effect of administering AdVLP-7 with an adjuvant, both of the tested adjuvants elicited similar titers of NAbs. At the individual animal level, a direct correlation between NAb titers and binding antibody titers against total AdV-7 virions (FIG. 16C) and individual major capsid proteins (FIG. 16D-16F) was observed. While all correlations were statistically significant (p<0.0001), NAb titers unsurprisingly correlated most strongly with binding antibody titers against hexon (R²=0.71, FIG. 16D).

As exemplified by the above Examples, development of an alternative to the live virus vaccines against Adenoviruses, such as AdV-4 and AdV-7, is critical for combating adenoviral infection not only in military populations but also in the general public. The existing live virus vaccines against AdV-4 and AdV-7 have proven to be very effective with >93% seroconversion rate in vaccinated individuals, and a vaccine efficacy of 99% against infection²⁴. However, live AdVs have the ability to undergo recombination which can produce new subtypes^{26,27}, and therefore pose a significant safety hazard. Considering the potential for recombination and vaccine-associated viral shedding that can persist for nearly a month after vaccination, the existing vaccines cannot be safely administered to the general public. While AdV infections are typically mild, more severe infections can occur, especially in susceptible populations including children, the immunosuppressed, and during pregnancy^{17,47-51}. The clinical manifestations of AdV infections are broad, but can include gastroenteritis, hepatitis, and pneumonia among many others, and can be fatal in both immunocompromised and immunocompetent patients^{3,11,52}. Given the significant risks and limitations associated with the current live virus vaccines, and considerable threat posed by AdV infections, better approaches to vaccination need to be developed.

One such approach is through the use of the virus-like particle platform of the present application. AdVLPs are structural mimics of native viruses, enabling antigens to be presented to the immune system in the same conformation as the live virus vaccines. However, AdVLPs are non-replicating as they lack genomic material, and therefore present no risk of recombination or vaccine-associated shedding. While VLP-based vaccines have been shown to be safe and effective in humans, only a few have been successfully developed and brought to market³⁰⁻³⁴. The challenge in developing VLP vaccines is determining the correct conditions and composition that enable the formation of stable particles. As exemplified in the examples, the necessary components for the generation of AdVLPs are defined, and demonstrate the first construction of a recombinant capsid that mimics both the size and icosahedral structure of wild type AdVs.

For example, it was determined that formation of stable AdVLPs requires the expression of the major and minor capsid proteins, the chaperone protein L4-100k which is required for proper folding of hexon, and the accessory protein L1-52/55k. While L1-52/55k is primarily involved in genome packaging⁵³ and is not present in mature WT AdV-7 virions⁴⁴, for instance, it was found that AdVLPs that lack this protein are not stable for long durations. Indeed, AdVLPs formed without the minor/cementing proteins and the accessory protein L1-52/55k become unstable and are unsuitable.

Not included in AdVLP-7, for example, was the adenovirus protease (AVP), which is important for the final proteolytic processing of several proteins within assembled capsids, some of which are components of AdVLP-7 (IIIa, VI, VIII, and L1-52/55k)⁵⁴⁻⁵⁷. Cleavage of these proteins primarily functions in rendering the virus infectious, as it allows capsid uncoating to occur upon cell entry^{58,59}.

Additionally, AVP requires multiple cofactors, including viral DNA⁶⁰⁻⁶², for proper activation and therefore would not function correctly in VLPs which lack genomic material and are non-infectious in nature. The lack of AVP was apparent in western blot analyses, which showed that IIIa, VI, VIII, and L1-52/55k exist in their uncleaved conformations in AdVLP-7, but are processed or absent in the case of L1-52/55k in mature WT AdV-7, as expected. Differences in banding patterns between AdVLP-7 and WT AdV-7 are consistent with previous studies regarding precursor proteins and the AVP^{55,56,63-65}. Even without AVP, AdVLP-7 is stable for more than 40 weeks when stored at refrigeration temperatures.

In addition to defining the components needed for generation of stable AdVLPs, it was also found that these recombinant capsids are highly immunogenic. As exemplified in the above examples, AdVLP-7 elicited a robust humoral response in mice, resulting in high titers of antibodies that bind to each of the major capsid proteins and potently neutralize AdV-7. Importantly, the observed response was equivalent between the males and females of each individual group. AdVLP-7 not only mimics the size and structure of WT AdV-7, the recombinant capsids also induce an equal antibody response. When administered without an adjuvant, AdVLP-7 elicited nearly identical binding and neutralizing antibody titers to those observed in mice immunized with an equivalent dose of WT AdV-7.

Additionally, the immunogenicity of AdVLP-7 was significantly increased when adjuvanted with either alum or AddaVax, though both adjuvants elicited similar levels of total IgG and NAbs. This trend between the different vaccine groups was observed for all measures of total IgG titers (against hexon, penton, fiber, and total AdV-7), as well as NAb titers. Furthermore, there were significant correlations between all datasets, most notably between the total IgG and NAb titers. In general, mice that showed higher binding titers against AdV-7 and its major capsid proteins also tended to show higher neutralizing activity. Binding titers against hexon, the primary target of neutralizing antibodies³⁸⁻⁴⁰, expectedly correlated most strongly with NAb titers. These results highlight the overall consistency of the AdVLP vaccine strategy and the striking similarity between recombinant and wild type AdV capsids.

Neutralizing antibody titers are used as the benchmark for protection against AdV infection after vaccination^{22,24}, with less of an emphasis placed on the cellular immune response. However, the development of a strong T cell response is critical for controlling AdV infections⁶⁶. Both clinical and in vitro experiments have highlighted the role of AdV-specific T cells in immunity against infection and prevention of severe disease⁶⁷. In stem cell transplant recipients, the reconstitution of hexon-specific CD4⁺ and CD8⁺ T cells resulted in spontaneous resolution of disseminated AdV infection⁴⁵. To elucidate the effects of AdVLP-7 immunization on the T cell response, IgG1 and IgG2a titers were determined, as they are markers of the Th2 and Th1 response, respectively. The results showed that immunization with AdVLP-7 adjuvanted with alum results in a Th2-skewed response, as indicated by the high levels of IgG1 production and relatively low IgG2a titers. A more balanced response was induced when AdVLP-7 was administered either alone or adjuvanted with AddaVax. The administration of AdVLP-7 with AddaVax resulted in significantly higher titers of IgG2a antibodies than other AdVLP-7 formulations, comparable to those generated in mice immunized with WT AdV-7. However, the ratio of IgG1 to IgG2a was much lower in the WT AdV-7 group, indicative of a Th1 polarization. While this IgG subclass analysis presents a preliminary assessment, additional studies are needed to further understand the cellular response elicited by AdVLP-based vaccines.

Overall, these examples highlight the ability of AdVLPs to serve as a platform for the next generation of vaccines against human AdVs. There are several directions that can be explored to continue the development of the AdVLP platform. As shown and described in the above examples and throughout the present application, in accordance with one or more embodiments herein, a template for AdVLP generation has been developed.

Exemplary amino acid and nucleotide sequences of various proteins of the AdVLPs of the present application are shown below in accordance with or more embodiments.

Sequence Name: Hexon
Length: 2814
Type: DNA
Organism: Adenovirus 7
Other information:
Description of sequence: Synthetic
SEQ ID NO: 1
ATGGCCACCC CCTCTATGAT GCCACAATGG GCTTATATGC
ATATTGCCGG TCAAGACGCG TCTGAATACC TGTCACCCGG
ACTTGTGCAA TTCGCAAGAG CCACGGATAC CTATTTTAGT
ATGGGCAACA AGTTTCGGAA CCCTACAGTA GCTCCCACGC
ACGACGTTAC TACAGATAGA TCCCAACGGC TGATGCTGCG
GTTCGTTCCT GTGGACCGCG AAGATAACAC TTATAGTTAT
AAAGTGAGAT ACACCCTTGC TGTAGGAGAC AATCGAGTGC
TGGACATGGC TTCAACCTTT TTCGATATAC GGGGAGTATT
GGATCGCGGC CCTTCTTTCA AACCATATTC CGGCACAGCC
TATAATAGCC TCGCACCCAA AGGTGCCCCT AATACAAGTC
AGTGGATTGT AACCGCAGGA GAGGAACGCG CCGTGACTAC
AACAACAAAT ACATTTGGAA TTGCATCTAT GAAAGGGGAT
AATATAACCA AAGAGGGCCT GGAAATCGGT AAAGACATTA
CTGCCGATAA CAAGCCCATT TACGCGGATA AGACTTATCA
GCCAGAACCT CAGGTGGGCG AAGAAAGCTG GACAGACACA
GATGGAACTA ACGAAAAATT CGGAGGTAGA GCTTTGAAAC
CCGCCACTAA AATGAAACCA TGCTACGGTA GCTTCGCTCG
ACCTACCAAC ATTAAGGGAG GCCAAGCTAA AAATCGGAAA
GTGAAACCTA CAGAAGGCGA TGTGGAGACC GAGGAACCCG
ACATAGATAT GGAGTTTTTC GATGGTCGGG AAGCCGCCGA
TGCCTTTTCT CCAGAGATCG TGCTTTACAC GGAAAACGTT
AATCTGGAAA CTCCAGATAG CCACGTTGTG TACAAGCCAG
GAACTAGCGA CGACAACTCC CACGCCAACC TCGGCCAGCA
GGCAATGCCC AACAGACCTA ACTACATTGG ATTTAGAGAT
AACTTCGTGG GGCTTATGTA CTACAACTCC ACCGGCAACA
TGGGTGTCCT TGCAGGACAA GCTAGTCAGC TTAATGCAGT
GGTTGATCTC CAGGATCGCA ATACCGAATT GTCTTATCAA
CTGCTTTTGG ATAGTCTTGG GGACCGGACA AGGTATTTCT
CTATGTGGAA TCAGGCTGTG GATAGTTACG ACCCCGATGT
CCGGATCATT GAAAATCATG GGATTGAAGA CGAGCTGCCT
AATTATTGCT TTCCCCTTGA CGGCATCGGC CCAGCTAAGA
CATACCAGGG CATTAAAAGC AAAGACAACG GATGGGAGAA
GGATGATAAT GTATCAAAAT CTAATGAGAT CGCAATCGGC
AACAACCAGG CGATGGAAAT TAACATTCAG GCAAATCTGT
GGAGATCCTT TTTGTATAGC AATGTCGCGC TGTATTTGCC
AGACGTATAC AAGTACACTC CGACCAACAT AACCCTGCCC
GCTAATACTA ACACTTATGA ATACATGAAC GGACGAGTGG
TAAGCCCCAG CTTGGTGGAC AGTTACATCA ACATCGGCGC
CCGCTGGAGC CTTGATCCTA TGGATAACGT CAATCCCTTC
AATCACCATA GAAATGCAGG CCTGCGGTAT CGGTCTATGC
TTCTCGGCAA TGGGAGATAT GTCCCCTTTC ACATTCAAGT
GCCTCAGAAA TTCTTCGCAG TGAAAAACCT GCTTCTCTTG
CCGGGGTCCT ATACCTATGA ATGGAATTTC CGGAAGGATG
TCAACATGGT TCTGCAATCT AGCTTGGGAA ATGATCTTAG
AACGGACGGC GCTACCATCT CTTTCACCAG TATTAACCTT
TACGCTACTT TCTTTCCAAT GGCCCACAAC ACTGCATCCA
CCCTGGAGGC CATGCTCAGG AATGATACCA ATGACCAGAG
CTTTAATGAT TACCTCTCTG CAGCCAATAT GTTGTATCCG
ATCCCGGCGA ACGCCACCAA CATTCCCATC TCTATACCGT
CTCGCAATTG GGCCGCTTTT AGAGGGTGGA GCTTTACACG
GCTGAAGACT AAGGAGACCC CCTCCCTTGG CTCAGGATTT
GACCCTTACT TTGTGTACTC TGGGAGTATC CCCTATTTGG
ACGGCACTTT TTATCTGAAC CACACATTCA AGAAAGTTTC
TATCATGTTC GACTCTTCAG TTTCTTGGCC AGGAAACGAT
AGATTGCTTA GCCCCAACGA ATTTGAAATT AAACGCACAG
TCGACGGAGA AGGATATAAT GTCGCTCAGT GCAACATGAC
CAAAGATTGG TTTCTGGTGC AAATGCTCGC TAACTATAAC
ATAGGCTATC AGGGCTTCTA TATTCCTGAA GGCTATAAAG
ATAGGATGTA TTCATTTTTT AGGAACTTCC AACCAATGTC
TAGGCAAGTC GTGGACGAGG TAAACTACAC AGACTATAAG
GCCGTGACTC TCCCATATCA GCATAACAAT TCAGGCTTTG
TGGGATACTT GGCACCAACT ATGCGCCAGG GTGAGCCCTA
TCCCGCCAAC TACCCTTACC CCTTGATTGG TACAACTGCA
GTGAAGTCAG TGACCCAAAA AAAGTTTCTT TGCGATCGCA
CTATGTGGAG AATCCCTTTC TCCTCCAATT TCATGAGTAT
GGGAGCTCTC ACTGATCTCG GTCAGAACAT GTTGTATGCC
AATAGCGCAC ATGCGCTCGA TATGACATTT GAAGTTGATC
CGATGGACGA GCCTACCCTG CTCTATTTGC TGTTTGAGGT
TTTTGACGTC GTGAGAGTGC ACCAACCTCA TCGGGGCGTG
ATTGAAGCTG TATACTTGCG CACACCCTTC TCAGCCGGGA
ACGCTACCAC TTAA
Sequence Name: Hexon
Length: 937
Type: Amino Acids
Organism: Adenovirus 7
Other information: Description of sequence:
Synthetic
SEQ ID NO: 2
M A T P S M M P Q W A Y M H I A G Q D A S E
Y L S P G L V Q F A R A T D T Y F S M G N K
F R N P T V A P T H D V T T D R S Q R L M L
R F V P V D R E D N T Y S Y K V R Y T L A V
G D N R V L D M A S T F F D I R G V L D R G
P S F K P Y S G T A Y N S L A P K G A P N T
S Q W I V T A G E E R A V T T T T N T F G I
A S M K G D N I T K E G L E I G K D I T A D
N K P I Y A D K T Y Q P E P Q V G E E S W T
D T D G T N E K F G G R A L K P A T K M K P
C Y G S F A R P T N I K G G Q A K N R K V K
P T E G D V E T E E P D I D M E F F D G R E
A A D A F S P E I V L Y T E N V N L E T P D
S H V V Y K P G T S D D N S H A N L G Q Q A
M P N R P N Y I G F R D N F V G L M Y Y N S
T G N M G V L A G Q A S Q L N A V V D L Q D
R N T E L S Y Q L L L D S L G D R T R Y F S
M W N Q A V D S Y D P D V R I I E N H G I E
D E L P N Y C F P L D G I G P A K T Y Q G I
K S K D N G W E K D D N V S K S N E I A I G
N N Q A M E I N I Q A N L W R S F L Y S N V
A L Y L P D V Y K Y T P T N I T L P A N T N
T Y E Y M N G R V V S P S L V D S Y I N I G
A R W S L D P M D N V N P F N H H R N A G L
R Y R S M L L G N G R Y V P F H I Q V P Q K
F F A V K N L L L L P G S Y T Y E W N F R K
D V N M V L Q S S L G N D L R T D G A T I S
F T S I N L Y A T F F P M A H N T A S T L E
A M L R N D T N D Q S F N D Y L S A A N M L
Y P I P A N A T N I P I S I P S R N W A A F
R G W S F T R L K T K E T P S L G S G F D P
Y F V Y S G S I P Y L D G T F Y L N H T F K
K V S I M F D S S V S W P G N D R L L S P N
E F E I K R T V D G E G Y N V A Q C N M T K
D W F L V Q M L A N Y N I G Y Q G F Y I P E
G Y K D R M Y S F F R N F Q P M S R Q V V D
E V N Y T D Y K A V T L P Y Q H N N S G F V
G Y L A P T M R Q G E P Y P A N Y P Y P L I
G T T A V K S V T Q K K F L C D R T M W R I
P F S S N F M S M G A L T D L G Q N M L Y A
N S A H A L D M T F E V D P M D E P T L L Y
L L F E V F D V V R V H Q P H R G V I E A V
Y L R T P F S A G N A T T
Sequence Name: Penton
Length: 1635
Type: DNA
Organism: Adenovirus 7
Other information: Description of sequence:
Synthetic
SEQ ID NO: 3
ATGAGAAGAC GCGCCGTGCT TGGCGGAGCA ATGGTTTATC
CAGAAGGACC TCCCCCATCC TACGAGTCAG TAATGCAGCA
GCAAGCCGCC ATGATCCAAC CACCTCTTGA AGCTCCATTT
GTACCTCCGC GCTACTTGGC ACCGACTGAG GGCAGAAACT
CAATCCGCTA CTCAGAACTT AGCCCGCTCT ATGATACCAC
AAAACTGTAT CTCGTAGATA ACAAAAGTGC TGACATAGCT
TCACTCAACT ACCAAAATGA CCATTCCAAT TTTCTCACAA
CAGTGGTCCA AAACAACGAC TTTACTCCAA CAGAAGCAAG
TACACAGACA ATTAATTTTG ACGAAAGATC CCGCTGGGGG
GGCCAGCTGA AAACTATAAT GCATACAAAT ATGCCCAACG
TAAACGAGTA CATGTTCTCA AACAAGTTCA AGGCAAGGGT
GATGGTCTCT CGCAAGGCAC CCGAGGGCGT AATAGTCAAC
GATACGTATG ACCACAAAGA AGACATCCTG AAATACGAGT
GGTTTGAGTT CACACTGCCC GAGGGAAATT TTAGTGCAAC
AATGACTATT GACTTGATGA ATAATGCCAT TATCGACAAC
TACCTTGAAA TCGGAAGACA GAATGGGGTG CTCGAATCAG
ACATCGGAGT CAAATTCGAC ACACGGAACT TCAGACTGGG
CTGGGACCCC GAAACAAAAC TGATCATGCC AGGTGTGTAC
ACATATGAAG CGTTTCATCC TGACATAGTG TTGCTCCCCG
GATGCGGCGT CGACTTCACA GAATCCAGAT TGAGCAACCT
GCTTGGTATC AGAAAACGAC ATCCGTTTCA AGAAGGTTTC
AAAATAATGT ATGAGGATTT GGAAGGCGGT AATATCCCAG
CACTTTTGGA CGTAACAGCA TATGAAGAGA GCAAAAAAGA
CACTACCACT GAGACAACAA CACTGGCAGT CGCCGAGGAG
ACATCAGAGG ATGATAACAT CACACGCGGT GATACCTACA
TTACAGAGAA GCAAAAGAGA GAGGCAGCTG CCGCCGAAGT
GAAAAAAGAG CTCAAGATCC AACCATTGGA AAAGGATTCT
AAATCTCGAA GTTACAACGT TCTTGAAGAC AAAATCAATA
CAGCCTATCG CTCCTGGTAT TTGTCTTATA ACTATGGGAA
CCCGGAAAAA GGTATAAGAA GCTGGACCCT GCTGACGACA
TCTGATGTAA CATGTGGAGC TGAACAGGTT TACTGGAGTT
TGCCAGATAT GATGCAAGAC CCTGTGACAT TCCGAAGCAC
CCGACAGGTA AACAATTACC CCGTTGTGGG CGCCGAACTC
ATGCCCGTGT TCTCCAAGAG TTTTTACAAC GAACAGGCTG
TTTATTCCCA GCAATTGAGG CAGGCCACGT CCCTGACACA
CGTCTTCAAT AGATTTCCCG AAAATCAGAT CCTCATCCGG
CCCCCAGCCC CTACAATAAC CACAGTAAGC GAAAACGTGC
CAGCCTTGAC AGACCACGGC ACTCTCCCCC TGCGATCTTC
AATACGCGGC GTACAGCGAG TGACAGTTAC AGACGCTAGG
CGAAGGACTT GCCCTTATGT TTATAAGGCC CTCGGCATAG
TGGCCCCTAG AGTCCTGTCC TCTCGGACAT TCTAA
Sequence Name: Penton
Length: 544
Type: Amino Acids
Organism: Adenovirus 7
Other information: Description of sequence:
Synthetic
SEQ ID NO: 4
M R R R A V L G G A M V Y P E G P P P S Y E
S V M Q Q Q A A M I Q P P L E A P F V P P R
Y L A P T E G R N S I R Y S E L S P L Y D T
T K L Y L V D N K S A D I A S L N Y Q N D H
S N F L T T V V Q N N D F T P T E A S T Q T
I N F D E R S R W G G Q L K T I M H T N M P
N V N E Y M F S N K F K A R V M V S R K A P
E G V I V N D T Y D H K E D I L K Y E W F E
F T L P E G N F S A T M T I D L M N N A I I
D N Y L E I G R Q N G V L E S D I G V K F D
T R N F R L G W D P E T K L I M P G V Y T Y
E A F H P D I V L L P G C G V D F T E S R L
S N L L G I R K R H P F Q E G F K I M Y E D
L E G G N I P A L L D V T A Y E E S K K D T
T T E T T T L A V A E E T S E D D N I T R G
D T Y I T E K Q K R E A A A A E V K K E L K
I Q P L E K D S K S R S Y N V L E D K I N T
A Y R S W Y L S Y N Y G N P E K G I R S W T
L L T T S D V T C G A E Q V Y W S L P D M M
Q D P V T F R S T R Q V N N Y P V V G A E L
M P V F S K S F Y N E Q A V Y S Q Q L R Q A
T S L T H V F N R F P E N Q I L I R P P A P
T I T T V S E N V P A L T D H G T L P L R S
S I R G V Q R V T V T D A R R R T C P Y V Y
K A L G I V A P R V L S S R T F
Sequence Name: Fiber
Length: 978
Type: DNA
Organism: Adenovirus 7
Other information: Description of sequence:
Synthetic
SEQ ID NO: 5
ATGACCAAGC GGGTAAGACT TTCTGACAGT TTCAATCCTG
TCTACCCCTA CGAGGATGAG TCTACCAGTC AGCATCCCTT
CATAAACCCC GGATTCATCT CCCCCAATGG CTTTACACAA
TCTCCCGATG GCGTACTGAC TCTCAAGTGC TTGACCCCCC
TTACTACCAC AGGAGGTAGC CTTCAACTGA AAGTCGGCGG
AGGCTTGACT ATAGACGACA CAGACGGATT TCTCAAAGAA
AATATTAGCG CTGCAACCCC ATTGGTTAAA ACCGGACATA
GTATTGGACT TTCCCTGGGA CCTGGACTGG GAACAAACGA
AAATAAACTC TGCGCAAAGC TGGGTGAGGG CTTGACATTT
AATTCCAACA ATATTTGCAT AGATGACAAT ATTAACACAC
TTTGGACCGG AGTGAACCCA ACAACTGCTA ATTGTCAAAT
AATGGCCTCT TCAGAAAGTA ACGATTGCAA GTTGATTCTC
ACACTCGTCA AAACAGGCGC CCTTGTGACA GCCTTTGTTT
ATGTAATTGG GGTCTCAAAC GATTTTAACA TGCTCACAAC
TCACAAAAAC ATCAATTTCA CCGCCGAACT GTTCTTCGAT
AGCACAGGCA ACCTGTTGAC CTCCCTCAGC TCCCTTAAAA
CTCCCTTGAA TCACAAGTCT GGACAGAACA TGGCTACTGG
CGCTTTGACC AACGCAAAAG GTTTTATGCC CTCAACAACC
GCCTATCCCT TCAATGTGAA TAGTAGAGAA AAAGAAAACT
ATATTTATGG CACCTGTTAT TACACTGCAT CCGATCATAC
CGCCTTCCCT ATCGATATTT CTGTCATGCT GAATCAGAGA
GCCCTCAACA ATGAAACATC TTACTGCATA CGCGTCACCT
GGTCATGGAA TACAGGCGTG GCTCCCGAAG TTCAGACATC
CGCAACCACA CTCGTTACAT CACCATTCAC ATTCTATTAT
ATCAGGGAGG ACGATTAA
Sequence Name: Fiber
Length: 325
Type: Amino Acids
Organism: Adenovirus 7
Other information: Description of sequence:
Synthetic
SEQ ID NO: 6
M T K R V R L S D S F N P V Y P Y E D E S T
S Q H P F I N P G F I S P N G F T Q S P D G
V L T L K C L T P L T T T G G S L Q L K V G
G G L T I D D T D G F L K E N I S A A T P L
V K T G H S I G L S L G P G L G T N E N K L
C A K L G E G L T F N S N N I C I D D N I N
T L W T G V N P T T A N C Q I M A S S E S N
D C K L I L T L V K T G A L V T A F V Y V I
G V S N D F N M L T T H K N I N F T A E L F
F D S T G N L L T S L S S L K T P L N H K S
G Q N M A T G A L T N A K G F M P S T T A Y
P F N V N S R E K E N Y I Y G T C Y Y T A S
D H T A F P I D I S V M L N Q R A L N N E T
S Y C I R V T W S W N T G V A P E V Q T S A
T T L V T S P F T F Y Y I R E D D
Sequence Name: 100K
Length: 2490
Type: DNA
Organism: Adenovirus 7
Other information: Description of sequence:
Synthetic
SEQ ID NO: 7
ATGGAAACCC AGCCCAGCCT GCCAACCAGC CTCCAGGCCC
CTTCTCACCT CGCCCCTAGC AGCGACGAGG AAGAGAGCCT
GACCACCCCT CCACCTAGCC CTGCCACCAC CACCAGCACC
CTGGACGACG AAGAGGAAGA GGTGGACGCT GCTCAGGAAA
TGCAGGCCCA GGACAACGTG AAGGCCGAAG AGATCGAGGC
CGACGTGGAA CAGGACCCCG GCTACGTGAC CCCTGCCGAG
CACGAGGAAG AACTGAAGCG GTTCATCGAC AGAGAGGACG
ACGACCGGCC CGAGCACCAG GCCGATGGCG ATCACCAGGA
AGCCGGCATC GGCGACCAGG TGGCAGATTA CCTGACAGGC
CTGGGGGGCG AGGACGTGCT GCTGAAGCAC CTGGCCCGGC
AGAGCAACAT CGTGAAGGAC GCTCTGCTGG ACCTGACCGA
GGTGCCCATC AGCGTGGAAG AACTGTCCCG GGCCTACGAG
CTGAACCTGT TCAGCCCTCA CATCCCTCCC AAGCGGCAGC
CCAACGGCAC ATGCGAGGCC AACCCCAGAC TGAACTTCTA
CCCCGCCTTC ACCGTGCCCG AGGTGCTGGC CACCTACCAC
ATCTTTTTCA AGAACCAGAA AATCCCCGTG TCCTGCCGGG
CCAACCGGAC CAGAGCCGAT GCCCTGCTGA ATCTGGGCCC
TGGAGCCAGA CTGCCCGACA TTGCCAGCCT GGAAGAGGTG
CCCAAGATCT TCGAGGGCCT GGGCAGCGAC GAAACCAGAG
CCGCCAATGC TCTGCAGCAG GGCGAGAACG GCATGGACGA
GCACCACAGC GCCCTGGTGG AACTGGAAGG CGACAACGCC
CGGCTGGCCG TGCTGAAGCG GAGCATCGTG GTGACACACT
TCGCCTACCC CGCCGTGAAC CTGCCTCCCA AAGTGATGAG
CGCCGTGATG GACCATCTGC TGATCAAGAG AGCCGGCCCT
CTGAGCGAGA ACCAGAACAT GCAGGACCCC GACGCCTCCG
ACGAGGGCAA GCCTGTGGTG TCCGACGAGC AGCTGAGCCG
GTGGCTGGGC ACCAACAGCC CCAGGGACCT GGAAGAACGG
CGGAAGCTGA TGATGGCCGT GGTGCTGGTG ACAGTGGAAC
TGGAATGCCT GCGGCGGTTC TTCACCGACC CCGAGACACT
GCGGAAGCTG GAAGAGAACC TGCACTACAC CTTCCGGCAC
GGCTTCGTGC GGCAGGCTTG CAAGATCAGC AACGTGGAAC
TGACCAACCT GGTGTCCTAC ATGGGCATCC TGCACGAGAA
CCGGCTGGGC CAGAGCGTGC TGCACACCAC CCTGAAGGGC
GAGGCCAGAC GGGACTACAT CCGGGACTGC GTGTACCTGT
ACCTGTGCCA CACCTGGCAG ACCGGCATGG GCGTGTGGCA
GCAGTGTCTG GAAGAACAGA ACCTGAAAGA GCTGGACAAG
CTGCTGCAGC GGAGCCTGAA GGCCCTGTGG ACCGGCTTCG
ACGAGAGAAC CGTGGCCAGC GACCTGGCCG ACATCATCTT
CCCCGAGCGG CTGAGAGTGA CCCTGCGGAA CGGCCTGCCC
GACTTCATGA GCCAGAGCAT GCTGAACAAC TTCCGGTCCT
TCATCCTGGA AAGAAGCGGC ATTCTGCCCG CCACCTGTTG
CGCCCTGCCC AGCGATTTCG TGCCCCTGAC CTACAGAGAG
TGCCCTCCTC CCCTGTGGTC CCACTGCTAC CTGTTCCGGC
TGGCCAACTA CCTGAGCTAC CACAGCGACG TGATCCAGGA
CGTGTCCGGC GACGGCCTGC TGGAATGCCA CTGCCGGTGC
AATCTGTGCA CCCCACACAG AAGCCTGGCC TGCAACCCTC
AGCTGCTGAG CGAGACACAG ATCATCGGCA CATTCGAGCT
GCAAGGCCCC AGCTCTGAGG GCGAGGGAAG CTCTCCTGGC
CAGAGCCTGA AGCTGACCCC TGGCCTGTGG ACCAGCGCCT
ACCTGCGGAA GTTCCACCCC GAGGACTACC ACCCCTACGA
GATCCGGTTC TACGAGGACC AGTCCCACCC TCCCAAGGTG
GAACTGAGCG CCTGCGTCAT CACCCAGGGC GCCATTCTGG
CTCAGCTGCA GGCCATCCAG AAGTCCCGGC AGGAATTTCT
GCTGAAGAAA GGCAGCGGAG TGTACCTGGA CCCTCAGACC
GGCGAGGAAC TGAACACCCG GTTCCCTCAG GACGTGCCCA
GCCCCAGAAA GCAGGAAGCC GAGGGCGCTG CCGACGCCCC
TAGAGCATAT GGCGGCAGAC TGGGCCAGTC CGGCAGAGGC
GGAGATCGAA GGCTGGGACA GCCTGGCAGG GGCGGAGGAC
AGCCAGGCGG CAGACAGTTC GGCGGAGGCA GAAGGGGAGG
ACGCGGAGGC GGCAGAAGCA ACAGACGGCA GACCGTGGTG
ATCGGAGGCG GCGACAAACA GGTGCCCCGG CAGCAGCACG
GCTACCACCT GAGATCTGGC TCTGGCGGAC CTGCCGCCGC
TCAGCAGTGA
Sequence Name: 100K
Length: 828
Type: Amino Acids
Organism: Adenovirus 7
Other information: Description of sequence:
Synthetic
SEQ ID NO: 8
M E T Q P S L P T S L Q A P S H L A P S S D E
E E S L T T P P P S P A T T T S T L D D E E
E E V D A A Q E M Q A Q D N V K A E E I E A
D V E Q D P G Y V T P A E H E E E L K R F I
D R E D D D R P E H Q A D G D H Q E A G I G
D Q V A D Y L T G L G G E D V L L K H L A R
Q S N I V K D A L L D L T E V P I S V E E L
S R A Y E L N L F S P H I P P K R Q P N G T
C E A N P R L N F Y P A F T V P E V L A T Y
H I F F K N Q K I P V S C R A N R T R A D A
L L N L G P G A R L P D I A S L E E V P K I
F E G L G S D E T R A A N A L Q Q G E N G M
D E H H S A L V E L E G D N A R L A V L K R
S I V V T H F A Y P A V N L P P K V M S A V
M D H L L I K R A G P L S E N Q N M Q D P D
A S D E G K P V V S D E Q L S R W L G T N S
P R D L E E R R K L M M A V V L V T V E L E
C L R R F F T D P E T L R K L E E N L H Y T
F R H G F V R Q A C K I S N V E L T N L V S
Y M G I L H E N R L G Q S V L H T T L K G E
A R R D Y I R D C V Y L Y L C H T W Q T G M
G V W Q Q C L E E Q N L K E L D K L L Q R S
L K A L W T G F D E R T V A S D L A D I I F
P E R L R V T L R N G L P D F M S Q S M L N
N F R S F I L E R S G I L P A T C C A L P S
D F V P L T Y R E C P P P L W S H C Y L F R
L A N Y L S Y H S D V I E D V S G D G L L E
C H C R C N L C T P H R S L A C N P Q L L S
E T Q I I G T F E L Q G P S S E G E G S S P
G Q S L K L T P G L W T S A Y L R K F H P E
D Y H P Y E I R F Y E D Q S H P P K V E L S
A C V I T Q G A I L A Q L Q A I Q K S R Q E
F L L K K G S G V Y L D P Q T G E E L N T R
F P Q D V P S P R K Q E A E G A A D A P R G
Y G G R L G Q S G R G G D G R L G Q P G R G
G G Q P G G R Q F G G G R R G G R G G G R S
N R R Q T V V I G G G D K Q V P R Q Q H G Y
H L R S G S G G P A A A Q Q
Sequence Name: 52/55K
Length: 1158
Type: DNA
Organism: Adenovirus 7
Other information: Description of sequence:
PCR amplified
SEQ ID NO: 9
ATGCATCCAG TGCTGCGACA GATGCGCCCC CAGCAACAGG
CCCCTTCTCA GCAACAGCCA CAAAAGGCTC TTCTTGCTCC
TGCAACTACT GCAGCTGCAG CCGTGAGCGG CGCGGGACAG
CCCGCCTATG ATCTGGACTT GGAAGAGGGC GAGGGATTGG
CGCGCCTGGG GGCTCCATCG CCCGAGCGGC ACCCGCGGGT
GCAACTAAAA AAGGACTCTC GCGAGGCATA CGTGCCCCAG
CAGAACCTGT TTAGGGACAG GAGCGGCGAG GAGCCAGAGG
AGATGCGAGC ATCTCGATTT AACGCGGGTC GCGAGCTGCG
CCACGGTCTG GATCGAAGAC GGGTGCTGCA AGACGAGGAT
TTTGAGGTCG ATGAAGTCAC AGGGATCAGC CCAGCTAGGG
CACATGTGGC CGCGGCCAAC CTAGTCTCGG CCTACGAGCA
GACCGTGAAG GAGGAGCGCA ACTTCCAAAA ATCTTTTAAC
AACCATGTGC GCACCCTGAT TGCCCGCGAG GAAGTGACCC
TGGGTCTGAT GCATCTGTGG GACCTGATGG AGGCTATCGC
CCAAAACCCC ACTAGCAAAC CACTGACAGC TCAGCTGTTT
CTGGTGGTTC AACATAGCAG GGACAACGAG GCATTCAGGG
AGGCGTTGTT GAACATCACC GAGCCTGATG GGAGATGGCT
GTATGATCTG ATCAACATCC TGCAAAGTAT TATAGTGCAG
GAACGTAGCC TGGGTTTGGC TGAGAAAGTG GCAGCTATCA
ACTACTCGGT CTTGAGCCTG GGCAAATACT ACGCTCGCAA
GATCTACAAG ACCCCCTACG TACCCATAGA CAAGGAGGTG
AAGATAGATG GGTTTTACAT GCGCATGACT CTAAAGGTAC
TAACTCTAAG CGACGATCTG GGGGTGTATC GCAATGACAG
GATGCATCGC GCGGTGAGCG CCAGTAGGAG GCGCGAGCTG
AGCGACAGAG AACTTATGCA CAGCTTGCAA AGAGCTCTAA
CGGGGGCCGG GACTGATGGG GAGAACTACT TTGACATGGG
AGCGGACTTG CAATGGCAAC CCAGTCGCAG GGCCATGGAG
GCTGCAGGGT GTGAGCTTCC TTACATAGAA GAGGTGGATG
AAGTCGAGGA CGAGGAGGGC GAGTACTTGG AAGACTGA
Sequence Name: 52/55K
Length: 385
Type: Amino Acids
Organism: Adenovirus 7
Other information: Description of sequence:
PCR amplified
SEQ ID NO: 10
M H P V L R Q M R P Q Q Q A P S Q Q Q P Q K
A L L A P A T T A A A A V S G A G Q P A Y D
L D L E E G E G L A R L G A P S P E R H P R
V Q L K K D S R E A Y V P Q Q N L F R D R S
G E E P E E M R A S R F N A G R E L R H G L
D R R R V L Q D E D F E V D E V T G I S P A
R A H V A A A N L V S A Y E Q T V K E E R N
F Q K S F N N H V R T L I A R E E V T L G L
M H L W D L M E A I A Q N P T S K P L T A Q
L F L V V Q H S R D N E A F R E A L L N I T
E P D G R W L Y D L I N I L Q S I I V Q E R
S L G L A E K V A A I N Y S V L S L G K Y Y
A R K I Y K T P Y V P I D K E V K I D G F Y
M R M T L K V L T L S D D L G V Y R N D R M
H R A V S A S R R R E L S D R E L M H S L Q
R A L T G A G T D G E N Y F D M G A D L Q W
Q P S R R A M E A A G C E L P Y I E E V D E
V E D E E G E Y L E D
Sequence Name: IIIa
Length: 1767
Type: DNA
Organism: Adenovirus 7
Other information: Description of sequence:
Synthetic
SEQ ID NO: 11
ATGGAACAGC AGCAGGCCCC TGACCCTGCC ATGAGAGCCG
CCCTGCAGAG CCAGCCCAGC GGCATCAACA GCAGCGACGA
CTGGACCCAG GCCATGCAGC GGATCATGGC CCTGACCACC
CGGAACCCCG AGGCCTTCAG ACAGCAGCCC CAGGCCAACC
GGCTGAGCGC CATTCTGGAA GCCGTGGTGC CCAGCCGGTC
CAACCCCACC CACGAGAAGG TGCTGGCCAT CGTGAACGCC
CTGGTGGAAA ACAAGGCCAT CCGGCCCGAT GAGGCTGGCC
TGGTGTACAA TGCCCTGCTG GAACGGGTGG CCCGGTACAA
CTCCAGCAAC GTGCAGACCA ACCTGGACCG GATGGTGACA
GACGTGCGCG AGGCCGTGTC CCAGAGAGAG AGATTCCAGC
GGGACGCCAA CCTGGGCAGC CTGGTGGCCC TGAACGCCTT
CCTGAGCACC CAGCCCGCCA ACGTGCCCAG AGGCCAGCAG
GACTACACCA ACTTTCTGAG CGCCCTGCGG CTGATGGTGG
CCGAGGTGCC ACAGAGCGAG GTGTACCAGA GCGGCCCTGA
CTACTTCTTC CAGACCAGCC GGCAGGGACT GCAGACCGTG
AACCTGACCC AGGCCTTCAA GAACCTGAAG GGCCTGTGGG
GAGTGCACGC TCCCGTGGGA GATAGAGCCA CCGTGTCCAG
CCTGCTGACC CCTAACAGCA GACTCCTGCT GCTGCTGGTG
TCCCCATTCA CCGACAGCGG CTCCATCGAC CGGAACAGCT
ACCTGGGCTA CCTGCTGAAC CTGTACAGAG AGGCCATCGG
CCAGAGCCAG GTGGACGAGC AGACATACCA GGAAATCACA
CAGGTCTCCA GAGCCCTGGG CCAGGAAGAT ACCGGCAGCC
TGGAAGCCAC CCTGAACTTT CTGCTGACCA ACAGAAGCCA
GAAGATCCCT CCCCAGTACG CCCTGACCGC CGAGGAAGAA
AGAATCCTGA GATACGTGCA GCAGAGCGTG GGCCTGTTCC
TGATGCAAGA AGGCGCCACC CCTACAGCCG CCCTGGACAT
GACCGCCCGG AACATGGAAC CCTCTATGTA CGCCAGCAAC
CGGCCCTTCA TCAACAAACT GCTGGACTAC CTGCACCGGG
CTGCCGCCAT GAACAGCGAC TACTTCACCA ACGCCATCCT
GAACCCTCAC TGGCTGCCTC CACCCGGCTT CTACACCGGC
GAGTACGACA TGCCCGACCC CAACGACGGC TTCCTGTGGG
ACGACGTGGA CAGCAGCATC TTCAGCCCAC CTCCCGGCTA
CACCGTGTGG AAGAAAGAGG GCGGCGACAC CCGGCACAGC
AGCGTGTCAC TGAGCAGAAC AGCCGGCGCT GCCGCTACAG
TGCCTGAGGC CGCCAGCCCA TTCCCCAGCC TGCCCTTCAG
CCTGAACAGC GTGCGGAGCA GCGAGCTGGG CCGGATCACC
AGACCCAGAC TGATGGGCGA GGACGAGTAC CTGAACGACA
GCCTGCTGCG GCCCGAGAGA GAGAAGAACT TCCCCAACAA
CGGCATCGAG AGCCTGGTGG ACAAGATGAG CCGGTGGAAA
ACCTACGCCC AGGACCACCG GGACGAGCCC AAGATCCTGG
GCGCCACCAG CCGGCCCACC AGAAGAAGGC AGCGGCACGA
CAGACAGATG GGCCTGGTCT GGGATGATGA GGACTCCGCC
GACGACAGCT CCGTGCTGGA TCTGGGCGGC AGAGGCGGCG
GAAACCCTTT CGCTCACCTG AGGCCCAGAT TCGGCCGGAT
GCTGTGA
Sequence Name: IIIa
Length: 588
Type: Amino Acids
Organism: Adenovirus 7
Other information: Description of sequence:
Synthetic
SEQ ID NO: 12
M E Q Q Q A P D P A M R A A L Q S Q P S G I
N S S D D W T Q A M Q R I M A L T T R N P E
A F R Q Q P Q A N R L S A I L E A V V P S R
S N P T H E K V L A I V N A L V E N K A I R
P D E A G L V Y N A L L E R V A R Y N S S N
V Q T N L D R M V T D V R E A V S Q R E R F
Q R D A N L G S L V A L N A F L S T Q P A N
V P R G Q Q D Y T N F L S A L R L M V A E V
P Q S E V Y Q S G P D Y F F Q T S R Q G L Q
T V N L T Q A F K N L K G L W G V H A P V G
D R A T V S S L L T P N S R L L L L L V S P
F T D S G S I D R N S Y L G Y L L N L Y R E
A I G Q S Q V D E Q T Y Q E I T Q V S R A L
G Q E D T G S L E A T L N F L L T N R S Q K
I P P Q Y A L T A E E E R I L R Y V Q Q S V
G L F L M Q E G A T P T A A L D M T A R N M
E P S M Y A S N R P F I N K L L D Y L H R A
A A M N S D Y F T N A I L N P H W L P P P G
F Y T G E Y D M P D P N D G F L W D D V D S
S I F S P P P G Y T V W K K E G G D T R H S
S V S L S R T A G A A A T V P E A A S P F P
S L P F S L N S V R S S E L G R I T R P R L
M G E D E Y L N D S L L R P E R E K N F P N
N G I E S L V D K M S R W K T Y A Q D H R D
E P K I L G A T S R P T R R R Q R H D R Q M
G L V W D D E D S A D D S S V L D L G G R G
G G N P F A H L R P R F G R M L
Sequence Name: VI
Length: 753
Type: DNA
Organism: Adenovirus 7
Other information: Description of sequence:
Synthetic
SEQ ID NO: 13
ATGGAAGATA TTAACTTCAG CTCCCTGGCC CCTCGGCACG
GCACCAGACC CTACATGGGC ACTTGGAGCG ACATCGGCAC
CAGCCAGCTG AATGGCGGAG CCTTCAACTG GTCCAGCATT
TGGAGCGGCC TGAAGAACTT CGGCTCCACC GTGAAAACCT
ACGGCAACAA GGCCTGGAAC AGCTCCACCG GCCAGGCCCT
GCGGAACAAG CTGAAAGAGC AGAACTTCCA GCAGAAAGTG
GTGGACGGAA TCGCCAGCGG CATCAACGGC GTGGTGGACC
TGGCCAACCA GGCCGTGCAG AAGCAGATCA ACTCCCGGCT
GGACCCTCCT CCCGCCACCC CTGGCGAGAT GGAAGTGGAA
GAGGAACTGC CTCCCCTGGA AAAGCGGGGC GACAAGAGGC
CCAGACCCGA ACTGGAACAG ACCCTGGTGA CACGGGCCGA
CGAGCCTCCC AGCTACGAGG AAGCCGTGAA GCTGGGCATG
CCTACCACCA GACCCGTGGC CCACATGGCC ACCGGCGTGA
TGAAGCCCAG CCAGAGCCAC AGACCCGCCA CCCTGGATCT
GCCTCCACCT CCTGCCTCTG CCGCTCTGGT GCCTAAGCCC
GTGGCCACCA GAAAGCCCAC CGCCGTGCAG CCTGTGGCCG
TGGCTAGACC TAGACCTGGC GGCACCCCTA GGCCAAAGGC
CAACTGGCAG AGCACCCTGA ACAGCATCGT GGGCCTGGGC
GTGCAGAGCG TGAAGCGGCG GAGATGCTAC TGA
Sequence Name: VI
Length: 250
Type: Amino Acids
Organism: Adenovirus 7
Other information: Description of sequence:
Synthetic
SEQ ID NO: 14
M E D I N F S S L A P R H G T R P Y M G T W
S D I G T S Q L N G G A F N W S S I W S G L
K N F G S T V K T Y G N K A W N S S T G Q A
L R N K L K E Q N F Q Q K V V D G I A S G I
N G V V D L A N Q A V Q K Q I N S R L D P P
P A T P G E M E V E E E L P P L E K R G D K
R P R P E L E Q T L V T R A D E P P S Y E E
A V K L G M P T T R P V A H M A T G V M K P
S Q S H R P A T L D L P P P P A S A A L V P
K P V A T R K P T A V Q P V A V A R P R P G
G T P R P K A N W Q S T L N S I V G L G V Q
S V K R R R C Y
Sequence Name: VIII
Length: 684
Type: DNA
Organism: Adenovirus 7
Other information: Description of sequence:
Synthetic
SEQ ID NO: 15
ATGAGCAAAG AGATCCCCAC CCCTTACATG TGGTCCTACC
AGCCCCAGAT GGGCCTGGCC GCTGGCGCCT CTCAGGACTA
CAGCACCCGG ATGAACTGGC TGAGCGCCGG ACCCAGCATG
ATCAGCAGAG TGAACGACAT CCGGGCCTAC CGGAACCAGC
TGCTGCTGGA ACAGAGCGCC CTGACCACCA CCCCTCGGCA
GCACCTGAAC CCCAGAAACT GGCCTGCCGC CCTGGTGTAC
CAGGAAACCC CTGCCCCTAC CACCGTGCTG CTGCCCAGAG
ATGCCCAGGC CGAGGTGCAC ATGACCAACG CTGGCGTGCA
GCTGGCTGGC GGCTCTGCCC TGTGCAGACA CAGACCCCAG
CAGAGCATCA AGCGGCTGGT GATCCGGGGC AGAGGCATCC
AGCTGAACGA CGAGAGCGTG TCCAGCAGCC TGGGACTGAG
GCCCGACGGC GTGTTCCAGA TCGCCGGCTG CGGCAGAAGC
AGCTTCACCC CTAGACAGGC CGTGCTGACC CTGGAAAGCA
GCAGCAGCCA GCCCAGAAGC GGCGGCATCG GCACCCTGCA
GTTCGTGGAA GAGTTCACCC CAAGCGTGTA CTTCAACCCC
TTCAGCGGCA GCCCTGGCCA GTACCCCGAC GAGTTCATCC
CCAACTTCGA CGCCATCAGC GAGAGCGTGG ACGGCTACGA
CTGA
Sequence Name: VIII
Length: 227
Type: Amino Acids
Organism: Adenovirus 7
Other information: Description of sequence:
Synthetic
SEQ ID NO: 16
M S K E I P T P Y M W S Y Q P Q M G L A A G
A S Q D Y S T R M N W L S A G P S M I S R V
N D I R A Y R N Q L L L E Q S A L T T T P R
Q H L N P R N W P A A L V Y Q E T P A P T T
V L L P R D A Q A E V H M T N A G V Q L A G
G S A L C R H R P Q Q S I K R L V I R G R G
I Q L N D E S V S S S L G L R P D G V F Q I
A G C G R S S F T P R Q A V L T L E S S S S
Q P R S G G I G T L Q F V E E F T P S V Y F
N P F S G S P G Q Y P D E F I P N F D A I S
E S V D G Y D
Sequence Name: IX
Length: 417
Type: DNA
Organism: Adenovirus 7
Other information: Description of sequence:
Synthetic
SEQ ID NO: 17
ATGAGCGGCT CTGCCAGCTT TGAGGGGGGC GTGTTCAGCC
CCTACCTGAC CGGCAGACTG CCTCCTTGGG CTGGCGTGCG
GCAGAACGTG ATGGGCAGCA CCGTGGACGG CAGACCCGTG
CAGCCTGCCA ACAGCAGCAC CCTGACCTAC GCTACCCTGA
GCAGCAGCCC TCTGGATGCC GCTGCCGCCG CTGCTGCCAC
AGCCGCCGCT AATACCATCC TGGGCATGGG CTACTACGGC
TCTATCGTGG CCAACAGTTC CTCCAGCAAC AACCCCAGCA
CCCTGGCCGA GGACAAGCTG CTGGTGCTGC TGGCCCAGCT
GGAAGCCCTG ACCCAGAGAC TGGGCGAGCT GAGCAAACAG
GTGGCCCAGC TGAGAGAGCA GACCGAGTCT GCCGTGGCCA
CCGCCAAGAG CAAGTGA
Sequence Name: IX
Length: 138
Type: Amino Acids
Organism: Adenovirus 7
Other information: Description of sequence:
Synthetic
SEQ ID NO: 18
M S G S A S F E G G V F S P Y L T G R L P P
W A G V R Q N V M G S T V D G R P V Q P A N
S S T L T Y A T L S S S P L D A A A A A A A
T A A A N T I L G M G Y Y G S I V A N S S S
S N N P S T L A E D K L L V L L A Q L E A L
T Q R L G E L S K Q V A Q L R E Q T E S A V
A T A K S K

In accordance with one or more embodiments of the present application, exemplary AdVLPs, expression plasmids, compositions, and methods are set out in the following items:
Item 1. An adenovirus virus-like particle (AdVLP) comprising:
a recombinant capsid comprising:
   a) major capsid adenovirus (AdV) proteins including a hexon protein, a penton protein, and a fiber protein,
   b) minor capsid/cement AdV proteins, including a IIIa protein, a VI protein, a VIII protein, and a IX protein, wherein the minor capsid/cement AdV proteins structurally support the major capsid AdV proteins,
   c) a chaperone AdV protein L4-100k, and
   d) an accessory scaffold AdV protein L1-52/55k.
      Item 2. The AdVLP of item 1, wherein the AdVLP is an adenovirus type 4 virus-like particle (AdVLP4), adenovirus type 7 virus-like particle (AdVLP7), adenovirus type 14 virus-like particle (AdVLP14), or adenovirus type 55 virus-like particle (AdVLP55).
      Item 3. The AdVLP of item 1, wherein the major capsid AdV hexon protein comprises SEQ ID NO: 1 or SEQ ID NO: 2, the major capsid AdV penton protein comprises SEQ ID NO: 3 or SEQ ID NO: 4, and the major capsid AdV fiber protein comprises SEQ ID NO: 5 or SEQ ID NO: 6.
      Item 4. The AdVLP of any of items 1-3, wherein the minor capsid/cement Adv protein IIIA protein comprises SEQ ID NO: 11 or SEQ ID NO: 12, the minor capsid/cement Adv protein VI protein comprises protein comprises SEQ ID NO: 13 or SEQ ID NO: 14, the minor capsid/cement Adv protein VIII protein comprises protein comprises SEQ ID NO: 15 or SEQ ID NO: 16, and the minor capsid/cement Adv protein IX protein comprises protein comprises SEQ ID NO: 17 or SEQ ID NO: 18.
      Item 5. The AdVLP of any one of items 1-4, wherein the chaperone AdV protein L4-100k comprises SEQ ID NO: 7 or SEQ ID NO: 8.
      Item 6. The AdVLP of any one of items 1-5, wherein the accessory scaffold AdV L1-52/55k protein comprises SEQ ID NO: 9 or SEQ ID NO: 10.
      Item 7. The AdVLP of any one of items 1-6, further comprising an adjuvant.
      Item 8. The AdVLP of item 7, wherein the adjuvant is aluminum hydroxide or a squalene-based oil-in-water nano-emulsion.
      Item 9. An expression plasmid comprising genes encoding adenovirus proteins, wherein the expression plasmid is suitable for the assembly of adenovirus virus-like particles (AdVLPs) and wherein the expression plasmid comprises codon-optimized genes encoding:
a) major capsid adenovirus (AdV) proteins including a hexon protein, a penton protein, and a fiber protein,
b) minor capsid/cement AdV proteins, including a IIIa protein, a VI protein, a VIII protein, and a IX protein,
c) a chaperone AdV protein L4-100k, and
d) an accessory scaffold AdV protein L1-52/55k.
   Item 10. The expression plasmid of item 9, wherein the AdVLP is an adenovirus type 4 virus-like particle (AdVLP4), adenovirus type 7 virus-like particle (AdVLP7), adenovirus type 14 virus-like particle (AdVLP14), or adenovirus type 55 virus-like particle (AdVLP55).
   Item 11. The expression plasmid of item 9, wherein:
a) the major capsid AdV hexon protein comprises SEQ ID NO: 1 or SEQ ID NO: 2, the major capsid AdV penton protein comprises SEQ ID NO: 3 or SEQ ID NO: 4, and the major capsid AdV fiber protein comprises SEQ ID NO: 5 or SEQ ID NO: 6;
b) the minor capsid/cement Adv protein IIIA protein comprises SEQ ID NO: 11 or SEQ ID NO: 12, the minor capsid/cement Adv protein VI protein comprises protein comprises SEQ ID NO: 13 or SEQ ID NO: 14, the minor capsid/cement Adv protein VIII protein comprises protein comprises SEQ ID NO: 15 or SEQ ID NO: 16, and the minor capsid/cement Adv protein IX protein comprises protein comprises SEQ ID NO: 17 or SEQ ID NO: 18;
c) the chaperone AdV protein L4-100k comprises SEQ ID NO: 7 or SEQ ID NO: 8; and
d) the accessory scaffold AdV protein L1-52/55k comprises SEQ ID NO: 9 or SEQ ID NO: 10.
   Item 12. An immunogenic composition comprising at least one AdVLP of item 1.
   Item 13. The immunogenic composition of item 12, wherein the at least one AdVLP is adenovirus type 4 virus-like particle (AdVLP4), adenovirus type 7 virus-like particle (AdVLP7), adenovirus type 14 virus-like particle (AdVLP14), or adenovirus type 55 virus-like particle (AdVLP55).
   Item 14. The immunogenic composition of item 12, wherein the recombinant capsid of the at least one AdVLP comprises:
a) the major capsid AdV hexon protein comprises SEQ ID NO: 1 or SEQ ID NO: 2, the major capsid AdV penton protein comprises SEQ ID NO: 3 or SEQ ID NO: 4, and the major capsid AdV fiber protein comprises SEQ ID NO: 5 or SEQ ID NO: 6;
b) the minor capsid/cement Adv protein IIIA protein comprises SEQ ID NO: 11 or SEQ ID NO: 12, the minor capsid/cement Adv protein VI protein comprises protein comprises SEQ ID NO: 13 or SEQ ID NO: 14, the minor capsid/cement Adv protein VIII protein comprises protein comprises SEQ ID NO: 15 or SEQ ID NO: 16, and the minor capsid/cement Adv protein IX protein comprises protein comprises SEQ ID NO: 17 or SEQ ID NO: 18;
c) the chaperone AdV protein L4-100k comprises SEQ ID NO: 7 or SEQ ID NO: 8; and
d) the accessory scaffold AdV protein L1-52/55k comprises SEQ ID NO: 9 or SEQ ID NO: 10.
   Item 15. The immunogenic composition of item 13, wherein the composition is a trivalent composition comprising AdVLP4, AdVLP7, and AdVLP14.
   Item 16. The immunogenic composition of item 13, wherein the composition is a quadrivalent composition comprising AdVLP4, AdVLP7, AdVLP14, and AdVLP55.
   Item 17. The immunogenic composition of any one of items 12-16, wherein the composition is suitable for oral, nasal, mucosal, or parenteral administration.
   Item 18. A method of generating an immune response to one or more adenoviruses in a subject, the method comprising administering an effective amount of the immunogenic composition of claim 12 to the subject.
   Item 19. The method of item 18, wherein the one or more adenoviruses are selected from the group consisting of HAdV-E type 4, HAdV-B type 7, HAdV-B type 14, and HAdV-B type 55.
   Item 20. The method of item 18 or 19, wherein the immune response vaccinates the subject against the one or more adenoviruses.
   Item 21. The method of item 18, wherein the subject is a human.
   Item 22. A method of producing an adenovirus virus-like particle (AdVLP) comprising:
introducing into a host cell at least one expression plasmid under conditions such that the host cell produces the AdVLP, wherein the at least one expression plasmid comprises codon-optimized genes encoding:
   a) major capsid adenovirus (AdV) proteins including a hexon protein, a penton protein, and a fiber protein,
   b) minor capsid/cement AdV proteins, including a IIIa protein, a VI protein, a VIII protein, and a IX protein,
   c) a chaperone AdV protein L4-100k, and
   d) an accessory scaffold AdV protein L1-52/55k.
      Item 23. The method of item 22, wherein:
a) the major capsid AdV hexon protein comprises SEQ ID NO: 1 or SEQ ID NO: 2, the major capsid AdV penton protein comprises SEQ ID NO: 3 or SEQ ID NO: 4, and the major capsid AdV fiber protein comprises SEQ ID NO: 5 or SEQ ID NO: 6;
b) the minor capsid/cement Adv protein IIIA protein comprises SEQ ID NO: 11 or SEQ ID NO: 12, the minor capsid/cement Adv protein VI protein comprises protein comprises SEQ ID NO: 13 or SEQ ID NO: 14, the minor capsid/cement Adv protein VIII protein comprises protein comprises SEQ ID NO: 15 or SEQ ID NO: 16, and the minor capsid/cement Adv protein IX protein comprises protein comprises SEQ ID NO: 17 or SEQ ID NO: 18;
c) the chaperone AdV protein L4-100k comprises SEQ ID NO: 7 or SEQ ID NO: 8; and
d) the accessory scaffold AdV protein L1-52/55k comprises SEQ ID NO: 9 or SEQ ID NO: 10.
   Item 24. The method of item 22 or 23, wherein in the host cell is a eukaryotic cell.
   Item 25. The method of item 24, wherein the eukaryotic cell is a mammalian cell.
   Item 26. The method of item 22 or 23, further comprising the step of purifying the AdVLP.
   Item 27. The method of item 26, wherein the AdVLP is purified from cell lysates and culture supernatants.
   Item 28. The AdVLP of item 1, wherein the AdVLP comprises hexon proteins from two different serotypes such that the AdVLP is chimeric.
   Item 29. The AdVLP of item 1, wherein the hexon protein comprises one or more antigens of a different infectious agent.
   Item 30. The AdVLP of item 29, wherein the different infectious agent is SARS-CoV-2.

All publications, patents, and patent documents are incorporated by reference herein in their respective entireties, as though individually incorporated by reference. This statement of incorporation by reference is intended by applicants, pursuant to 37 C.F.R. § 1.57(b)(1), to relate to each and every individual publication, patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. No limitations inconsistent with this disclosure are to be understood therefrom.

The invention has been described with reference to various specific and preferred embodiments and techniques. However, it should be understood that many variations and modifications may be made while remaining within the spirit and scope of the invention.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims. Such equivalents are intended to be encompassed by the following claims.

### REFERENCES

1. Wold, W. S. M. & Toth, K. Adenovirus Vectors for Gene Therapy, Vaccination and Cancer Gene Therapy. Curr. Gene Ther. 13, 421-433 (2013).
2. Watanabe, M., Nishikawaji, Y., Kawakami, H. & Kosai, K. Adenovirus Biology, Recombinant Adenovirus, and Adenovirus Usage in Gene Therapy. Viruses 13, 2502 (2021).
3. Lion, T. Adenovirus Infections in Immunocompetent and Immunocompromised Patients. Clin. Microbiol. Rev. 27, 441-462 (2014).
4. Lin, K.-H. et al. A two decade survey of respiratory adenovirus in Taiwan: The reemergence of adenovirus types 7 and 4. J. Med. Virol. 73, 274-279 (2004).
5. Li, Q., Zheng, Q., Liu, Y. & Wadell, G. Molecular epidemiology of adenovirus types 3 and 7 isolated from children with pneumonia in Beijing. J. Med. Virol. 49, 170-177 (1996).
6. Sanchez, J. L. et al. Epidemic of adenovirus-induced respiratory illness among US military recruits: Epidemiologic and immunologic risk factors in healthy, young adults*†. J. Med. Virol. 65, 710-718 (2001).
7. Civilian Outbreak of Adenovirus Acute Respiratory Disease—South Dakota, 1997. https://www.cdc.gov/mmwr/preview/mmwrhtml/00053922.htm.
8. Ryan, M. A. K. et al. Large Epidemic of Respiratory Illness Due to Adenovirus Types 7 and 3 in Healthy Young Adults. Clin. Infect. Dis. 34, 577-582 (2002).
9. Shieh, W.-J. Human adenovirus infections in pediatric population—An update on clinico-pathologic correlation. Biomed. J. 45, 38-49 (2022).
10. Dudding, B. A. et al. Acute respiratory disease in military trainees: the adenovirus surveillance program, 1966-1971. Am. J. Epidemiol. 97, 187-198 (1973).
11. Dudding, B. A. et al. Fatal Pneumonia Associated with Adenovirus Type 7 in Three Military Trainees. N. Engl. J. Med. 286, 1289-1292 (1972).
12. Cui, X. et al. Human Adenovirus Type 7 Infection Associated with Severe and Fatal Acute Lower Respiratory Illness and Nosocomial Transmission. J. Clin. Microbiol. 53, 746-749 (2015).
13. Erdman, D. D. et al. Molecular Epidemiology of Adenovirus Type 7 in the United States, 1966-2000. Emerg. Infect. Dis. 8, 269-277 (2002).
14. Scott, M. K. et al. Human Adenovirus Associated with Severe Respiratory Infection, Oregon, USA, 2013-2014—Volume 22, Number 6—June 2016—Emerging Infectious Diseases journal—CDC. doi:10.3201/eid2206.151898.
15. Rozwadowski, F. Notes from the Field: Fatalities Associated with Human Adenovirus Type 7 at a Substance Abuse Rehabilitation Facility—New Jersey, 2017. MMWR Morb. Mortal. Wkly. Rep. 67, (2018).
16. Top, F. H. Control of adenovirus acute respiratory disease in U.S. Army trainees. Yale J. Biol. Med. 48, 185-195 (1975).
17. Zou, L. et al. Adenovirus infection in children hospitalized with pneumonia in Guangzhou, China. Influenza Other Respir. Viruses 15, 27-33 (2021).
18. Gavin, P. J. & Katz, B. Z. Intravenous Ribavirin Treatment for Severe Adenovirus Disease in Immunocompromised Children. Pediatrics 110, e9 (2002).
19. Gaydos, C. A. & Gaydos, J. C. Adenovirus Vaccines in the U.S. Military. Mil. Med. 160, 300-304 (1995).
20. Top, F. H., Jr. et al. Immunization with Live Types 7 and 4 Adenovirus Vaccines. I. Safety, Infectivity, Antigenicity, and Potency of Adenovirus Type 7 Vaccine in Humans. J. Infect. Dis. 124, 148-154 (1971).
21. Top, F. H., Jr., Buescher, E. L., Bancroft, W. H. & Russell, P. K. Immunization with Live Types 7 and 4 Adenovirus Vaccines. II. Antibody Response and Protective Effect against Acute Respiratory Disease Due to Adenovirus Type 7. J. Infect. Dis. 124, 155-160 (1971).
22. Collins, N. D. et al. Live Oral Adenovirus Type 4 and Type 7 Vaccine Induces Durable Antibody Response. Vaccines 8, 411 (2020).
23. Lyons, A. et al. A double-blind, placebo-controlled study of the safety and immunogenicity of live, oral type 4 and type 7 adenovirus vaccines in adults. Vaccine 26, 2890-2898 (2008).
24. Kuschner, R. A. et al. A phase 3, randomized, double-blind, placebo-controlled study of the safety and efficacy of the live, oral adenovirus type 4 and type 7 vaccine, in U.S. military recruits. Vaccine 31, 2963-2971 (2013).
25. Adenovirus Vaccine Information Statement|CDC. https://www.cdc.gov/vaccines/hcp/vis/vis-statements/adenovirus.html (2022).
26. Lukashev, A. N., Ivanova, O. E., Eremeeva, T. P. & Iggo, R. D. Evidence of frequent recombination among human adenoviruses. J. Gen. Virol. 89, 380-388 (2008).
27. Robinson, C. M. et al. Molecular evolution of human adenoviruses. Sci. Rep. 3, 1812 (2013).
28. Russell, K. L. et al. Vaccine-preventable adenoviral respiratory illness in US military recruits, 1999-2004. Vaccine 24, 2835-2842 (2006).
29. Mohsen, M. O. & Bachmann, M. F. Virus-like particle vaccinology, from bench to bedside. Cell. Mol. Immunol. 19, 993-1011 (2022).
30. Joura, E. A. et al. Efficacy of a quadrivalent prophylactic human papillomavirus (types 6, 11, 16, and 18) L1 virus-like-particle vaccine against high-grade vulval and vaginal lesions: a combined analysis of three randomised clinical trials. The Lancet 369, 1693-1702 (2007).
31. Drolet, M. et al. Population-level impact and herd effects following human papillomavirus vaccination programmes: a systematic review and meta-analysis. Lancet Infect. Dis. 15, 565-580 (2015).
32. Valenzuela, P., Medina, A., Rutter, W. J., Ammerer, G. & Hall, B. D. Synthesis and assembly of hepatitis B virus surface antigen particles in yeast. Nature 298, 347-350 (1982).
33. Keating, G. M. & Noble, S. Recombinant Hepatitis B Vaccine (Engerix-B®): A Review of its Immunogenicity and Protective Efficacy Against Hepatitis B. Drugs 63, 1021-1051 (2003).
34. Zhang, J. et al. Long-Term Efficacy of a Hepatitis E Vaccine. N. Engl. J. Med. 372, 914-922 (2015).
35. Nooraei, S. et al. Virus-like particles: preparation, immunogenicity and their roles as nanovaccines and drug nanocarriers. J. Nanobiotechnology 19, 59 (2021).
36. Reddy, V. S. & Nemerow, G. R. Structures and organization of adenovirus cement proteins provide insights into the role of capsid maturation in virus entry and infection. Proc. Natl. Acad. Sci. 111, 11715-11720 (2014).
37. Gallardo, J., Pérez-IIIana, M., Martin-González, N. & San Martin, C. Adenovirus Structure: What Is New?Int. J. Mol. Sci. 22, 5240 (2021).
38. Sumida, S. M. et al. Neutralizing Antibodies to Adenovirus Serotype 5 Vaccine Vectors Are Directed Primarily against the Adenovirus Hexon Protein1. J. Immunol. 174, 7179-7185 (2005).
39. Cheng, C. et al. Differential Specificity and Immunogenicity of Adenovirus Type 5 Neutralizing Antibodies Elicited by Natural Infection or Immunization. J. Virol. 84, 630-638 (2010).
40. Bradley, R. R., Lynch, D. M., lampietro, M. J., Borducchi, E. N. & Barouch, D. H. Adenovirus Serotype 5 Neutralizing Antibodies Target both Hexon and Fiber following Vaccination and Natural Infection. J. Virol. 86, 625-629 (2012).
41. Hu, K. et al. Penton base induces better protective immune responses than fiber and hexon as a subunit vaccine candidate against adenoviruses. Vaccine 36, 4287-4297 (2018).
42. Veen, J. V. D., Abarbanel, M. F. W. & Oei, K. G. Vaccination with live type 4 adenovirus: evaluation of antibody response and protective efficacy. Epidemiol. Infect. 66, 499-511 (1968).
43. Kolavic-Gray, S. A. et al. Large Epidemic of Adenovirus Type 4 Infection among Military Trainees: Epidemiological, Clinical, and Laboratory Studies. Clin. Infect. Dis. 35, 808-818 (2002).
44. Hasson, T. B., Ornelles, D. A. & Shenk, T. Adenovirus L1 52- and 55-kilodalton proteins are present within assembling virions and colocalize with nuclear structures distinct from replication centers. J. Virol. 66, 6133-6142 (1992).
45. Zandvliet, M. L. et al. Combined CD8+ and CD4+ adenovirus hexon-specific T cells associated with viral clearance after stem cell transplantation as treatment for adenovirus infection. Haematologica 95, 1943-1951 (2010).
46. Molloy, C. T. et al. Contributions of CD8 T cells to the pathogenesis of mouse adenovirus type 1 respiratory infection. Virology 507, 64-74 (2017).
47. Chakrabarti, S. et al. Adenovirus infections following allogeneic stem cell transplantation: incidence and outcome in relation to graft manipulation, immunosuppression, and immune recovery. Blood 100, 1619-1627 (2002).
48. Liao, J. et al. Severe pneumonia caused by adenovirus 7 in pregnant woman: Case report and review of the literature. J. Obstet. Gynaecol. Res. 42, 1194-1197 (2016).
49. Molnárová, A. et al. Adenovirus and RSV infections during pregnancy and their relationship to orofacial clefts. Biologia (Bratisl.) 75, 1055-1061 (2020).
50. Zhao, H. et al. A fatal case of viral sepsis and encephalitis in a child caused by human adenovirus type 7 infection. Virol. J. 19, 154 (2022).
51. Patel, N. et al. Acute Hepatitis of Unknown Origin in Pediatric Age Group: Recent Outbreaks and Approach to Management. J. Clin. Med. 12, 9 (2023).
52. MacNeil, K. M. et al. Adenoviruses in medicine: innocuous pathogen, predator, or partner. Trends Mol. Med. 29, 4-19 (2023).
53. Gustin, K. E. & Imperiale, M. J. Encapsidation of Viral DNA Requires the Adenovirus L1 52/55-Kilodalton Protein. J. Virol. 72, 7860-7870 (1998).
54. Weber, J. Genetic analysis of adenovirus type 2 III. Temperature sensitivity of processing viral proteins. J. Virol. 17, 462-471 (1976).
55. Mangel, W. F., Toledo, D. L., Brown, M. T., Martin, J. H. & McGrath, W. J. Characterization of Three Components of Human Adenovirus Proteinase Activity in Vitro(*). J. Biol. Chem. 271, 536-543 (1996).
56. Pérez-Berná, A. J. et al. Processing of the L1 52/55k Protein by the Adenovirus Protease: a New Substrate and New Insights into Virion Maturation. J. Virol. 88, 1513-1524 (2014).
57. Mangel, W. F. & San Martin, C. Structure, Function and Dynamics in Adenovirus Maturation. Viruses 6, 4536-4570 (2014).
58. Ishibashi, M. & Maizel, J. V. The polypeptides of adenovirus: V. Young virions, structural intermediate between top components and aged virions. Virology 57, 409-424 (1974).
59. Hannan, C., Raptis, L. H., D6ry, C. V. & Weber, J. Biological and Structural Studies with an Adenovirus Type 2 Temperature-Sensitive Mutant Defective for Uncoating. Intervirology 19, 213-223(1983).
60. Mangel, W. F., McGrath, W. J., Toledo, D. L. & Anderson, C. W. Viral DNA and a viral peptide can act as cofactors of adenovirus virion proteinase activity. Nature 361, 274-275 (1993).
61. Blainey, P. C. et al. Regulation of a Viral Proteinase by a Peptide and DNA in One-dimensional Space: IV. VIRAL PROTEINASE SLIDES ALONG DNA TO LOCATE AND PROCESS ITS SUBSTRATES*. J. Biol. Chem. 288, 2092-2102 (2013).
62. Gupta, S. P., Shaik, B. & Prabhakar, Y. S. Advances in Studies on Adenovirus Proteases and Their Inhibitors. in Viral Proteases and Their Inhibitors (ed. Gupta, S. P.) 59-75 (Academic Press, 2017). doi:10.1016/B978-0-12-809712-0.00003-4.
63. Takahashi, E., Cohen, S. L., Tsai, P. K. & Sweeney, J. A. Quantitation of adenovirus type 5 empty capsids. Anal. Biochem. 349, 208-217 (2006).
64. Imelli, N., Ruzsics, Z., Puntener, D., Gastaldelli, M. & Greber, U. F. Genetic reconstitution of the human Adenovirus type 2 temperature-sensitive 1 mutant defective in endosomal escape. Virol. J. 6, 174 (2009).
65. Condezo, G. N. et al. Structures of Adenovirus Incomplete Particles Clarify Capsid Architecture and Show Maturation Changes of Packaging Protein L1 52/55k. J. Virol. 89, 9653-9664 (2015).
66. McKelvey, T., Tang, A., Bett, A. J., Casimiro, D. R. & Chastain, M. T-cell response to adenovirus hexon and DNA-binding protein in mice. Gene Ther. 11, 791-796 (2004).
67. Zheng, R. et al. Changes of Host Immunity Mediated by IFN-γ+ CD8+ T Cells in Children with Adenovirus Pneumonia in Different Severity of Illness. Viruses 13, 2384 (2021).
68. Binder, A. M. et al. Human Adenovirus Surveillance—United States, 2003-2016. MMWR Morb. Mortal. Wkly. Rep. 66, (2017).
69. Vela Ramirez, J. E., Sharpe, L. A. & Peppas, N. A. Current state and challenges in developing oral vaccines. Adv. Drug Deliv. Rev. 114, 116-131 (2017).
70. Wen, K., Resch, M. D., Mazboudi, R., Mulhall Maasz, H. & Galarza, J. M. Novel and efficient method for the reconstruction of adenoviruses through isothermal assembly and its potential applications. Front. Med. Technol. 5, (2023).

1. An adenovirus virus-like particle (AdVLP) comprising:
a recombinant capsid comprising:
a) major capsid adenovirus (AdV) proteins including a hexon protein, a penton protein, and a fiber protein,
b) minor capsid/cement AdV proteins, including a IIIa protein, a VI protein, a VIII protein, and a IX protein, wherein the minor capsid/cement AdV proteins structurally support the major capsid AdV proteins,
c) a chaperone AdV protein L4-100k, and
d) an accessory scaffold AdV protein L1-52/55k.

2. The AdVLP of claim 1, wherein the AdVLP is an adenovirus type 4 virus-like particle (AdVLP4), adenovirus type 7 virus-like particle (AdVLP7), adenovirus type 14 virus-like particle (AdVLP14), or adenovirus type 55 virus-like particle (AdVLP55).

3. The AdVLP of claim 1, wherein the major capsid AdV hexon protein comprises SEQ ID NO: 1 or SEQ ID NO: 2, the major capsid AdV penton protein comprises SEQ ID NO: 3 or SEQ ID NO: 4, and the major capsid AdV fiber protein comprises SEQ ID NO: 5 or SEQ ID NO: 6.

4. The AdVLP of claim 1, wherein the minor capsid/cement Adv protein IIIA protein comprises SEQ ID NO: 11 or SEQ ID NO: 12, the minor capsid/cement Adv protein VI protein comprises protein comprises SEQ ID NO: 13 or SEQ ID NO: 14, the minor capsid/cement Adv protein VIII protein comprises protein comprises SEQ ID NO: 15 or SEQ ID NO: 16, and the minor capsid/cement Adv protein IX protein comprises protein comprises SEQ ID NO: 17 or SEQ ID NO: 18.

5. The AdVLP of claim 1, wherein the chaperone AdV protein L4-100k comprises SEQ ID NO: 7 or SEQ ID NO: 8.

6. The AdVLP of claim 1, wherein the accessory scaffold AdV L1-52/55k protein comprises SEQ ID NO: 9 or SEQ ID NO: 10.

7. (canceled)

8. (canceled)

9. An expression plasmid comprising genes encoding adenovirus proteins, wherein the expression plasmid is suitable for the assembly of adenovirus virus-like particles (AdVLPs) and wherein the expression plasmid comprises codon-optimized genes encoding:
a) major capsid adenovirus (AdV) proteins including a hexon protein, a penton protein, and a fiber protein,
b) minor capsid/cement AdV proteins, including a IIIa protein, a VI protein, a VIII protein, and a IX protein,
c) a chaperone AdV protein L4-100k, and
d) an accessory scaffold AdV protein L1-52/55k.

10. The expression plasmid of claim 9, wherein the AdVLP is an adenovirus type 4 virus-like particle (AdVLP4), adenovirus type 7 virus-like particle (AdVLP7), adenovirus type 14 virus-like particle (AdVLP14), or adenovirus type 55 virus-like particle (AdVLP55).

11. The expression plasmid of claim 9, wherein:
a) the major capsid AdV hexon protein comprises SEQ ID NO: 1 or SEQ ID NO: 2, the major capsid AdV penton protein comprises SEQ ID NO: 3 or SEQ ID NO: 4, and the major capsid AdV fiber protein comprises SEQ ID NO: 5 or SEQ ID NO: 6;
b) the minor capsid/cement Adv protein IIIA protein comprises SEQ ID NO: 11 or SEQ ID NO: 12, the minor capsid/cement Adv protein VI protein comprises protein comprises SEQ ID NO: 13 or SEQ ID NO: 14, the minor capsid/cement Adv protein VIII protein comprises protein comprises SEQ ID NO: 15 or SEQ ID NO: 16, and the minor capsid/cement Adv protein IX protein comprises protein comprises SEQ ID NO: 17 or SEQ ID NO: 18;
c) the chaperone AdV protein L4-100k comprises SEQ ID NO: 7 or SEQ ID NO: 8; and
d) the accessory scaffold AdV protein L1-52/55k comprises SEQ ID NO: 9 or SEQ ID NO: 10.

12. An immunogenic composition comprising at least one AdVLP of claim 1.

13. The immunogenic composition of claim 12, wherein the at least one AdVLP is adenovirus type 4 virus-like particle (AdVLP4), adenovirus type 7 virus-like particle (AdVLP7), adenovirus type 14 virus-like particle (AdVLP14), or adenovirus type 55 virus-like particle (AdVLP55).

14. The immunogenic composition of claim 12, wherein the recombinant capsid of the at least one AdVLP comprises:
a) the major capsid AdV hexon protein comprises SEQ ID NO: 1 or SEQ ID NO: 2, the major capsid AdV penton protein comprises SEQ ID NO: 3 or SEQ ID NO: 4, and the major capsid AdV fiber protein comprises SEQ ID NO: 5 or SEQ ID NO: 6;
b) the minor capsid/cement Adv protein IIIA protein comprises SEQ ID NO: 11 or SEQ ID NO: 12, the minor capsid/cement Adv protein VI protein comprises protein comprises SEQ ID NO: 13 or SEQ ID NO: 14, the minor capsid/cement Adv protein VIII protein comprises protein comprises SEQ ID NO: 15 or SEQ ID NO: 16, and the minor capsid/cement Adv protein IX protein comprises protein comprises SEQ ID NO: 17 or SEQ ID NO: 18;
c) the chaperone AdV protein L4-100k comprises SEQ ID NO: 7 or SEQ ID NO: 8; and
d) the accessory scaffold AdV protein L1-52/55k comprises SEQ ID NO: 9 or SEQ ID NO: 10.

15. The immunogenic composition of claim 13, wherein the composition is a trivalent composition comprising AdVLP4, AdVLP7, and AdVLP14.

16. The immunogenic composition of claim 13, wherein the composition is a quadrivalent composition comprising AdVLP4, AdVLP7, AdVLP14, and AdVLP55.

17. (canceled)

18. A method of generating an immune response to one or more adenoviruses in a subject, the method comprising administering an effective amount of the immunogenic composition of claim 12 to the subject.

19. The method of claim 18, wherein the one or more adenoviruses are selected from the group consisting of HAdV-E type 4, HAdV-B type 7, HAdV-B type 14, and HAdV-B type 55.

20. The method of claim 18, wherein the immune response vaccinates the subject against the one or more adenoviruses.

21. (canceled)

22. A method of producing an adenovirus virus-like particle (AdVLP) comprising:
introducing into a host cell at least one expression plasmid under conditions such that the host cell produces the AdVLP, wherein the at least one expression plasmid comprises codon-optimized genes encoding:
a) major capsid adenovirus (AdV) proteins including a hexon protein, a penton protein, and a fiber protein,
b) minor capsid/cement AdV proteins, including a IIIa protein, a VI protein, a VIII protein, and a IX protein,
c) a chaperone AdV protein L4-100k, and
d) an accessory scaffold AdV protein L1-52/55k.

23. The method of claim 22, wherein:
a) the major capsid AdV hexon protein comprises SEQ ID NO: 1 or SEQ ID NO: 2, the major capsid AdV penton protein comprises SEQ ID NO: 3 or SEQ ID NO: 4, and the major capsid AdV fiber protein comprises SEQ ID NO: 5 or SEQ ID NO: 6;
b) the minor capsid/cement Adv protein IIIA protein comprises SEQ ID NO: 11 or SEQ ID NO: 12, the minor capsid/cement Adv protein VI protein comprises protein comprises SEQ ID NO: 13 or SEQ ID NO: 14, the minor capsid/cement Adv protein VIII protein comprises protein comprises SEQ ID NO: 15 or SEQ ID NO: 16, and the minor capsid/cement Adv protein IX protein comprises protein comprises SEQ ID NO: 17 or SEQ ID NO: 18;
c) the chaperone AdV protein L4-100k comprises SEQ ID NO: 7 or SEQ ID NO: 8; and
d) the accessory scaffold AdV protein L1-52/55k comprises SEQ ID NO: 9 or SEQ ID NO: 10.

24. (canceled)

25. (canceled)

26. (canceled)

27. (canceled)

28. The AdVLP of claim 1, wherein the AdVLP comprises hexon proteins from two different serotypes such that the AdVLP is chimeric.

29. (canceled)

30. (canceled)
